SEQUENCE LISTING (61 sequences)

SEQ ID NO: 1 - protein, 11 aa, Artificial Sequence
  Amino acid sequence corresponding to residues 108 to 118 of LSD1
RRTSRRKRAKV

SEQ ID NO: 2 - protein, 11 aa, Artificial Sequence
  Amino acid sequence corresponding to residues 108 to 118 of LSD1
RRTARRKRAKV

SEQ ID NO: 3 - protein, 13 aa, Artificial Sequence
  Amino acid sequence corresponding to residues 108 to 118 of LSD1
RWRRTARRKRAKV

SEQ ID NO: 4 - protein, 14 aa, Artificial Sequence
  Amino acid sequence consisting of residues 106 to 119 of LSD1
EGRRTSRRKRAKVE

SEQ ID NO: 5 - protein, 852 aa, Artificial Sequence
  Amino acid sequence of LSD1
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 6 - protein, 11 aa, Artificial Sequence
  Cell penetrating peptide
YGRKKRPQRRR

SEQ ID NO: 7 - protein, 16 aa, Artificial Sequence
  Cell penetrating peptide
RRWRRWWRRWWRRWRR

SEQ ID NO: 8 - protein, 20 aa, Artificial Sequence
  Cell penetrating peptide
CWKKKKKKKKKKKKKKKKKK

SEQ ID NO: 9 - protein, 40 aa, Artificial Sequence
  Cell penetrating peptide
KKKKKKKKKKKKKKKKKKWCCWKKKKKKKKKKKKKKKKKK

SEQ ID NO: 10 - protein, 26 aa, Artificial Sequence
  Cell penetrating peptide
WTLNSAGYLLGKINLKALAALAKKIL

SEQ ID NO: 11 - protein, 13 aa, Artificial Sequence
  Cell penetrating peptide
GLFEALEELWEAK

SEQ ID NO: 12 - protein, 46 aa, Artificial Sequence
  Cell penetrating peptide
KKKKKKKKKKKKKKKKGGCRGDMFGCAKKKKKKKKKKKKKKKKRGD

SEQ ID NO: 13 - protein, 25 aa, Artificial Sequence
  Cell penetrating peptide
KKKKKKKKKKKKKKKKGGCMFGCGG

SEQ ID NO: 14 - protein, 31 aa, Artificial Sequence
  Cell penetrating peptide
KKKKKKKKKKKKKKKKICRRARGDNPDDRCT

SEQ ID NO: 15 - protein, 22 aa, Artificial Sequence
  Cell penetrating peptide
  MISC_FEATURE (17)..(17): Xaa is Asn or Asp
  MISC_FEATURE (17)..(17): Xaa is Asn or Asp
  MISC_FEATURE (20)..(20): Xaa is Asn or Asp
  MISC_FEATURE (21)..(21): Xaa is Asn or Asp
KKWKMRRNQFWVKVQRXAKXXA

SEQ ID NO: 16 - protein, 30 aa, Artificial Sequence
  Cell penetrating peptide
VAYISRGGVSTYYSDTVKGRFTRQKYNKRA

SEQ ID NO: 17 - protein, 32 aa, Artificial Sequence
  Cell penetrating peptide
IGRIDPANGKTKYAPKFQDKATRSNYYGNSPS

SEQ ID NO: 18 - protein, 21 aa, Artificial Sequence
  Cell penetrating peptide
KETWWETWWTEWSQPKKKRKV

SEQ ID NO: 19 - protein, 12 aa, Artificial Sequence
  Cell penetrating peptide
PLAEIDGIELTY

SEQ ID NO: 20 - protein, 30 aa, Artificial Sequence
  Cell penetrating peptide
KKKKKKKKKKKKKKKKGGPLAEIDGIELGA

SEQ ID NO: 21 - protein, 30 aa, Artificial Sequence
  Cell penetrating peptide
KKKKKKKKKKKKKKKKGGPLAEIDGIELCA

SEQ ID NO: 22 - protein, 28 aa, Artificial Sequence
  Cell penetrating peptide
GALFLGFLGGAAGSTMGAWSQPKSKRKV

SEQ ID NO: 23 - protein, 28 aa, Artificial Sequence
  Cell penetrating peptide
WEAKLAKALAKALAKHLAKALAKALKAC

SEQ ID NO: 24 - protein, 30 aa, Artificial Sequence
  Cell penetrating peptide
LARLLARLLARLLARLLARLLARLNHCHHH

SEQ ID NO: 25 - protein, 16 aa, Artificial Sequence
  Cell penetrating peptide
KLLKLLLKLWLLKLLL

SEQ ID NO: 26 - protein, 11 aa, Artificial Sequence
  Cell penetrating peptide
KKKKKKKKGGC

SEQ ID NO: 27 - protein, 11 aa, Artificial Sequence
  Cell penetrating peptide
KWKKKWKKGCC

SEQ ID NO: 28 - protein, 11 aa, Artificial Sequence
  Cell penetrating peptide
RWRRRWRRGGC

SEQ ID NO: 29 - protein, 7 aa, Artificial Sequence
  Cell penetrating peptide
PKKKRKV

SEQ ID NO: 30 - protein, 12 aa, Artificial Sequence
  Cell penetrating peptide
PEVKKKRKPEYP

SEQ ID NO: 31 - protein, 12 aa, Artificial Sequence
  Cell penetrating peptide
TPPKKKRKVEDP

SEQ ID NO: 32 - protein, 13 aa, Artificial Sequence
  Cell penetrating peptide
GGGGPKKKRKVGG

SEQ ID NO: 33 - protein, 13 aa, Artificial Sequence
  Cell penetrating peptide
GGGFSTSLRARKA

SEQ ID NO: 34 - protein, 17 aa, Artificial Sequence
  Cell penetrating peptide
CKKKKKKSEDEYPYVPN

SEQ ID NO: 35 - protein, 28 aa, Artificial Sequence
  Cell penetrating peptide
CKKKKKKKSEDEYPYVPNFSTSLRARKA

SEQ ID NO: 36 - protein, 25 aa, Artificial Sequence
  Cell penetrating peptide
LVRKKRKTEEESPLKDKDAKKSKQE

SEQ ID NO: 37 - protein, 30 aa, Artificial Sequence
  Cell penetrating peptide
KKKKKKKKKKKKKKKKKKKKKKKGGKKKKK

SEQ ID NO: 38 - protein, 13 aa, Artificial Sequence
  Cell penetrating peptide
GRKKRRQRRRPPQ

SEQ ID NO: 39 - protein, 16 aa, Artificial Sequence
  Cell penetrating peptide
RQIKIWFQNRRMKWKK

SEQ ID NO: 40 - protein, 21 aa, Artificial Sequence
  Cell penetrating peptide
TRSSRAGLQFPVGRVHRLLRK

SEQ ID NO: 41 - protein, 13 aa, Artificial Sequence
  Cell penetrating peptide
KRVSRNKSEKKRR

SEQ ID NO: 42 - protein, 18 aa, Artificial Sequence
  Cell penetrating peptide
KLALKLALKALKAALKLA

SEQ ID NO: 43 - protein, 16 aa, Artificial Sequence
  Cell penetrating peptide
AAVALLPAVLLALLAP

SEQ ID NO: 44 - protein, 6 aa, Artificial Sequence
  Cell penetrating peptide
VPMLKE

SEQ ID NO: 45 - protein, 5 aa, Artificial Sequence
  Cell penetrating peptide
VPMLK

SEQ ID NO: 46 - protein, 5 aa, Artificial Sequence
  Cell penetrating peptide
PMLKE

SEQ ID NO: 47 - protein, 4 aa, Artificial Sequence
  Cell penetrating peptide
PMLK

SEQ ID NO: 48 - protein, 28 aa, Artificial Sequence
  Cell penetrating peptide
MANLGYWLLALFVTMWTDVGLCKKRPKP

SEQ ID NO: 49 - protein, 18 aa, Artificial Sequence
  Cell penetrating peptide
LLIILRRRIRKQAHAHSK

SEQ ID NO: 50 - protein, 21 aa, Artificial Sequence
  Cell penetrating peptide
KETWWETWWTEWSQPKKKRKV

SEQ ID NO: 51 - protein, 18 aa, Artificial Sequence
  Cell penetrating peptide
RGGRLSYSRRRFSTSTGR

SEQ ID NO: 52 - protein, 27 aa, Artificial Sequence
  Cell penetrating peptide
GWTLNSAGYLLGKINLKALAALAKKIL

SEQ ID NO: 53 - protein, 21 aa, Artificial Sequence
  Cell penetrating peptide
AGYLLGKINLKALAALAKKIL

SEQ ID NO: 54 - protein, 21 aa, Artificial Sequence
  Cell penetrating peptide
  MISC_FEATURE (21)..(21): Xaa is cysteamide
GLWRALWRLLRSLWRLLWRAX

SEQ ID NO: 55 - protein, 15 aa, Artificial Sequence
  Cell penetrating peptide
SDLWEMMMVSLACQY

SEQ ID NO: 56 - protein, 12 aa, Artificial Sequence
  Cell penetrating peptide
TSPLNIHNGQKL

SEQ ID NO: 57 - protein, 26 aa, Artificial Sequence
  Cell penetrating peptide
DPKGDPKGVTVTVTVTVTGKGDPKPD

SEQ ID NO: 58 - protein, 16 aa, Artificial Sequence
  Cell penetrating peptide
RVIRVWFQNKRCKDKK

SEQ ID NO: 59 - protein, 12 aa, Artificial Sequence
  L1
  MISC_FEATURE (1)..(1): Xaa is myristoyl
  MISC_FEATURE (12)..(12): Xaa is Val-OH
XRRTSRRKRAKX

SEQ ID NO: 60 - protein, 12 aa, Artificial Sequence
  L2
  MISC_FEATURE (1)..(1): Xaa is myristoyl
  MISC_FEATURE (12)..(12): Xaa is Val-OH
XRRTARRKRAKX

SEQ ID NO: 61 - protein, 14 aa, Artificial Sequence
  L3
  MISC_FEATURE (1)..(1): Xaa is myristoyl
  MISC_FEATURE (14)..(14): Xaa is Val-OH
XRWRRTARRKRAKX

(19) United States
(12) Patent Application Publication
(10) Pub. No.: US 2022/0040280 A1
(43) Pub. Date: Feb. 10, 2022

(54) INHIBITORS AND USES THEREOF

(71) Applicant: UNIVERSITY OF CANBERRA, Bruce, Australian Capital Territory (AU)

(72) Inventors: Sudha RAO, Deakin (AU); Peter MILBURN, O'Connor (AU)

(21) Appl. No.: 16/330,628

(22) PCT Filed: Sep. 7, 2017
(86) PCT No.: PCT/AU2017/050969

§ 371 Date: Aug. 12, 2021

(30) Foreign Application Priority Data
Sep. 7, 2016 (AU) 2016903602

(51) Int. Cl. A61K 39/00; A61K 39/39; A61P 35/00; C12N 9/02

(52) U.S. Cl. CPC A61K 39/001154; A61K 39/39; C12Y 114/99; C12N 9/0083; A61P 35/00

(57) ABSTRACT

Disclosed are lysine specific histone demethylase-1 (LSD1) inhibitors in methods and compositions for immune check-point inhibition. The invention also relates to proteinaceous molecules and their use in altering at least one of (i) formation, (ii) 5 N proliferation, (iii) maintenance, (iv) epithelial to mesenchymal cell transition (EMT), or (v) mesenchymal to epithelial cell transition (MET) of an LSD1 overexpressing cell.

This application claims priority to Australian Provisional Application No. 2016903602 entitled “Inhibitors and Uses Therefor” filed on 7 Sep. 2016, the entire content of which is hereby incorporated herein by reference.

### FIELD OF THE INVENTION

This invention relates generally to lysine specific histone demethylase-1 (LSD1) inhibitors in methods and compositions for immune checkpoint inhibition. The invention also relates to proteinaceous molecules and their use in altering at least one of (i) formation, (ii) proliferation, (iii) maintenance, (iv) epithelial to mesenchymal cell transition (EMT), or (v) mesenchymal to epithelial cell transition (MET) of an LSD1 overexpressing cell.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Programmed cell death protein-1 (PD-1) plays an important role in regulation of the immune system through its ability to regulate T cell activation and reduce the immune response. PD-1 is expressed on activated T cells (including immunosuppressive CD4+ T cells (Treg) and exhausted CD8+ T cells), B cells, myeloid dendritic cells (MDCs), monocytes, thymocytes and natural killer (NK) cells (Gianchecchi et al. (2013) Autoimmun. Rev., 12: 1091-1100).

The PD-1 signaling pathway contributes to the maintenance of central and peripheral tolerance in normal individuals, thereby avoiding destruction of normal host tissue. In the thymus, the interaction of PD-1 and its ligands suppresses positive selection, thereby inhibiting the transformation of CD4− CD8− double negative cells to CD4+ CD8+ double positive T cells (Keir et al. (2005) J. Immunol., 175: 7329-7379). Inhibition of self-reactive and inflammatory effector T cells that escape negative selection to avoid collateral immune-mediated tissue damage is dependent on the PD-1 signaling pathway (Keir et al. (2006) J. Exp. Med., 203: 883-895).

PD-1 is bound by two ligands: programmed cell death ligand-1 (PD-L1; B7-H1; CD274) and programmed cell death ligand-2 (PD-L2; B7-DC; CD273). PD-L1 is expressed on various cell types, including T cells, B cells, dendritic cells, macrophages, epithelial cells and endothelial cells (Chen et al. (2012) Clin Cancer Res, 18(24): 6580-6587; Herzberg et al. (2016) The Oncologist, 21: 1-8). PD-L1 expression is also upregulated in many types of tumor cells and other cells in the local tumor environment (Herzberg et al. (2016) The Oncologist, 21: 1-8). PD-L2 is predominantly expressed on antigen-presenting cells such as monocytes, macrophages and dendritic cells, but expression may also be induced on a wide variety of other immune cells and non-immune cells depending on microenvironmental stimuli (Herzberg et al. (2016) The Oncologist, 21: 1-8; Kinter et al. (2008) J. Immunol., 181: 6738-6746; Zhong et al. (2007) Eur. J. Immunol., 37: 2405-2410; Messal et al. (2011) Mol. Immunol., 48: 2214-2219; Lesterhuis et al. (2011) Mol. Immunol., 49: 1-3).

PD-1, PD-L1 and PD-L2 are overexpressed by malignant cells and other cells in the local tumor environment. PD-1 is highly expressed on a large proportion of tumor-infiltrating lymphocytes (TILs) from many different tumor types and suppresses local effector immune responses. TIL expression of PD-1 is associated with impaired effector function (cytokine production and cytotoxic efficacy against tumor cells) and/or poor outcome in numerous tumor types (Thompson et al. (2007) Clin Cancer Res, 13(6): 1757-1761; Shi et al. (2011) Int. J. Cancer, 128: 887-896). PD-L1 expression has been found to strongly correlate with poor outcome in many tumor types, including kidney, ovarian, bladder, breast, urothelial, gastric and pancreatic cancer (Keir et al. (2008) Annu. Rev. Immunol., 26: 677-704; Shi et al. (2011) Int. J. Cancer, 128: 887-896). PD-L2 has been shown to be upregulated in a subset of tumors and has also been linked to poor outcome.

Accordingly, members of the PD-1 signaling pathway are important therapeutic targets for the treatment of cancer and infection and new therapeutic agents targeting this pathway are desired.

### SUMMARY OF THE INVENTION

The present invention is predicated in part on the discovery that LSD1 inhibitors inhibit immune checkpoints, particularly PD-L1 and/or PD-L2. Accordingly, the inventors have conceived that LSD1 inhibitors may be used for a range of applications, including for enhancing an immune response in a subject to a target antigen by an immune-modulating agent or for the treatment of a cancer, particularly a metastatic cancer, or an infection.

Accordingly, in one aspect of the invention, there is provided a use of a LSD1 inhibitor for inhibiting PD-L1 and/or PD-L2 activity in a subject.

In another aspect of the invention, there is provided a method of inhibiting PD-L1 and/or PD-L2 activity in a subject, comprising administering an LSD1 inhibitor to the subject. In a related aspect of the invention, there is provided a method of inhibiting the nuclear translocation of PD-L1 and/or PD-L2 in subject, comprising administering an LSD1 inhibitor to the subject.

In still another aspect of the invention, there is provided a use of an LSD1 inhibitor for enhancing an immune response in a subject to a target antigen by an immune-modulating agent. In a related aspect, the present invention provides a composition comprising an LSD1 inhibitor and an immune-modulating agent.

In a further aspect, the present invention provides a use of an LSD1 inhibitor for enhancing the efficacy of an anti-infective agent. In a related aspect of the present invention, a composition is provided comprising an LSD1 inhibitor and an anti-infective agent.

In yet another aspect of the invention, there is provided a method of enhancing an immune response in a subject to a target antigen by an immune-modulating agent, comprising administering an LSD1 inhibitor to the subject.

In a further aspect of the invention, there is provided a method of inhibiting PD-L1 and/or PD-L2 activity comprising contacting a PD-L1 and/or PD-L2 overexpressing cell with an LSD1 inhibitor.

In a still further aspect of the invention, there is provided a use of an LSD1 inhibitor for inhibiting PD-L1 and/or PD-L2 activity comprising contacting a PD-L1 and/or PD-L2 overexpressing cell with an LSD1 inhibitor. In a related aspect of the invention, there is provided a method of inhibiting the nuclear translocation of PD-L1 and/or PD-L2 in a PD-L1 and/or PD-L2 overexpressing cell, comprising contacting the PD-L1 and/or PD-L2 overexpressing cell with an LSD1 inhibitor.

The present inventors have also conceived that proteinaceous molecules based on a subsequence of the LSD1 polypeptide and structurally-related molecules inhibit LSD1 activity, including the translocation of LSD1 into the nucleus of a cell, and are also useful therefore for inhibiting PD-L1 and/or PD-L2 activity, as broadly described above and elsewhere herein. Furthermore, such proteinaceous molecules are also proposed to inhibit the phosphorylating activity of a PKC, especially PKC-θ. These molecules have also been shown to have significant activity in inhibiting EMT, in inhibiting formation and maintenance of cancer stem cell and non-cancer stem cell tumor cells, and in inducing MET, which makes them useful in treating a range of conditions, associated with LSD1 overexpression, such as cancer.

Thus, another aspect of the invention provides a method of inhibiting the phosphorylating activity of a protein kinase C (PKC), comprising contacting a PKC overexpressing cell with an isolated or purified proteinaceous molecule comprising or consisting essentially of a sequence corresponding to residues 108 to 118 of LSD1.

In yet another aspect of the invention, there is provided a method of altering at least one of (i) formation; (ii) proliferation; (iii) maintenance; (iv) EMT; or (v) MET of a PKC overexpressing cell, comprising contacting said PKC overexpressing cell with a formation-, proliferation-, maintenance-, EMT- or MET-modulating amount of an isolated or purified proteinaceous molecule comprising, consisting or consisting essentially of a sequence corresponding to residues 108 to 118 of LSD1.

The present invention also provides a method of treating or preventing a cancer in a subject, wherein the cancer comprises at least one PKC overexpressing cell, comprising administering to the subject an isolated or purified proteinaceous molecule comprising, consisting or consisting essentially of a sequence corresponding to residues 108 to 118 of LSD1.

In another aspect of the invention, there is provided a method of inhibiting PKC phosphorylation of LSD1 in an LSD1 overexpressing cell, comprising contacting the LSD1 overexpressing cell with an isolated or purified proteinaceous molecule comprising, consisting or consisting essentially of a sequence corresponding to residues 108 to 118 of LSD1.

In yet another aspect of the invention, there is provided a method of inhibiting an activity of LSD1, comprising contacting an LSD1 overexpressing cell with an isolated or purified proteinaceous molecule comprising, consisting or consisting essentially of a sequence corresponding to residues 108 to 118 of LSD1.

A further aspect of the invention provides a method of inhibiting the nuclear translocation of LSD1 in an LSD1 overexpressing cell, comprising contacting the LSD1 overexpressing cell with an isolated or purified proteinaceous molecule comprising, consisting or consisting essentially of a sequence corresponding to residues 108 to 118 of LSD1.

In a still further aspect of the invention, there is provided a method of altering at least one of (i) formation; (ii) proliferation; (iii) maintenance; (iv) EMT; or (v) MET of an LSD1 overexpressing cell, comprising contacting said LSD1 overexpressing cell with a formation-, proliferation-, maintenance-, EMT- or MET-modulating amount of an isolated or purified proteinaceous molecule comprising, consisting or consisting essentially of a sequence corresponding to residues 108 to 118 of LSD1.

The invention also extends to a method of treating or preventing a cancer in a subject, wherein the cancer comprises at least one LSD1 overexpressing cell, comprising administering to the subject an isolated or purified proteinaceous molecule comprising, consisting or consisting essentially of a sequence corresponding to residues 108 to 118 of LSD1.

In a still further aspect of the invention, there is provided a use of an isolated or purified proteinaceous molecule comprising, consisting or consisting essentially of a sequence corresponding to residues 108 to 118 of LSD1 for inhibiting the phosphorylating activity of a PKC in a PKC overexpressing cell.

The present invention, in another aspect, provides a use of an isolated or purified proteinaceous molecule comprising, consisting or consisting essentially of a sequence corresponding to residues 108 to 118 of LSD1 in the manufacture of a medicament for inhibiting the phosphorylating activity of a PKC in a PKC overexpressing cell.

In another aspect of the invention, there is provided a use of an isolated or purified proteinaceous molecule comprising, consisting or consisting essentially of a sequence corresponding to residues 108 to 118 of LSD1 for altering at least one of (i) formation; (ii) proliferation; (iii) maintenance; (iv) EMT; or (v) MET of a PKC overexpressing cell.

In a further aspect of the invention, there is provided a use of an isolated or purified proteinaceous molecule comprising, consisting or consisting essentially of a sequence corresponding to residues 108 to 118 of LSD1 in the manufacture of a medicament for altering at least one of (i) formation; (ii) proliferation; (iii) maintenance; (iv) EMT; or (v) MET of a PKC overexpressing cell.

The present invention also contemplates a use of an isolated or purified proteinaceous molecule comprising, consisting or consisting essentially of a sequence corresponding to residues 108 to 118 of LSD1 for treating or preventing a cancer in a subject, wherein the cancer comprises at least one PKC overexpressing cell.

In another aspect, the present invention provides a use of an isolated or purified proteinaceous molecule comprising a sequence corresponding to residues 108 to 118 of LSD1 in the manufacture of a medicament for treating or preventing a cancer in a subject, wherein the cancer comprises at least one PKC overexpressing cell.

In still another aspect of the invention, there is provided a use of an isolated or purified proteinaceous molecule comprising, consisting or consisting essentially of a sequence corresponding to residues 108 to 118 of LSD1 for inhibiting PKC phosphorylation of LSD1 in an LSD1 overexpressing cell.

In yet another aspect of the invention, there is provided a use of an isolated or purified proteinaceous molecule comprising, consisting or consisting essentially of a sequence corresponding to residues 108 to 118 of LSD1 in the manufacture of a medicament for inhibiting PKC phosphorylation of LSD1 in an LSD1 overexpressing cell.

In a further aspect, the present invention provides a use of an isolated or purified proteinaceous molecule comprising, consisting or consisting essentially of a sequence corresponding to residues 108 to 118 of LSD1 for inhibiting an activity of LSD1.

The present invention contemplates a use of an isolated or purified proteinaceous molecule comprising, consisting or consisting essentially of a sequence corresponding to residues 108 to 118 of LSD1 in the manufacture of a medicament for inhibiting an activity of LSD1.

In another aspect of the invention, there is provided a use of an isolated or purified proteinaceous molecule comprising, consisting or consisting essentially of a sequence corresponding to residues 108 to 118 of LSD1 for inhibiting the nuclear translocation of LSD1 in an LSD1 overexpressing cell.

In still another aspect of the invention, there is provided a use of an isolated or purified proteinaceous molecule comprising, consisting or consisting essentially of a sequence corresponding to residues 108 to 118 of LSD1 in the manufacture of a medicament for inhibiting the nuclear translocation of LSD1 in an LSD1 overexpressing cell.

The present invention also extends to a use of an isolated or purified proteinaceous molecule comprising, consisting or consisting essentially of a sequence corresponding to residues 108 to 118 of LSD1 for altering at least one of (i) formation; (ii) proliferation; (iii) maintenance; (iv) EMT; or (v) MET of an LSD1 overexpressing cell.

In a further aspect of the invention, there is provided a use of an isolated or purified proteinaceous molecule comprising, consisting or consisting essentially of a sequence corresponding to residues 108 to 118 of LSD1 in the manufacture of a medicament for altering at least one of (i) formation; (ii) proliferation; (iii) maintenance; (iv) EMT; or (v) MET of an LSD1 overexpressing cell.

In a still further aspect of the invention, there is provided a use of an isolated or purified proteinaceous molecule comprising, consisting or consisting essentially of a sequence corresponding to residues 108 to 118 of LSD1 for treating or preventing a cancer in a subject, wherein the cancer comprises at least one LSD1 overexpressing cell.

Yet a further aspect of the invention provides a use of an isolated or purified proteinaceous molecule comprising, consisting or consisting essentially of a sequence corresponding to residues 108 to 118 of LSD1 in the manufacture of a medicament for treating or preventing a cancer in a subject, wherein the cancer comprises at least one LSD1 overexpressing cell.

Another aspect of the invention provides an isolated or purified proteinaceous molecule comprising, consisting or consisting essentially of a sequence corresponding to residues 108 to 118 of LSD1 for use in inhibiting the phosphorylating activity of a PKC in a PKC overexpressing cell.

In yet another aspect of the invention, there is provided an isolated or purified proteinaceous molecule comprising, consisting or consisting essentially of a sequence corresponding to residues 108 to 118 of LSD1 for use in altering at least one of (i) formation; (ii) proliferation; (iii) maintenance; (iv) EMT; or (v) MET of a PKC overexpressing cell.

In still another aspect of the invention, there is provided an isolated or purified proteinaceous molecule comprising, consisting or consisting essentially of a sequence corresponding to residues 108 to 118 of LSD1 for use in treating or preventing a cancer in a subject, wherein the cancer comprises at least one PKC overexpressing cell.

In a further aspect, the invention provides an isolated or purified proteinaceous molecule comprising, consisting or consisting essentially of a sequence corresponding to residues 108 to 118 of LSD1 for use in inhibiting PKC phosphorylation of LSD1 in an LSD1 overexpressing cell.

In yet a further aspect of the invention, there is provided an isolated or purified proteinaceous molecule comprising, consisting or consisting essentially of a sequence corresponding to residues 108 to 118 of LSD1 for use in inhibiting an activity of LSD1.

The invention also contemplates an isolated or purified proteinaceous molecule comprising, consisting or consisting essentially of a sequence corresponding to residues 108 to 118 of LSD1 for use in inhibiting the nuclear translocation of LSD1 in an LSD1 overexpressing cell.

In another aspect of the invention, there is provided an isolated or purified proteinaceous molecule comprising, consisting or consisting essentially of a sequence corresponding to residues 108 to 118 of LSD1 for use in altering at least one of (i) formation; (ii) proliferation; (iii) maintenance; (iv) EMT; or (v) MET of an LSD1 overexpressing cell.

In still another aspect, the invention provides an isolated or purified proteinaceous molecule comprising, consisting or consisting essentially of a sequence corresponding to residues 108 to 118 of LSD1 for use in treating or preventing a cancer in a subject, wherein the cancer comprises at least one LSD1 overexpressing cell.

In a further aspect of the invention, there is provided an isolated or purified proteinaceous molecule represented by Formula I:

Z₁RRTX₁RRKRAKVZ₂ (I)

wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof;
wherein the proteinaceous molecule is other than a proteinaceous molecule consisting of the amino acid sequence of SEQ ID NO: 4:

[SEQ ID NO: 4]
EGRRTSRRKRAKVE.

### BRIEF DESCRIPTION OF THE DRAWINGS

FIG. 1 is a photographic and graphical representation of expression of PD-L1 and LSD1 phosphorylated at serine 111 (LSD1s111p) in MCF7 [stimulated with PMA (MCF7ST) and non-stimulated (MCF7NS)] and MDA-MB-231 (MDA) breast cancer cell lines. Total nuclear fluorescence (TNFI), total cytoplasmic fluorescence (TCFI), the nuclear bias (Fn/c) and the degree of co-localization (PCC) is presented.

FIG. 2 is a photographic and graphical representation of expression of LSD1 phosphorylated at serine 111 (LSD1p) and PD-L2 in MDA-MB-231 breast cancer cell lines. Total nuclear fluorescence (TNFI), total cytoplasmic fluorescence (TCFI), the nuclear bias (Fn/c) and the degree of co-localization (PCC) is presented.

FIG. 3 depicts the expression of CD44, PD-L1 and PD-L2 in MCF7 [adherent cells stimulated with PMA (MCF7 ST AD); suspension cells stimulated with PMA (MCF7 ST SUS); and non-stimulated (MCF7 NS)] and MDA-MB-231 cells. FACS plots of inducible MCF7 cells non-stimulated, stimulated with PMA or stimulated with PMA and TGF-β and stained for PD-L1 are presented in FIG. 3A. FIG. 3B depicts the expression of CD44, PD-L1 and PD-L2 mRNA in MCF7 and MDA-MB-231 cells. FIG. 3C depicts the expression of CD44, PD-L1 and PD-L2 mRNA in MCF7 cells treated with LSD1 siRNA.

FIG. 4 is a graphical and photographic representation of the effect of peptide inhibitors L1, L2 and L3 on the expression and nuclear localization of LSD1 phosphorylated at serine 111 (LSD1p) in stimulated (MCF7ST) and non-stimulated (MCF7NS) MCF7 cells and MDA-MB-231 cells (MDA). Total nuclear fluorescence (TNFI) was determined.

FIG. 5 is a graphical and photographic representation of the effect of peptide inhibitors L1, L2 and L3 on the expression and nuclear localization of PD-L1 in stimulated (MCF7ST) and non-stimulated (MCF7NS) MCF7 cells. Total nuclear fluorescence (TNFI), total cytoplasmic fluorescence (TCFI) and the nuclear bias (Fn/c) were determined.

FIG. 6 is a graphical and photographic representation of the effect of peptide inhibitors L1, L2 and L3 on the expression and nuclear localization of PD-L1 in MDA-MB-231 cells. Total nuclear fluorescence (TNFI), total cytoplasmic fluorescence (TCFI) and the nuclear bias (Fn/c) were determined.

FIG. 7 is a graphical and photographic representation of the effect of treatment of a mouse MDA-MB-231 xenograft with vehicle (Con), Abraxane (60 mg/kg, 30 mg/kg and 10 mg/kg) (Abrax) or Docetaxel (10 mg/kg and 4 mg/kg) (Doc). FIG. 7A shows the volume of the tumors over time during treatment. FIG. 7B shows the nuclear fluorescent signal (TNFI) of (i) LSD1 phosphorylated at serine 111 (LSD1s111p), (ii) epidermal growth factor receptor (EGFR), and (iii) SNAIL in Abraxane or Docetaxel treated cells relative to the vehicle alone.

FIG. 8 is a graphical and photographic representation of the effect of treatment with vehicle (Con), Abraxane (60 mg/kg) (Abrax), Phenelzine (41 mg/kg, 27 mg/kg or 13.5 mg/kg) (Phenel) or a combination thereof on mouse xenograft MDA-MB-231 cells. FIGS. 8A-E depict the tumor size and volume over time.

FIG. 9 is a graphical and photographic representation of the effect of treatment with Abraxane (60 mg/kg) (Abrax), Phenelzine (41 mg/kg) (Phenel) or a combination thereof on mouse xenograft MDA-MB-231 cells. FIG. 9 depicts the expression of LSD1 phosphorylated at serine 111 (LSD1s111p), cytokeratin and PD-L1. This figure assesses the total nuclear fluorescence (TNFI), total cytoplasmic fluorescence (TCFI), nuclear bias (Fn/c) and the degree of co-localization (PCC).

FIG. 10 is a graphical and photographic representation of the effect of treatment with Abraxane (60 mg/kg) (Abrax), Phenelzine (41 mg/kg) (Phenel) or a combination thereof on mouse xenograft MDA-MB-231 cells. This figure assess the nuclear (TNFI) and cytoplasmic (TCFI) expression of EGFR and cell surface vimentin (CSV).

FIG. 11 is a graphical and photographic representation of the effect of treatment with vehicle (Mock; Group A), Abraxane (60 mg/kg) (Group B), or Docetaxel (10 mg/kg) (Group E) on mouse xenograft MDA-MB-231 cells. This figure presents the expression of PD-L2 and the mesenchymal marker, MET. This figure assesses the total nuclear fluorescence (TNFI), total cytoplasmic fluorescence (TCFI), nuclear bias (Fn/c) and the degree of co-localization (PCC).

FIG. 12 presents representative images of circulating tumor cells isolated from patient liquid biopsies. The images show the expression of SNAIL, a transcription factor implicated in aggressive cancer with a mesenchymal state; vimentin and cyctokeratin, which are markers for circulating tumor cells; LSD1 phosphorylated at serine 111 (LSD1s111p); PD-L1; and PD-L2.

FIG. 13 is a photographic (FIG. 13A) and graphical (FIG. 13B) representation of the expression of LSD1 phosphorylated at serine 111 (LSD1s111p), PD-L1 and cell surface vimentin (CSV) in circulating tumor cells isolated from breast cancer patient liquid biopsies. Total nuclear fluorescence (TNFI), cytoplasmic fluorescence (TCFI), nuclear bias (Fn/c) and the degree of co-localization (PCC) are depicted.

FIG. 14 is a photographic and graphical representation of the expression of of LSD1 phosphorylated at serine 111 (LSD1s111p), PD-L2 and cytokeratin in circulating tumor cells isolated from breast cancer patient liquid biopsies. Total nuclear fluorescence (TNFI), cytoplasmic fluorescence (TCFI), nuclear bias (Fn/c) and the degree of co-localization (PCC) are depicted.

FIG. 15 is a photographic (FIG. 15A) and graphical (FIG. 15B) representation of the expression of cell surface vimentin, SNAIL and PD-L1 in circulating tumor cells isolated from breast cancer patient liquid biopsies. Total nuclear fluorescence (TNFI), cytoplasmic fluorescence (TCFI), nuclear bias (Fn/c) and the degree of co-localization (PCC) are depicted.

FIG. 16 depicts the total cytoplasmic expression (TCFI) of LSD1 phosphorylated at serine 111 (LSD1s111p), cytokeratin and PD-L2 in circulating tumor cells isolated from breast cancer patient liquid biopsies in response to treatment with Pargyline (Parg) and Phenelzine.

FIG. 17 is a photographic and graphical representation of the expression of cell surface vimentin, PD-L1 and SNAIL in circulating tumor cells isolated from breast cancer patient liquid biopsies in response to treatment with Pargyline (FIG. 17A) and Phenelzine (FIG. 17B) in comparison to vehicle (Mock). The total cell fluorescence (TCFI) is presented.

FIG. 18 presents the effect of cyclin-dependent kinase (CDK) inhibitors Palbociclib (Palbo) or Ribociclib (Ribo), Phenelzine (Phenel) and combinations thereof on the expression of PD-L1 in the nucleus (nPD-L1) and surface (sPD-L1) of circulating tumor cells isolated from two breast cancer patient liquid biopsies, where FIG. 18A presents the data obtained from Patient A and FIG. 18B presents the data obtained from Patient B. FACS data and graphical representations thereof are presented.

FIG. 19 is a graphical and photographic representation of the effect of three peptide LSD1 inhibitors, L1, L2 and L3, on nuclear translocation of LSD1 in MDA-MB-231 cells in comparison to control (Ctrl). The nuclear bias (Fn/c) of LSD1 expression is presented.

FIG. 20 depicts the effect of the LSD1 peptide inhibitors, L1, L2 and L3 (50 μM and 100 μM) on cancer stem cell (CSC) formation in MCF7 cells. FACS data and graphical representations thereof are presented.

FIG. 21 depicts the effect of the LSD1 peptide inhibitors, L1, L2 and L3 (50 μM and 100 μM) on cancer stem cell (CSC) formation in MDA-MB-231 cells. FACS data and graphical representations thereof are presented.

FIG. 22 is a photographic and graphical representation of the effect of the LSD1 peptide inhibitors, L1, L2 and L3 (50 μM and 100 μM) on the expression of PD-L1 in MDA-MB-231 (MDA) cells in comparison to control (Mock). Total cell fluorescence (TFI) is presented.

FIG. 23 is a photographic and graphical representation of the effect of the LSD1 peptide inhibitors, L1, L2 and L3 on the expression of epidermal growth factor receptor (EGFR) in MDA-MB-231 (MDA) cells in comparison to control (Pep-Ctrl). Total nuclear fluorescence (TNFI) is presented.

FIG. 24 is a photographic and graphical representation of the effect of L1, L2 and L3 on the expression of MET (a mesenchymal marker) in MDA-MB-231 (MDA) cells in comparison to control (Pep-Ctrl). Total nuclear fluorescence (TNFI) is presented.

FIG. 25 is a photographic and graphical representation of the expression of PKC-6 and LSD1 in non-stimulated (MCF7NS), PMA stimulated adherent (MCF7 ST) and PMA stimulated floating (MCF7 FLT) MCF7 cells, and MDA-MB-231 (MDA) cells. Data represent the mean±SEM. The plot profile feature of ImageJ was used to plot the fluorescence signal intensity alone a single line spanning the nucleus (n=5 lines per nucleus, 5 individual cells). The average fluorescence signal intensity for the indicated pair of antibodies was plotted for each point on the line with standard error. Signal was plotted to compare how the signals for each antibody varied compared to the opposite antibody. The degree of co-localization (PCC) was determined for each plot profile. Total nuclear fluorescence (TNFI) is also presented.

FIG. 26 is a photographic and graphical representation of the expression of LSD1 phosphorylated at serine 111 (LSD1s111p) in non-stimulated (MCF7NS) and stimulated (MCF7ST) MCF7 cells and MDA-MB-231 (MDA) cells in response to treatment with the PKC-θ inhibitors C27 or BIM. Total nuclear fluorescence (TNFI) is presented. Data shown represented mean±SEM for at least 20 individual cells.

FIG. 27 depicts the effect of Abraxane (60 mg/kg) and Docetaxel (10 mg/kg) treatment on expression of LSD1 phosphorylated at serine 111 (LSD1s111p) in xenografted MDA-MB-231 cells in comparison to control (Mock). Representative images and a graph presenting the total nuclear fluorescence (TNFI) are presented. Data shown represents the mean±SEM for at least 20 individual cells.

FIG. 28 is a photographic and graphical representation of the effect of LSD1 siRNA (si-LSD1) on the expression of LSD1 and SNAIL in PMA stimulated (ST) and non-stimulated (NS) MCF7 cells in comparison to control (Mock). Data presented represents the mean±SEM for at least 20 cells.

FIG. 29 is a photographic and graphical representation of the effect of inhibitors of the catalytic activity of LSD1, NCD36 (NCB) and pargyline (Parg), on the expression of LSD1 and SNAIL in PMA stimulated (ST) and non-stimulated (NS) MCF7 cells in comparison to control (Mock). Data presented represents the mean±SEM for at least 20 cells.

FIG. 30 is a photographic and graphical representation of the effect of LSD1 siRNA (si-LSD1), pargyline (Parg) and NCD36 (NCB) on the expression of LSD1 and SNAIL in MDA-MB-231 cells in comparison to control (Mock). Data presented represents the mean±SEM for at least 20 cells.

FIG. 31 is a photographic and graphical representation of the effect of the peptide inhibitors, L1, L2 and L3, on the expression of LSD1 in MDA-MB-231 cells in comparison to control (Ctrl). Data presented represents the mean±SEM for at least 20 cells. The nuclear bias (Fn/c) of LSD1 expression is presented.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The terms “administration concurrently”, “administering concurrently” or “administered concurrently” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and preferably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example, at an interval of about one day to several weeks or months. The active agents may be administered in any order. The term “sequentially” as used herein means that the agents are administered in sequence, for example, at an interval of minutes, hours, days or weeks. If appropriate, the active agents may be administered in a regular repeating cycle.

The term “agent” or “modulatory agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

As used herein, the term “alkyl” refers to a straight chain, branched or cyclic saturated hydrocarbon group having 1 to 10 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl, decyl, cyclopropyl, cyclobutyl, cyclopentyl, cyclohexyl and cycloheptyl.

As used herein, the term “alkenyl” refers to a straight-chain, branched or cyclic hydrocarbon group having one or more double bonds between carbon atoms and having 2 to 10 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, cyclopentenyl, cyclohexenyl and cyclohexadienyl.

“Aralkyl” means alkyl as defined above which is substituted with an aryl group as defined above, e.g., —CH₂phenyl, —(CH₂)₂phenyl, —(CH₂)₃phenyl, —H₂CH(CH₃)CH₂phenyl, and the like and derivatives thereof.

As used herein, “aromatic” or “aryl” is intended to mean any stable monocyclic or bicyclic carbon ring of up to 7 atoms in each ring, wherein at least one ring is aromatic. Examples of such aryl elements include, but are not limited to, phenyl, naphthyl, tetrahydronaphthyl, indanyl, biphenyl, phenanthryl, anthryl or acenaphthyl.

In certain instances, substituents may be defined with a range of carbons that includes zero, such as (C₀-C₆)alkylene-aryl. If aryl is taken to be phenyl, this definition would include phenyl itself as well as, for example, —CH₂Ph, —CH₂CH₂Ph, CH(CH₃)CH₂CH(CH₃)Ph.

It will also be recognized that the compounds described herein may possess asymmetric centers and are therefore capable of existing in more than one stereoisomeric form. The invention thus also relates to compounds in substantially pure isomeric form at one or more asymmetric centers e.g. greater than about 90% ee, such as about 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

As used herein, the term “and/or” refers to and encompasses any and all possible combinations of one or more of the associated listed items, as well as the lack of combinations when interpreted in the alternative (or).

The term “antigen” as used herein to refer to all, or part of, a protein, peptide, or other molecule or macromolecule capable of eliciting an immune response in a vertebrate animal, especially a mammal. Such antigens are also reactive with antibodies from animals immunized with that protein, peptide, or other molecule or macromolecule.

The term “antigen-binding molecule” is used herein to refer to a molecule that has binding affinity for a target antigen. It will be understood that this term extends to immunoglobulins, immunoglobulin fragments and non-immunoglobulin derived protein frameworks that exhibit antigen-binding activity.

The term “cancer stem cell” (CSC) refers to a cell that has tumor-initiating and tumor-sustaining capacity, including the ability to extensively proliferate, form new tumors and maintain cancer development, i.e. cells with indefinite proliferative potential that drive the formation and growth of tumors. CSCs are biologically distinct from the bulk tumor cells and possess characteristics associated with stem cells, specifically the ability to self renew and to propagate and give rise to all cell types found in a particular cancer sample. The term “cancer stem cell” (CSC) includes both gene alteration in stem cells (SCs) and gene alteration in a cell which becomes a CSC. In specific embodiments, the CSCs are breast CSCs, which are suitably CD24⁺ CD44⁺, illustrative examples of which include CD44^{high} CD24^{low}.

The term “catalytic activity” in relation to LSD1 is used herein to refer to the demethylation of a methylated lysine residue in a protein, such as a histone, especially histone 3.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In specific embodiments, the term “consisting essentially of”, in the context of a specific amino acid sequence disclosed herein, includes within its scope about 1 to about 50 optional amino acids (and all integer optional amino acids in between) upstream of the specific amino acid sequence and/or about 1 to about 50 optional amino acids (and all integer optional amino acids in between) downstream of the specific amino acid sequence.

By “corresponds to” or “corresponding to” is meant a sequence, such as a nucleic acid or amino acid sequence, that displays substantial sequence identity to a reference sequence (e.g. at least about 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g. at least 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

By “derivative” is meant a molecule, such as a polypeptide, that has been derived from the basic molecule by modification, for example by conjugation or complexing with other chemical moieties or by post-translational modification techniques as would be understood in the art. The term “derivative” also includes within its scope alterations that have been made to a parent sequence including additions or deletions that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

As used herein, the term “effector T cell” includes T cells which function to eliminate antigen (e.g. by producing cytokines which modulate the activation of other cells or by cytotoxic activity).

To enhance an immune response (“immunoenhancement”), as is well-known in the art, means to increase the animal's capacity to respond to foreign or disease-specific antigens (e.g. cancer antigens) i.e. those cells primed to attack such antigens are increased in number, activity, and ability to detect and destroy the those antigens. Strength of immune response is measured by standard tests including: direct measurement of peripheral blood lymphocytes by means known to the art; natural killer cell cytotoxicity assays (see, e.g., Provinciali et al. (1992) J. Immunol. Meth., 155: 19-24), cell proliferation assays (see, e.g., Vollenweider and Groseurth (1992) J. Immunol. Meth., 149: 133-135), immunoassays of immune cells and subsets (see, e.g., Loeffler et al. (1992) Cytom., 13: 169-174; Rivoltini et al. (1992) Can. Immunol. Immunother., 34: 241-251); or skin tests for cell-mediated immunity (see, e.g., Chang et al. (1993) Cancer Res., 53: 1043-1050). Any statistically significant increase in strength of immune response as measured by the foregoing tests is considered “enhanced immune response” or “immunoenhancement” as used herein. Enhanced immune response is also indicated by physical manifestations such as fever and inflammation, as well as healing of systemic and local infections, and reduction of symptoms in disease, i.e., decrease in tumor size, alleviation of symptoms of a disease or condition including, but not restricted to, leprosy, tuberculosis, malaria, naphthous ulcers, herpetic and papillomatous warts, gingivitis, artherosclerosis, the concomitants of AIDS such as Kaposi's sarcoma, bronchial infections, and the like. Such physical manifestations also define “enhanced immune response” or “immunoenhancement” as used herein.

As used herein, the term “epithelial-to-mesenchymal transition” (EMT) refers to the conversion from an epithelial cell to a mesenchymal phenotype, which is a normal process of embryonic development. EMT is also the process whereby injured epithelial cells that function as ion and fluid transporters become matrix remodeling mesenchymal cells, in carcinomas, this transformation typically results in altered cell morphology, the expression of mesenchymal proteins and increased invasiveness. The criteria for defining EMT in vitro involve the loss of epithelial cell polarity, the separation into individual cells and subsequent dispersion after the acquisition of cell motility (refer to Vincent-Salomon and Thiery (2003), Breast Cancer Res., 5(2): 101-6). Classes of molecules that change in expression, distribution and/or function during EMT, and that are causally involved, include growth factors (e.g. transforming growth factor (TGF)-β, wnts), transcription factors (e.g. SNAI, SMAD, LEF and nuclear β-catenin), molecules of the cell-to-cell adhesion axis (cadherins, catenins), cytoskeletal modulators (Rho family) and extracellular proteases (matrix metalloproteinases, plasminogen activators) (refer to Thompson and Newgreen, Cancer Res. 2005; 65(14):5991-5).

As used herein, the term “epithelium” refers to the covering of internal and external surfaces of the body, including the lining of vessels and other small cavities. It consists of a collection of epithelial cells forming a relatively thin sheet or layer due to the constituent cells being mutually and extensively adherent laterally by cell-to-cell junctions. The layer is polarized and has apical and basal sides. Despite the tight regimentation of the epithelial cells, the epithelium does have some plasticity and cells in an epithelial layer can alter shape, such as change from flat to columnar or pinch in at one end and expand at the other. However, these tend to occur in cell groups rather than individually (refer to Thompson and Newgreen, Cancer Res. 2005; 65(14):5991-5).

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

By “expression vector” is meant any genetic element capable of directing the transcription of a polynucleotide contained within the vector and suitably the synthesis of a peptide or polypeptide encoded by the polynucleotide. Such expression vectors are known to practitioners in the art.

The term “group” as applied to chemical species refers to a set of atoms that forms a portion of a molecule. In some instances, a group can include two or more atoms that are bonded to one another to form a portion of a molecule. A group can be monovalent or polyvalent (e.g. bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e. H⁺) added, and a negatively charged group can be envisioned as a neutral group with one or more protons removed. Non-limiting examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups. Groups such as alkyl, alkenyl, alkynyl, aryl, and heterocyclyl, whether used alone or in a compound word or in the definition of a group may be optionally substituted by one or more substituents. “Optionally substituted”, as used herein, refers to a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, phenylamino, diphenylamino, benzylamino, dibenzylamino, hydrazino, acyl, acylamino, diacylamino, acyloxy, heterocyclyl, heterocycloxy, heterocyclylamino, haloheterocyclyl, carboxy ester, carboxy, carboxy amide, mercapto, alkylthio, benzylthio, acylthio and phosphorus-containing groups. As used herein, the term “optionally substituted” may also refer to the replacement of a CH₂ group with a carbonyl (C═O) group. Non-limiting examples of optional substituents include alkyl, preferably C₁₋₈ alkyl (e.g. C₁₋₆ alkyl such as methyl, ethyl, propyl, butyl, cyclopropyl, cyclobutyl, cyclopentyl or cyclohexyl), hydroxy C₁₋₈ alkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc.), C₁₋₈ alkoxy, (e.g. C₁₋₆ alkoxy such as methoxy, ethoxy, propoxy, butoxy, cyclopropoxy, cyclobutoxy), halo (fluoro, chloro, bromo, iodo), monofluoromethyl, monochloromethyl, monobromomethyl, difluoromethyl, dichloromethyl, dibromomethyl, trifluoromethyl, trichloromethyl, tribromomethyl, hydroxy, phenyl (which itself may be further substituted, by an optional substituent as described herein, e.g., hydroxy, halo, methyl, ethyl, propyl, butyl, methoxy, ethoxy, acetoxy, amino), benzyl (wherein the CH₂ and/or phenyl group may be further substituted as described herein), phenoxy (wherein the CH₂ and/or phenyl group may be further substituted as described herein), benzyloxy (wherein the CH₂ and/or phenyl group may be further substituted as described herein), amino, C₁₋₈ alkylamino (e.g. C₁₋₆ alkyl, such as methylamino, ethylamino, propylamino), diC₁₋₈ alkylamino (e.g. C₁₋₆ alkyl, such as dimethylamino, diethylamino, dipropylamino), acylamino (e.g. NHC(O)CH₃), phenylamino (wherein phenyl itself may be further substituted as described herein), nitro, formyl, —C(O)—C₁₋₈ alkyl (e.g. C₁₋₆ alkyl, such as acetyl), O—C(O)-alkyl (e.g. C₁₋₆ alkyl, such as acetyloxy), benzoyl (wherein the CH₂ and/or phenyl group itself may be further substituted), replacement of CH₂ with C═O, CO₂H, CO₂C₁₋₈ alkyl (e.g. C₁₋₆ alkyl such as methyl ester, ethyl ester, propyl ester, butyl ester), CO₂phenyl (wherein phenyl itself may be further substituted), CONH₂, CONHphenyl (wherein phenyl itself may be further substituted as described herein), CONHbenzyl (wherein the CH₂ and/or phenyl group may be further substituted as described herein), CONH C₁₋₈ alkyl (e.g. C₁₋₆ alkyl such as methyl amide, ethyl amide, propyl amide, butyl amide), CONH C₁₋₈ alkylamine (e.g. C₁₋₆ alkyl such as aminomethyl amide, aminoethyl amide, aminopropyl amide, aminobutyl amide), —C(O)heterocyclyl (e.g. —C(O)-1-piperidine, —C(O)-1-piperazine, —C(O)-4-morpholine), —C(O)heteroaryl (e.g. —C(O)-1-pyridine, —C(O)-1-pyridazine, —C(O)-1-pyrimidine, —C(O)-1-pyrazine), CONHdiC₁₋₈ alkyl (e.g. C₁₋₆ alkyl).

“Heteroaralkyl” group means alkyl as defined above which is substituted with a heteroaryl group, e.g., —CH₂pyridinyl, —(CH₂)₂pyrimidinyl, —(CH₂)₃imidazolyl, and the like, and derivatives thereof.

The term “heteroaryl” or “heteroaromatic”, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, bezofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl.

Further examples of “heterocyclyl” and “heteroaryl” include, but are not limited to, the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazoyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, aziridinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

As used herein, “heteroarylene” refers to a bivalent monocyclic or multicyclic ring system, preferably of about 3 to about 15 members where one or more, more preferably 1 to 3 of the atoms in the ring system is a heteroatom, that is, an element other than carbon, for example, nitrogen, oxygen and sulfur atoms. The heteroarylene group may be optionally substituted with one or more, preferably 1 to 3, aryl group substituents. Exemplary heteroarylene groups include, for example, 1,4-imidazolylene.

The term “heterocycle”, “heteroaliphatic” or “heterocyclyl” as used herein is intended to mean a 5- to 10-membered nonaromatic heterocycle containing from 1 to 4 heteroatoms selected from the group consisting of O, N and S, and includes bicyclic groups.

“Heterocyclylalkyl” group means alkyl as defined above which is substituted with a heterocycle group, e.g., —CH₂pyrrolidin-1-yl, —(CH₂)₂piperidin-1-yl, and the like, and derivatives thereof.

The term “high”, as used herein, refers to a measure that is greater than normal, greater than a standard such as a predetermined measure or a subgroup measure or that is relatively greater than another subgroup measure. For example, CD44^{high} refers to a measure of CD44 that is greater than a normal CD44 measure. Consequently, “CD44^{high}” always corresponds to, at the least, detectable CD44 in a relevant part of a subject's body or a relevant sample from a subject's body. A normal measure may be determined according to any method available to one skilled in the art. The term “high” may also refer to a measure that is equal to or greater than a predetermined measure, such as a predetermined cutoff. If a subject is not “high” for a particular marker, it is “low” for that marker. In general, the cut-off used for determining whether a subject is “high” or “low” should be selected such that the division becomes clinically relevant.

The term “hormone receptor negative (HR−) tumor” means a tumor that does not express a receptor for a hormone that stimulates the proliferation, survival or viability of the tumor above a certain threshold as determined by standard methods (e.g. immunohistochemical staining of nuclei in the patients biological samples). The threshold may be measured, for example, using an Allred score or gene expression. See, e.g. Harvey et al. (1999, J Clin Oncol, 17: 1474-1481) and Badve et al. (2008, J Clin Oncol, 26(15): 2473-2481). In some embodiments, the tumor does not express an estrogen receptor (ER−) and/or a progesterone receptor (PR−).

The term “hormone receptor positive (HR+) tumor” means a tumor that expresses a receptor for a hormone that stimulates the proliferation, survival or viability of the tumor above a certain threshold as determined by standard methods (e.g. immunohistochemical staining of nuclei in the patients biological samples). The threshold may be measured, for example, using an Allred score or gene expression. See, e.g., Harvey et al. (1999, J Clin Oncol, 17: 1474-1481) and Badve et al. (2008, J Clin Oncol, 26(15): 2473-2481). In some embodiments, the tumor expresses an estrogen receptor (ER) and/or a progesterone receptor (PR).

The term “host cell” includes an individual cell or cell culture which can be or has been a recipient of any recombinant vector(s) or isolated polynucleotide of the invention. Host cells include progeny of a single host cell and the progeny may not necessarily be completely identical (in morphology or in total DNA complement) to the original parent cell due to natural, accidental or deliberate mutation and/or change. A host cell includes cells transfected or infected in vivo or in vitro with a recombinant vector or a polynucleotide of the invention. A host cell which comprises a recombinant vector of the invention is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The term “hydrocarbyl” as used herein includes any radical containing carbon and hydrogen including saturated, unsaturated, aromatic, straight or branched chain or cyclic including polycyclic groups. Hydrocarbyl includes but is not limited to C₁-C₈alkyl, C₂-C₈alkenyl, C₂-C₈alkynyl, C₃-C₁₀cycloalkyl, aryl such as phenyl and naphthyl, Ar(C₁-C₈)alkyl such as benzyl, any of which may be optionally substituted.

Reference herein to “immuno-interactive” includes reference to any interaction, reaction, or other form of association between molecules and in particular where one of the molecules is, or mimics, a component of the immune system.

The term “inhibitor” as used herein refers to an agent that decreases or inhibits at least one function or biological activity of a target molecule. For example, an LSD1 inhibitor may decrease or reduce at least one function or biological activity of LSD1. LSD1 inhibitors may inhibit or reduce the nuclear translocation of LSD1, may inhibit or reduce the catalytic activity of LSD1, may inhibit or reduce the phosphorylation of LSD1, and/or may inhibit or reduce the expression of LSD1. In particular embodiments, the term “LSD1 inhibitor” refers to an agent that inhibits the nuclear translocation of LSD1.

As used herein, the term “isolated” refers to material that is substantially or essentially free from components that normally accompany it in its native state. For example, an “isolated proteinaceous molecule” refers to in vitro isolation and/or purification of a proteinaceous molecule from its natural cellular environment and from association with other components of the cell. “Substantially free” means that a preparation of proteinaceous molecule is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of proteinaceous molecule has less than about 30, 25, 20, 15, 10, 9, 8, 7, 6, 5, 4, 3, 2 or 1% (by dry weight), of molecules that are not the subject of this invention (also referred to herein as “contaminating molecules”). When the proteinaceous molecule is recombinantly produced, it is also desirably substantially free of culture medium, i.e., culture medium represents less than about 20, 15, 10, 5, 4, 3, 2 or 1% of the volume of the preparation. The invention includes isolated or purified preparations of at least 0.01, 0.1, 1.0, and 10 milligrams in dry weight.

The term “lower alkyl” refers to straight and branched chain alkyl groups having from 1 to 6 carbon atoms, such as methyl, ethyl, n-propyl, i-propyl, n-butyl, t-butyl, sec-butyl, n-pentyl, n-hexyl, 2-methylpentyl, and the like. In some embodiments, the lower alkyl group is methyl or ethyl.

The term “lower alkoxy” refers to straight and branched chain alkoxy groups having from 1 to 6 carbon atoms, such as methoxy, ethoxy, n-propoxy, i-propoxy, n-butoxy, t-butoxy, sec-butoxy, n-pentoxy, n-hexoxy, 2-methyl-pentoxy, and the like. Usually, the lower alkoxy group is methoxy or ethoxy.

As used herein, the term “LSD1 overexpressing cell” refers to a vertebrate cell, particularly a mammalian or avian cell, especially a mammalian cell, that expresses LSD1 at a detectably greater level than a normal cell. The cell may be a vertebrate cell, such as a primate cell; an avian cell; a livestock animal cell such as a sheep cell, cow cell, horse cell, deer cell, donkey cell and pig cell; a laboratory test animal cell such as a rabbit cell, mouse cell, rat cell, guinea pig cell and hamster cell; a companion animal cell such as a cat cell and dog cell; and a captive wild animal cell such as a fox cell, deer cell and dingo cell. In particular embodiments, the LSD1 overexpressing cell is a human cell. In specific embodiments, the LSD1 overexpressing cell is a cancer stem cell or a non-cancer stem cell tumor cell; preferably a cancer stem cell tumor cell. Overexpression can also be by 10%, 20%, 30%, 40%, 50%, 60%, 70%, 80%, 90% or more in comparison to a normal cell or comparison cell (e.g. a breast cell).

As used herein, the term “mesenchymal-to-epithelial transition” (MET) is a reversible biological process that involves the transition from motile, multipolar or spindle-shaped mesenchymal cells to planar arrays of polarized cells called epithelia. MET is the reverse process of EMT. METs occur in normal development, cancer metastasis and induced pluripotent stem cell reprogramming.

As used herein, the term “mesenchyme” refers to the part of the embryonic mesoderm, consisting of loosely packed, unspecialized cells set in a gelatinous ground substance, from which connective tissue, bone, cartilage and the circulatory and lymphatic systems develop. Mesenchyme is a collection of cells which form a relatively diffuse tissue network. Mesenchyme is not a complete cellular layer and the cells typically have only points on their surface engaged in adhesion to their neighbors. These adhesions may also involve cadherin association (see Thompson and Newgreen (2005), Cancer Res., 65(14): 5991-5).

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

As used herein, the terms “overexpress”, “overexpression”, “overexpressing” or “overexpressed” interchangeably refer to a gene (e.g. LSD1 gene or PKC gene) that is transcribed or translated at a detectably greater level, usually in a cancer cell, in comparison to a normal cell. Overexpression, therefore, refers to both overexpression of protein and RNA (due to increased transcription, post transcriptional processing, translation, post translational processing, altered stability and altered protein degradation), as well as local overexpression due to altered protein traffic patterns (increased nuclear localization) and augmented functional activity, for example, as in an increased enzyme hydrolysis of substrate. Overexpression can also be by 10%, 20%, 30%, 40%, 50%, 60%, 70%, 80%, 90% or more in comparison to a normal cell or comparison cell (e.g. a breast cell).

The term “operably linked” as used herein means placing a structural gene under the regulatory control of a regulatory element including, but not limited to, a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting, i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting, i.e. the genes from which it is derived.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

“Phenylalkyl” means alkyl as defined above which is substituted with phenyl, e.g., —CH₂phenyl, —(CH₂)₂phenyl, —(CH₂)₃phenyl, CH₃CH(CH₃)CH₂phenyl, and the like and derivatives thereof. Phenylalkyl is a subset of the aralkyl group.

The term “phosphorylating activity” is used herein to refer to the ability of a PKC to phosphorylate a hydroxyl group on a protein substrate, especially a hydroxyl group on a serine or threonine residue on a protein substrate.

As used herein, the term “PKC overexpressing cell” refers to a vertebrate cell, particularly a mammalian or avian cell, especially a mammalian cell, that expresses PKC, especially PKC-0, at a detectably greater level than a normal cell. The cell may be a vertebrate cell, such as a primate cell; an avian cell; a livestock animal cell such as a sheep cell, cow cell, horse cell, deer cell, donkey cell and pig cell; a laboratory test animal cell such as a rabbit cell, mouse cell, rat cell, guinea pig cell and hamster cell; a companion animal cell such as a cat cell and dog cell; and a captive wild animal cell such as a fox cell, deer cell and dingo cell. In particular embodiments, the PKC overexpressing cell is a human cell. In specific embodiments, the PKC overexpressing cell is a cancer stem cell or a non-cancer stem cell tumor cell; preferably a cancer stem cell tumor cell. Overexpression can also be by 10%, 20%, 30%, 40%, 50%, 60%, 70%, 80%, 90% or more in comparison to a normal cell or comparison cell (e.g. a breast cell).

As used herein, the terms “polypeptide”, “proteinaceous molecule”, “peptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogues of an amino acid including, for example, unnatural amino acids or polypeptides with substituted linkages.

As used herein, the terms “prevent”, “prevented” or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

By “pro-inflammatory immune response” is meant an immune response that includes one or more of increased levels of pro-inflammatory cytokines (e.g. IL-6, IL-23, IL-17, IL-1α, IL-1β, and TNF-α) and/or increased levels of effector T cells, as compared to the levels of the one or more pro-inflammatory cytokines and/or the levels of effector T cells in a control, healthy mammal.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, fatigue, etc. In another embodiment, the reduction may be determined objectively, for example when the number of CSCs and/or non-CSC tumor cells in a sample from a patient is lower than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

The term “selective” and grammatical variants thereof are used herein to refer to agents that inhibit a target molecule without substantially inhibiting the function of another molecule. For example, an LSD1 selective inhibitor is an agent which inhibits LSD1 without substantially inhibiting the function of another LSD or another enzyme such as a monoamine oxidase (MAO). Generally an agent that is selective for LSD1 exhibits LSD1 selectivity of greater than about 2-fold, 5-fold, 10-fold, 20-fold, 50-fold or greater than about 100-fold with respect to inhibition of another LSD or MAO. In other embodiments, selective molecules display at least 50-fold greater inhibition towards LSD1 than another LSD or MAO. In further embodiments, selective molecules display at least 100-fold greater inhibition towards LSD1 than towards another LSD or MAO. In still further embodiments, selective molecules display at least 500-fold greater inhibition towards LSD1 than towards another LSD or MAO. In yet further embodiments, selective molecules display at least 100-fold greater inhibition towards LSD1 than towards another LSD or MAO.

As used herein, the terms “salts” and “prodrugs” include any pharmaceutically acceptable salt, ester, hydrate or any other compound which, upon administration to the recipient, is capable of providing (directly or indirectly) a proteinaceous molecule of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs can be carried out by methods known in the art. For example, metal salts can be prepared by reaction of a compound of the invention with a metal hydroxide. An acid salt can be prepared by reacting an appropriate acid with a proteinaceous molecule of the invention.

The term “stringency” as used herein, refers to the temperature and ionic strength conditions, and presence or absence of certain organic solvents during hybridization and washing procedures. The higher the stringency, the higher will be the degree of complementarity between immobilized target nucleotide sequences and the labelled probe polynucleotide sequences that remain hybridized to the target after washing. The term “high stringency” refers to temperature and ionic conditions under which only nucleotide sequences having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

The term “subject” as used herein refers to a vertebrate subject, particularly a mammalian or avian subject, for whom therapy or prophylaxis is desired. Suitable subjects include, but are not limited to, primates; avians; livestock animals such as sheep, cows, horses, deer, donkeys and pigs; laboratory test animals such as rabbits, mice, rats, guinea pigs and hamsters; companion animals such as cats and dogs; and captive wild animals such as foxes, deer and dingoes. In particular, the subject is a human. However, it will be understood that the aforementioned terms do not imply that symptoms are present.

As used herein, the terms “treatment”, “treating”, and the like, refer to obtaining a desired pharmacologic and/or physiologic effect. The effect may be therapeutic in terms of a partial or complete cure for a disease or condition (e.g. a hematologic malignancy) and/or adverse affect attributable to the disease or condition. These terms also cover any treatment of a condition or disease in a mammal, particularly in a human, and include: (a) inhibiting the disease or condition, i.e., arresting its development; or (b) relieving the disease or condition, i.e., causing regression of the disease or condition.

As used herein, the term “tumor” refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. The term “non-metastatic” refers to a cancer that is benign or that remains at the primary site and has not penetrated into the lymphatic or blood vessel system or to tissues other than the primary site. Generally, a non-metastatic cancer is any cancer that is a Stage 0, I or II cancer. By “early stage cancer” is meant a cancer that is not invasive or metastatic or is classified as a Stage 0, I or II cancer. The term “late stage cancer” generally refers to a Stage III or IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, retinoblastoma, melanoma, brain cancer, non-small cell lung cancer, squamous cell cancer of the head and neck, endometrial cancer, multiple myeloma, mesothelioma, rectal cancer and esophageal cancer. In an exemplary embodiment, the cancer is breast cancer.

As used herein, the term “vector” refers to a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e. a vector that exists as an extra-chromosomal entity, the replication of which is independent of chromosomal replication, e.g. a linear or closed circular plasmid, an extra-chromosomal element, a mini-chromosome or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in fungi, bacterial or animal cells, preferably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

Each embodiment described herein is to be applied mutatis mutandis to each and every embodiment unless specifically stated otherwise.

### 2. LSD1 Inhibitors

The present invention is based, in part, on the determination that inhibitors of LSD1 inhibit immune checkpoints, particularly PD-L1 and/or PD-L2. Accordingly, the inventors have conceived that LSD1 inhibitors may be used for a range of applications, including for enhancing an immune response in a subject to a target antigen by an immune-modulating agent or for the treatment of a cancer or an infection.

The LSD1 inhibitor includes and encompasses any active agent that reduces the accumulation, function or stability of LSD1; or decreases expression of the LSD1 gene; and such inhibitors include without limitation, small molecules and macromolecules such as nucleic acids, peptides, polypeptides, peptidomimetics, carbohydrates, polysaccharides, lipopolysaccharides, lipids or other organic (carbon containing) or inorganic molecules. In some embodiments, the LSD1 inhibitor is an inhibitor of the catalytic activity of LSD1 or an inhibitor of the nuclear translocation of LSD1.

In some embodiments, the LSD1 inhibitor selectively inhibits LSD1 over at least one other LSD or another enzyme such as an MAO. In some embodiments, the LSD1 inhibitor selectively inhibits LSD1 over the other LSD subtypes and MAOs. In some embodiments, the LSD1 inhibitor exhibits LSD1 selectivity of greater than about 2-fold, 5-fold, 10-fold, 20-fold, 50-fold or greater than about 100-fold with respect to inhibition of another LSD or MAO. In other embodiments, selective molecules display at least 50-fold greater inhibition towards LSD1 than another LSD or MAO. In further embodiments, selective molecules display at least 100-fold greater inhibition towards LSD1 than towards another LSD or MAO. In still further embodiments, selective molecules display at least 500-fold greater inhibition towards LSD1 than towards another LSD or MAO. In yet further embodiments, selective molecules display at least 100-fold greater inhibition towards LSD1 than towards another LSD or MAO. In some embodiments, the LSD1 inhibitor is a non-selective LSD1 inhibitor.

2.1 Nucleic Acid Molecules

In some embodiments, the LSD1 inhibitor is an antagonistic nucleic acid molecule that functions to inhibit the transcription or translation of LSD1 transcripts. Representative transcripts of this type include nucleotide sequences corresponding to any one the following sequences: (1) human LSD1 nucleotide sequences as set forth for example in GenBank Accession Nos. NM_015013.3, NP_001009999.1, and NM_001009999.2; (2) nucleotide sequences that share at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) nucleotide sequences that hybridize under at least low, medium or high stringency conditions to the sequences referred to in (1); (4) nucleotide sequences that encode any one of the following amino acid sequences: human LSD1 amino acid sequences as set forth for example in GenPept Accession Nos. NP_055828.2, NP_001009999.1 and O60341.2; (5) nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and nucleotide sequences that encode an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

Illustrative antagonist nucleic acid molecules include antisense molecules, aptamers, ribozymes and triplex forming molecules, RNAi and external guide sequences. The nucleic acid molecules can act as effectors, inhibitors, modulators, and stimulators of a specific activity possessed by a target molecule, or the functional nucleic acid molecules can possess a de novo activity independent of any other molecules.

Antagonist nucleic acid molecules can interact with any macromolecule, such as DNA, RNA, polypeptides, or carbohydrate chains. Thus, antagonist nucleic acid molecules can interact with LSD1 mRNA or the genomic DNA of LSD1 or they can interact with an LSD1 polypeptide. Often antagonist nucleic acid molecules are designed to interact with other nucleic acids based on sequence homology between the target molecule and the antagonist nucleic acid molecule. In other situations, the specific recognition between the antagonist nucleic acid molecule and the target molecule is not based on sequence homology between the antagonist nucleic acid molecule and the target molecule, but rather is based on the formation of tertiary structure that allows specific recognition to take place.

In some embodiments, anti-sense RNA or DNA molecules are used to directly block the translation of LSD1 by binding to targeted mRNA and preventing protein translation. Antisense molecules are designed to interact with a target nucleic acid molecule through either canonical or non-canonical base pairing. The interaction of the antisense molecule and the target molecule may be designed to promote the destruction of the target molecule through, for example, RNAseH mediated RNA-DNA hybrid degradation. Alternatively the antisense molecule may be designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Non-limiting methods include in vitro selection experiments and DNA modification studies using DMS and DEPC. In specific examples, the antisense molecules bind the target molecule with a dissociation constant (K_{d}) less than or equal to 10⁻⁶, 10⁻⁸, 10⁻¹⁰, or 10⁻¹². In specific embodiments, antisense oligodeoxyribonucleotides derived from the translation initiation site, e.g., between −10 and +10 regions are employed.

Aptamers are molecules that interact with a target molecule, suitably in a specific way. Aptamers are generally small nucleic acids ranging from 15-50 bases in length that fold into defined secondary and tertiary structures, such as stem-loops or G-quartets. Aptamers can bind small molecules, such as ATP and theophylline, as well as large molecules, such as reverse transcriptase and thrombin. Aptamers can bind very tightly with K_{d}s from the target molecule of less than 10⁻¹² M. Suitably, the aptamers bind the target molecule with a K_{d} less than 10⁻⁶, 10⁻⁸, 10^{-1°}, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule. It is desirable that an aptamer have a K_{d} with the target molecule at least 10-, 100-, 1000-, 10,000-, or 100,000-fold lower than the K_{d} with a background-binding molecule. A suitable method for generating an aptamer to a target of interest (e.g., PHD, FIH-1 or vHL) is the “Systematic Evolution of Ligands by EXponential Enrichment” (SELEX™). The SELEX™ method is described in U.S. Pat. Nos. 5,475,096 and 5,270,163 (see also WO 91/19813). Briefly, a mixture of nucleic acids is contacted with the target molecule under conditions favorable for binding. The unbound nucleic acids are partitioned from the bound nucleic acids, and the nucleic acid-target complexes are dissociated. Then the dissociated nucleic acids are amplified to yield a ligand-enriched mixture of nucleic acids, which is subjected to repeated cycles of binding, partitioning, dissociating and amplifying as desired to yield highly specific high affinity nucleic acid ligands to the target molecule.

In other embodiments, anti-LSD1 ribozymes are used for catalyzing the specific cleavage of LSD1 RNA. The mechanism of ribozyme action involves sequence specific hybridization of the ribozyme molecule to complementary target RNA, followed by a endonucleolytic cleavage. There are several different types of ribozymes that catalyze nuclease or nucleic acid polymerase type reactions, which are based on ribozymes found in natural systems, such as hammerhead ribozymes, hairpin ribozymes, and tetrahymena ribozymes. There are also a number of ribozymes that are not found in natural systems, but which have been engineered to catalyze specific reactions de novo. Representative ribozymes cleave RNA or DNA substrates. In some embodiments, ribozymes that cleave RNA substrates are employed. Specific ribozyme cleavage sites within potential RNA targets are initially identified by scanning the target molecule for ribozyme cleavage sites, which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base pairing. Triplex molecules are preferred because they can bind target regions with high affinity and specificity. It is generally desirable that the triplex forming molecules bind the target molecule with a K_{d} less than 10⁻⁶, 10⁻⁸, 10^{-1°}, or 10⁻¹².

External guide sequences (EGSs) are molecules that bind a target nucleic acid molecule forming a complex, and this complex is recognized by RNAse P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. Similarly, eukaryotic EGS/RNAse P-directed cleavage of RNA can be utilized to cleave desired targets within eukaryotic cells.

In other embodiments, RNA molecules that mediate RNA interference (RNAi) of an LSD1 gene or LSD1 transcript can be used to reduce or abrogate gene expression. RNAi refers to interference with or destruction of the product of a target gene by introducing a single-stranded or usually a double-stranded RNA (dsRNA) that is homologous to the transcript of a target gene. RNAi methods, including double-stranded RNA interference (dsRNAi) or small interfering RNA (siRNA), have been extensively documented in a number of organisms, including mammalian cells and the nematode C. elegans (Fire et al. (1998) Nature, 391: 806-811). In mammalian cells, RNAi can be triggered by 21- to 23-nucleotide (nt) duplexes of small interfering RNA (siRNA) (Chiu et al. (2002) Mol. Cell., 10: 549-561; Elbashir et al. (2001) Nature, 411: 494-498), or by micro-RNAs (miRNA), functional small-hairpin RNA (shRNA), or other dsRNAs which are expressed in vivo using DNA templates with RNA polymerase III promoters (Zeng et al. (2002) Mol. Cell, 9: 1327-1333; Paddison et al. (2002) Genes Dev., 16: 948-958; Lee et al. (2002) Nature Biotechnol., 20: 500-505; Paul et al. (2002) Nature Biotechnol., 20: 505-508; Tuschl (2002) Nature Biotechnol., 20: 440-448; Yu et al. (2002) Proc. Natl. Acad. Sci. USA, 99(9): 6047-6052; McManus et al. (2002) RNA, 8: 842-850; Sui et al. (2002) Proc. Natl. Acad. Sci. USA, 99(6): 5515-5520).

In specific embodiments, dsRNA per se and especially dsRNA-producing constructs corresponding to at least a portion of an LSD1 gene are used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for an LSD1 gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts, which then anneal to form a dsRNA having homology to a target gene.

Absolute homology is not required for RNAi, with a lower threshold being described at about 85% homology for a dsRNA of about 200 base pairs (Plasterk and Ketting (2000) Current Opinion in Genetics and Dev., 10: 562-67). Therefore, depending on the length of the dsRNA, the RNAi-encoding nucleic acids can vary in the level of homology they contain toward the target gene transcript, i.e., with dsRNAs of 100 to 200 base pairs having at least about 85% homology with the target gene, and longer dsRNAs, i.e., 300 to 100 base pairs, having at least about 75% homology to the target gene. RNA-encoding constructs that express a single RNA transcript designed to anneal to a separately expressed RNA, or single constructs expressing separate transcripts from convergent promoters, are suitably at least about 100 nucleotides in length. RNA-encoding constructs that express a single RNA designed to form a dsRNA via internal folding are usually at least about 200 nucleotides in length.

The promoter used to express the dsRNA-forming construct may be any type of promoter if the resulting dsRNA is specific for a gene product in the cell lineage targeted for destruction. Alternatively, the promoter may be lineage specific in that it is only expressed in cells of a particular development lineage. This might be advantageous where some overlap in homology is observed with a gene that is expressed in a non-targeted cell lineage. The promoter may also be inducible by externally controlled factors, or by intracellular environmental factors.

In some embodiments, RNA molecules of about 21 to about 23 nucleotides, which direct cleavage of specific mRNA to which they correspond, as for example described by Tuschl et al. in U.S. 2002/0086356, can be utilized for mediating RNAi. Such 21- to 23-nt RNA molecules can comprise a 3′ hydroxyl group, can be single-stranded or double-stranded (as two 21- to 23-nt RNAs) wherein the dsRNA molecules can be blunt ended or comprise overhanging ends (e.g. 5′, 3′).

In some embodiments, the antagonist nucleic acid molecule is a siRNA. siRNAs can be prepared by any suitable method. For example, reference may be made to International Publication WO 02/44321, which discloses siRNAs capable of sequence-specific degradation of target mRNAs when base-paired with 3′ overhanging ends, which is incorporated by reference herein. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER™ siRNA Construction Kit.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US 2002/0120129, each of which is incorporated herein by reference.

Illustrative RNAi molecules (e.g. LSD1 siRNA and shRNA) are described in the art (e.g. Yang et al. (2010) Proc. Natl. Acad. Sci. USA, 107: 21499-21504; and He et al. (2012) Transcription, 3: 1-16) or available commercially from Santa Cruz Biotechnology, Inc. (Santa Cruz, Calif., USA) and OriGene Technologies, Inc. (Rockville, Md., USA).

2.2 Polypeptide or Peptide Based Molecules

The present invention further contemplates peptide or polypeptide based inhibitor compounds. For example, BHC80 (also known as PHD finger protein 21A) forms part of a complex with LSD1 and can inhibit LSD1 demethylase activity. Accordingly, the present invention further contemplates the use of BHC80 or biologically active fragments thereof for inhibiting LSD1 catalytic activity. Amino acid sequences of BHC80 polypeptides, and nucleotide sequences encoding BHC80 polypeptides, are publicly available. In this regard, reference may be made for example to GenBank Accession No. NP057705 for a Homo sapiens BHC80 amino acid sequence; and GenBank NM016621 for a nucleotide sequence encoding the amino acid sequence set forth in GenBank Accession No. NP057705; 2) GenBank Accession No. NP620094 for a Mus musculus BHC80 amino acid sequence; and GenBank NM138755 for a nucleotide sequence encoding the amino acid sequence set forth in GenBank Accession No. NP620094; 3) GenBank Accession No. NP00118576.1 for a Gallus gallus BHC80 amino acid sequence; and GenBank NM001199647 for a nucleotide sequence encoding the amino acid sequence set forth in GenBank Accession No. NP00118576.1; and 4) GenBank Accession No. DAA21793 for a Bos taurus BHC80 amino acid sequence.

Illustrative BHC80 polypeptides are selected from the group consisting of: (1) a polypeptide comprising an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with the amino acid sequence listed in any one of the GenBank BHC80 polypeptide entries noted above; (2) a polypeptide comprising an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with the amino acid sequence listed in any one of the GenBank BHC80 polypeptide entries noted above; (3) a polypeptide comprising an amino acid sequence that is encoded by a nucleotide sequence that hybridizes under at least low, medium or high stringency conditions to the nucleotide sequence listed in any one of the GenBank BHC80 polynucleotide entries noted above; (4) a polypeptide comprising an amino acid sequence that is encoded by a nucleotide sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity to the nucleotide sequence listed in any one of the GenBank BHC80 polynucleotide entries noted above; and (5) a fragment of a polypeptide according to any one of (1) to (4), which inhibits LSD1 catalytic activity.

A BHC80 polypeptide can be introduced into a cell by delivering a polypeptide per se, or by introducing into the cell a BHC80 nucleic acid comprising a nucleotide sequence encoding a BHC80 polypeptide. In some embodiments, a BHC80 nucleic acid comprises a nucleotide sequence selected from: (1) a BHC80 nucleotide sequence listed in any one of the GenBank BHC80 polynucleotide entries noted above; (2) a nucleotide sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (1); (3) a nucleotide sequence that hybridizes under at least low, medium or high stringency conditions to the sequences referred to in (1); (4) a nucleotide sequence that encodes an amino acid sequence listed in any one of the GenBank BHC80 polypeptide entries noted above; (5) a nucleotide sequence that encodes an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence similarity with any one of the sequences referred to in (4); and a nucleotide sequence that encodes an amino acid sequence that shares at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% sequence identity with any one of the sequences referred to in (4).

The BHC80 nucleic acid can be in the form of a recombinant expression vector. The BHC80 nucleotide sequence can be operably linked to a transcriptional control element(s), e.g. a promoter, in the expression vector. Suitable vectors include, for example, recombinant retroviruses, lentiviruses, and adenoviruses; retroviral expression vectors, lentiviral expression vectors, nucleic acid expression vectors, and plasmid expression vectors. In some cases, the expression vector is integrated into the genome of a cell. In other cases, the expression vector persists in an episomal state in a cell.

Suitable expression vectors include, but are not limited to, viral vectors (e.g., viral vectors based on vaccinia virus; poliovirus; adenovirus (see, e.g., Li et al. (1994) Invest Ophthalmol Vis Sci, 35: 2543-2549; Borras et al. (1999) Gene Ther, 6:515-524; Li and Davidson (1995) PNAS, 92: 7700-7704; Sakamoto et al. (1999) H Gene Ther, 5: 1088-1097; WO 94/12649, WO 93/03769; WO 93/19191; WO 94/28938; WO 95/11984 and WO 95/00655); adeno-associated virus (see, e.g., Ali et al. (1998) Hum Gene Ther, 9: 8186; Flannery et al. (1997) PNAS, 94: 6916-6921; Bennett et al. (1997) Invest Ophthalmol Vis Sci, 38: 2857-2863; Jomary et al. (1997) Gene Ther, 4: 683-690; Rolling et al. (1999) Hum Gene Ther, 10: 641-648; Ali et al. (1996) Hum Mol Genet., 5: 591-594; Srivastava in WO 93/09239; Samulski et al. (1989) J. Vir., 63: 3822-3828; Mendelson et al. (1988) Virol., 166: 154-165; and Flotte et al. (1993) PNAS, 90:10613-10617); SV40; herpes simplex virus; human immunodeficiency virus (see, e.g., Miyoshi et al. (1997) PNAS, 94: 10319-23; Takahashi et al. (1999) J Virol, 73: 7812-7816); a retroviral vector (e.g. Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus and mammary tumor virus); and the like.

2.3 Small Molecules

The present invention also contemplates small molecule agents that reduce the catalytic activity of LSD1.

Small molecule agents that reduce the catalytic activity of LSD1 that are suitable for use in the present invention include MAO inhibitors that also inhibit LSD1 catalytic activity; polyamine compounds that inhibit LSD1 catalytic activity; phenylcyclopropylamine derivatives that inhibit LSD1 catalytic activity; and the like.

Non-limiting examples of MAO inhibitors include MAO-A-selective inhibitors, MAO-B-selective inhibitors, and MAO non-selective inhibitors. Illustrative examples of MAO inhibitors include reported inhibitors of the MAO-A isoform, which preferentially deaminates 5-hydroxytryptamine (serotonin) (5-HT) and norepinephrine (NE), and/or the MAO-B isoform, which preferentially deaminates phenylethylamine (PEA) and benzylamine (both MAO-A and MAO-B metabolize Dopamine (DA)). In various embodiments, MAO inhibitors may be irreversible or reversible (e.g. reversible inhibitors of MAO-A (RIMA)), and may have varying potencies against MAO-A and/or MAO-B (e.g. non-selective dual inhibitors or isoform-selective inhibitors).

In some embodiments, the MAO inhibitors are selected from: clorgyline; L-deprenyl; isocarboxazid (Marplan™); ayahuasca; nialamide; iproniazide; iproclozide; moclobemide (Aurorix™; 4-chloro-N-(2-morpholin-4-ylethyl)benzamide); phenelzine (Nardil™; (+)-2-phenylethylhydrazine); tranylcypromine (Parnate™; (+)-trans-2-phenylcyclopropan-1-amine) (the congeneric of phenelzine); toloxatone; levo-deprenyl (Selegiline™); harmala; RIMAs (e.g., moclobemide, described in Da Prada et al. (1989), J Pharmacol Exp Ther, 248: 400-414); brofaromine; and befloxatone, described in Curet et al. (1998), J Affect Disord, 51: 287-30), lazabemide (Ro 19 6327), described in Ann. Neurol., 40(1): 99-107 (1996), and SL25.1131 (3(S),3a(S)-3-methoxymethyl-7-[4,4,4-trifluorobutoxy]-3,3a,4,5-tetrahydro-1,3-oxazolo[3,4-a]quinolin-1-one), described in Aubin et al. (2004). J. Pharmacol. Exp. Ther., 310: 1171-1182); selegiline hydrochloride (1-deprenyl, ELDEPRYL, ZELAPAR); dimethylselegilene; safinamide; rasagiline (AZILECT); bifemelane; desoxypeganine; harmine (also known as telepathine or banasterine); linezolid (ZYVOX, ZYVOXID); pargyline (EUDATIN, SUPIRDYL); dienolide kavapyrone desmethoxyyangonin; 5-(4-Arylmethoxyphenyl)-2-(2-cyanoethyl)tetrazoles; NCD36 (2-[N-(4-phenylbenzenecarbonyl)]amino-6-(trans-2-phenylcyclopropan-1-amino)-N-(3-methylbenzyl)hexanamide hydrochloride; and the like.

Small molecule LSD1 inhibitors may also be selected from polyamine compounds as described for example by Woster et al. in U.S. Publication No. 2007/0208082, which is incorporated herein by reference in its entirety. Illustrative polyamine inhibitors of LSD1 include compounds according to formula (II):

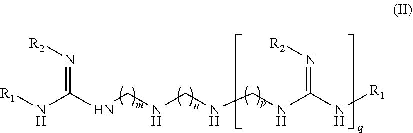

or a salt, solvate, or hydrate thereof, where n is an integer from 1 to 12; m and p are independently an integer from 1 to 5; q is 0 or 1; each R₁ is independently selected from the group consisting of C₁-C₈ alkyl, C₄-C₁₅ cycloalkyl, C₃-C₁₅ branched alkyl, C₆-C₂₀ aryl, C₆-C₂₀ heteroaryl, C₇-C₂₄ aralkyl, C₇-C₂₄ heteroaralkyl, and

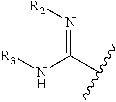

where R₃ is selected from the group consisting of C₁-C₈ alkyl, C₄-C₁₅ cycloalkyl, C₃-C₁₅ branched alkyl, C₆-C₂₀ aryl, C₆-C₂₀ heteroaryl, C₇-C₂₄ aralkyl and C₇-C₂₄ heteroaralkyl; and
each R₂ is independently selected from hydrogen or a C₁-C₈ alkyl.

A suitable polyamine compound is a compound of Formula (II), wherein one or both R1 is a C6-C20 aryl, such as a single ring aryl, including without limitation, a phenyl. In one embodiment, the compound is of the formula (II) and each R1 is phenyl. In one embodiment, q is I, m and p are 3, and n is 4. In another embodiment, q is l, m and p are 3, and n is 7.

A suitable polyamine compound is a compound of Formula (II), where at least one or both R₁ is a C₈-C₁₂ or a C₁-C₈ alkyl, such as a linear alkyl. One or both R₁ may be a C₁-C₈ linear alkyl, such as methyl or ethyl. In one embodiment, each R₁ is methyl. One or both R₁ may comprise or be a C₄-C₁₅ cycloalkyl group, such as a cycloalkyl group containing a linear alkyl group, where the cycloalkyl group is connected to the molecule either via its alkyl or cycloalkyl moiety. For instance, one or both R₁ may be cyclopropylmethyl or cyclohexylmethyl. In one embodiment, one R₁ is cyclopropylmethyl or cyclohexylmethyl and the other R₁ is a linear alkyl group, such as a linear C₁-C₈ unsubstituted alkyl group, including without limitation an ethyl group. In one embodiment, R₁ is a C₃-C₁₅ branched alkyl group such as isopropyl. When R₁ is a C₁-C₈ substituted alkyl, the substituted alkyl may be substituted with any substituent, including a primary, secondary, tertiary or quaternary amine. Accordingly, in one embodiment, R₁ is a C₁-C₈ alkyl group substituted with an amine such that R₁ may be e.g., alkyl-NH₂ or an alkyl-amine-alkyl moiety such as —(CH₂)_{y}NH(CH₂)_{z}CH₃ where y and z are independently an integer from 1 to 8. In one embodiment, R₁ is —(CH₂)₃NH₂.

In one embodiment, the compound is of the formula (II) where one or both R₁ is a C₇-C₂₄ substituted or unsubstituted aralkyl, which in one embodiment is an aralkyl connected to the molecule via its alkyl moiety (e.g., benzyl). In one embodiment, both R₁ are aralkyl moieties wherein the alkyl portion of the moiety is substituted with two aryl groups and the moiety is connected to the molecule via its alkyl group. For instance, in one embodiment one or both R₁ is a C₇-C₂₄aralkyl wherein the alkyl portion is substituted with two phenyl groups, such as when R₁ is 2,2-diphenylethyl or 2,2-dibenzylethyl. In one embodiment, both R₁ of Formula III is 2,2-diphenylethyl and n is 1, 2 or 5. In one embodiment, each R₁ of Formula III is 2,2-diphenylethyl, n is 1, 2 or 5 and m and p are each 1.

In one embodiment, at least one R₁ is hydrogen. When one R₁ is hydrogen, the other R₁ may be any moiety listed above for R₁, including an aryl group such as benzyl. Any of the compounds of Formula III listed above include compounds where at least one or both of R₂ is hydrogen or a C₁-C₈ substituted or unsubstituted alkyl. In one embodiment, each R₂ is an unsubstituted alkyl such as methyl. In another embodiment, each R₂ is hydrogen. Any of the compounds of Formula III listed above may be compounds where q is 1 and m and p are the same. Accordingly, the polyaminoguanidines of Formula III may be symmetric with reference to the polyaminoguanidine core (e.g. excluding R₁). Alternatively, the compounds of Formula III may be asymmetric, e.g., when q is 0. In one embodiment, m and p are 1. In one embodiment, q is 0. In one embodiment, n is an integer from 1 to 5.

In some embodiments, the compound is a polyaminobiguanide or N-alkylated polyaminobiguanide. An N-alkylated polyaminobiguanide intends a polyaminobiguanide where at least one imine nitrogen of at least one biguanide is alkylated. In one embodiment, the compound is a polyaminobiguanide of the Formula III, or a salt, solvate, or hydrate thereof, where q is 1, and at least one or each R₁ is of the structure:

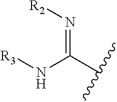

where each R₃ is independently selected from the group consisting of C₁-C₈ alkyl, C₆-C₂₀ aryl, C₆-C₂₀ heteroaryl, C₇-C₂₄ aralkyl, and C₇-C₂₄ heteroaralkyl; and each R₂ is independently hydrogen or a C₁-C₈ alkyl.

In one embodiment, in the polyaminobiguanide compound, at least one or each R₃ is a C₁-C8 alkyl. For instance, when R₃ is a C₁-C₈ alkyl, the alkyl may be substituted with any substituent, including a primary, secondary, tertiary or quaternary amine. Accordingly, in one embodiment, R₃ is a C₁-C₈ alkyl group substituted with an amine such that R₃ may be e.g. alkyl-NH₂ or an alkyl-amine-alkyl moiety such as —(CH₂)_{y}NH(CH₂)_{z}CH₃ where y and z are independently an integer from 1 to 8. In one embodiment, R₃ is —(CH₂)₃NH₂. R₃ may also be a C₄-C₁₅ cycloalkyl or a C₃-C₁₅ branched alkyl. In one embodiment, at least one or each R₃ is a C₆-C₂₀ aryl. In one embodiment, q is l, m and p are 3, and n is 4. In another embodiment, q is l, m and p are 3, and n is 7.

In one embodiment, the compound is a polyaminobiguanide of Formula III where at least one R₃ is a C₇-C₂₄ aralkyl, which in one embodiment is an aralkyl connected to the molecule via its alkyl moiety. In one embodiment, each R₃ is an aralkyl moiety where the alkyl portion of the moiety is substituted with one or two aryl groups and the moiety is connected to the molecule via its alkyl moiety. For instance, in one embodiment at least one or each R₃ is an aralkyl where the alkyl portion is substituted with two phenyl or benzyl groups, such as when R₃ is 2,2-diphenylethyl or 2,2-dibenzylethyl. In one embodiment, each R₃ is 2,2-diphenylethyl and n is 1, 2 or 5. In one embodiment, each R₃ is 2,2-diphenylethyl and n is 1, 2 or 5 and m and p are each 1.

Any of the polyaminobiguanide compounds of Formula (III listed above include compounds where at least one or both of R₂ is hydrogen or a C₁-C₈ alkyl. In one embodiment, each R₂ is an unsubstituted alkyl, such as methyl. In another embodiment, each R₂ is a hydrogen.

Any of the polyaminobiguanide compounds of Formula III listed above include compounds where q is 1 and m and p are the same. Accordingly, the polyaminobiguanides of Formula III may be symmetric with reference to the polyaminobiguanide core. Alternatively, the compounds of Formula III may be asymmetric. In one embodiment, m and p are 1. In one embodiment, q is 0. In one embodiment, n is an integer from 1 to 5. In one embodiment, q, m and p are each 1 and n is 1, 2 or 5.

It is understood and clearly conveyed by this disclosure that each R₁, R₂, R₃, m, n, p and q disclosed in reference to Formula III intends and includes all combinations thereof the same as if each and every combination of R₁, R₂, R₃, m, n, p and q were specifically and individually listed.

Representative compounds of the Formula III include, e.g.:

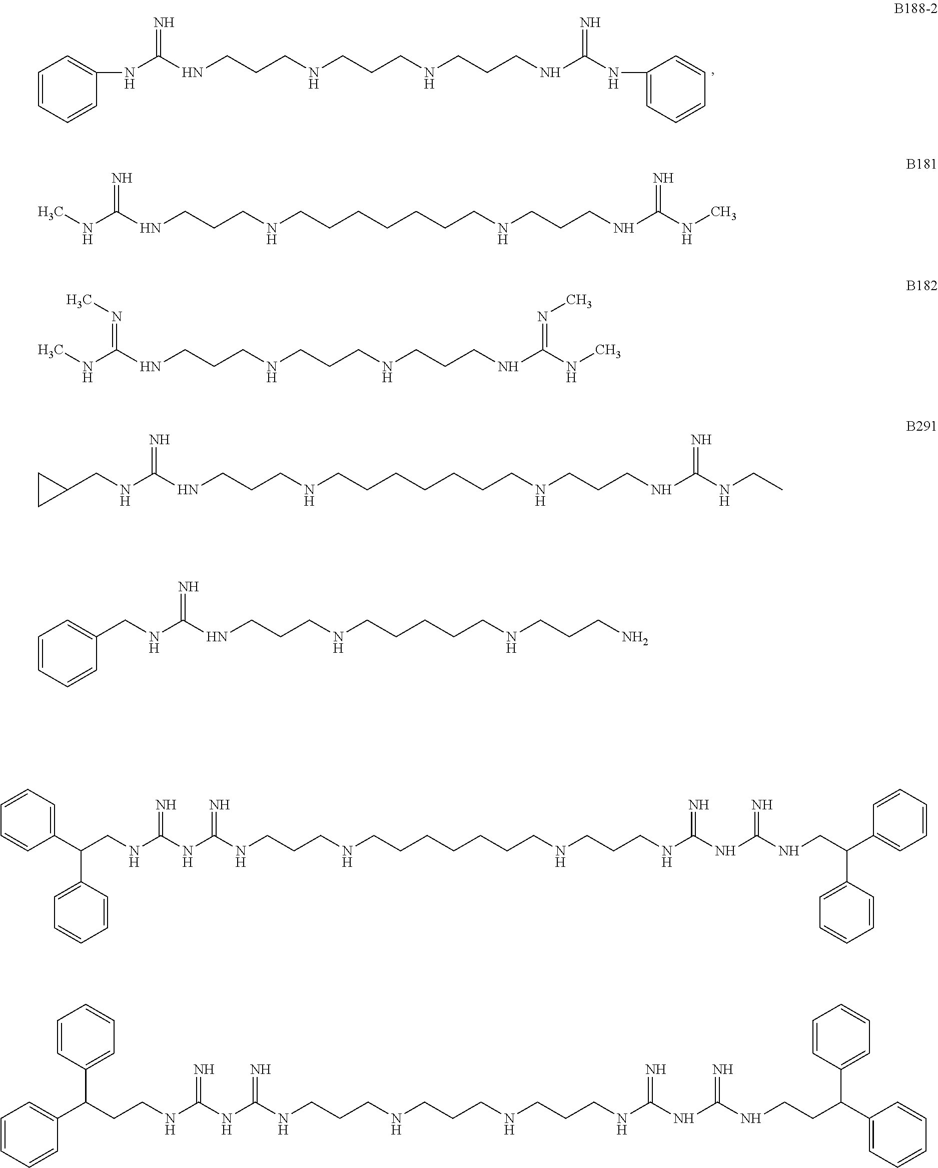

In certain embodiments, the polyamine compound is represented by the structure according to Formula IV:

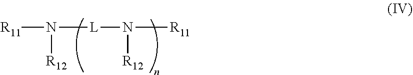

or a salt, solvate or hydrate thereof,
where n is 1, 2 or 3;
each L is independently a linker of from about 2 to 14 carbons in length, for example of about 2, 3, 4, 5, 6, 8, 10, 12 or 14 carbon atoms in length, where the linker backbone atoms may be saturated or unsaturated, usually not more than one, two, three, or four unsaturated atoms will be present in a tether backbone, where each of the backbone atoms may be substituted or unsubstituted (for example with a C₁-C₈ alkyl), where the linker backbone may include a cyclic group (for example, a cyclohex-1,3-diyl group where 3 atoms of the cycle are included in the backbone);
each R₁₂ is independently selected from hydrogen and a C1-C₈ alkyl; and
each R₁₁ is independently selected from hydrogen, C₂-C₈ alkenyl, C₁-C₈ alkyl or C₃-C₈ branched alkyl (e.g., methyl, ethyl, tert-butyl, isopropyl, pentyl, cyclobutyl, cyclopropylmethyl, 3-methylbutyl, 2-ethylbutyl, 5-NH₂-pent-1-yl, propyl-1-ylmethyl(phenyl)phosphinate, dimethylbicyclo[3.1.1]heptyl)ethyl, 2-(decahydronaphthyl)ethyl and the like), C₆-C₂₀ aryl or heteroaryl, C₁-C₂₄ aralkyl or heteroaralkyl (2-phenylbenzyl, 4-phenylbenzyl, 2-benzylbenzyl, 3-benzylbenzyl, 3,3-diphenylpropyl, 3-(benzoimidazolyl)-propyl, 4-isopropylbenzyl, 4-fluorobenzyl, 4-tert-butylbenzyl, 3-imidazolyl-propyl, 2-phenylethyl and the like), —C(═O)—C₁-C₈ alkyl, —C(═O)—C₁-C₈ alkenyl, —C(═O)—C₁-C₈ alkynyl, an amino-substituted cycloalkyl (e.g., a cycloalkyl group substituted with a primary, secondary, tertiary or quaternary amine, such as 5-NH₂-cycloheptyl, 3-NH₂-cyclopentyl and the like) and a C₂-C₈ alkanoyl (e.g., an alkanoyl substituted with a methyl and an alkylazide group).

In certain embodiments, each L is independently selected from: —CHR₁₃—(CH₂)ₘ—, —CHR₁₃—(CH₂)ₙ—CHR₁₃—, —(CH₂)ₘCHR₁₃—, —CH₂-A-CH₂— and —(CH₂)ₚ—

where:
m is an integer from 1 to 5;
A is (CH₂)ₘ, ethane-1,1-diyl or cyclohex-1,3-diyl;
p is an integer from 2 to 14, such as 1, 2, 3, 4 or 5;
n is an integer from 1 to 12; and
R₁₃ is a C₁-C₈ alkyl.

A substituted aralkyl or heteroaralkyl with reference to Formula IV intends and includes alkanoyl moieties substituted with an aryl or heteroaryl group, i.e., —C(═O)-aryl, —C(═O)-aralkyl, —C(═O)-heteroaryl, and —C(═O)-heteroaralkyl. In one embodiment, the alkyl portion of the aralkyl or heteroaralkyl moiety is connected to the molecule via its alkyl moiety. For instance at least one or both of R₁₁ may be an aralkyl moiety such as 2-phenylbenzyl, 4-phenylbenzyl, 3,3, -diphenylpropyl, 2-(2-phenylethyl)benzyl, 2-methyl-3-phenylbenzyl, 2-napthylethyl, 4-(pyrenyl)butyl, 2-(3-methylnapthyl)ethyl, 2-(1,2-dihydroacenaphth-4-yl)ethyl and the like. In another embodiment, at least one or both of R₁₁ may be a heteroaralkyl moiety such as 3-(benzoimidazolyl)propanoyl, 1-(benzoimidazolyl)methanoyl, 2-(benzoimidazolyl)ethanoyl, 2-(benzoimidazolyl)ethyl and the like.

In certain embodiments, the compound of Formula IV comprises at least one moiety selected from the group consisting of t-butyl, isopropyl, 2-ethylbutyl, 1-methylpropyl, 1-methylbutyl, 3-butenyl, isopent-2-enyl, 2-methylpropan-3-olyl, ethylthiyl, phenylthiyl, propynoyl, 1-methyl-1H-pyrrole-2-yl; trifluoromethyl, cyclopropanecarbaldehyde, halo-substituted phenyl, nitro-substituted phenyl, alkyl-substituted phenyl, 2,4,6-trimethylbenzyl, halo-5-substituted phenyl (such as para-(F₃S)-phenyl, azido and 2-methylbutyl.

In certain embodiments, in Formula IV, each R₁₁ is independently selected from hydrogen, n-butyl, ethyl, cyclohexylmethyl, cyclopentylmethyl, cyclopropylmethyl, cycloheptylmethyl, cyclohexyleth-2-yl, and benzyl.

In certain embodiments, the polyamine compound is of the structure of Formula IV, where n is 3, such that the compound has a structure according to Formula V:

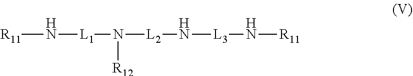

where L₁, L₂ and L₃ are independently selected from —CHR₁₃—(CH₂)ₘ—, —CHR₁₃—(CH₂)ₙ—CHR₁₃—, —(CH₂)ₘ—CHR₁₃—, —CH₂-A-CH₂— and —(CH₂)ₚ—; where m, A, p, n and R₁₃ are as defined above.

In certain embodiments, the polyamine compound is of the structure of Formula V where: L₁ is —CHR₁₃—(CH₂)ₘ—; L₂ is —CHR₁₃—(CH₂)ₙ—CHR₁₃—; and L₃ is —(CH₂)ₘ—CHR₁₃—; where R₁₁, R₁₂, R₁₃, m and n are as defined above.

In certain embodiments, the polyamine compound is of the structure of Formula V where: L₁, L₂ and L₃ are independently —CH₂-A-CH₂—; and R₁₂ is hydrogen; where R₁₁ and A are as defined above. In particular embodiments, at least one of an A and an R₁₁ comprises an alkenyl moiety.

In certain embodiments, the polyamine compound is of the structure of Formula V where: L₁, L₂ and L₃ are independently —(CH₂)ₚ— where p is as defined above; and R₁₂ is hydrogen. In particular embodiments, for L₁ and L₃, p is an integer from 3 to 7, and for L₃ p is an integer from 3 to 14.

In certain embodiments, the polyamine compound is of the structure of Formula V where: L₁, and L₃ are independently —(CH₂)ₚ—; L₂ is —CH₂-A-CH₂—; and R₁₂ is hydrogen; where R₁₂, p and A are as defined above. In particular embodiments, for L₁ and L₃, p is an integer from 2 to 6, and for L₃ A is (CH₂)^{x} where x is an integer from 1 to 5, or cyclohex-1,3-diyl.

In certain embodiments, the polyamine compound is of the structure of Formula IV, where n is 2, such that the compound has a structure according to Formula VI:

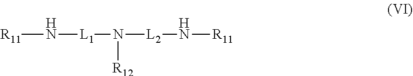

where L₁ and L₂ are independently selected from —CHR₁₃—(CH₂)ₘ—CHR₁₃—(CH₂)ₙ—CHR₁₃—, —(CH₂)ₙ, CHR₁₃—, —CH₂-A-CH₂— and —(CH₂)ₚ—,
where m, A, p, n, and R₁₃ are as defined above.

In certain embodiments, the polyamine compound is of the structure of Formula VI where: L₁ is —(CH₂)ₚ—; and L₂ is —(CH₂)ₘ—CHR₁₃—; where R₁₃, m and p are as defined above. In particular embodiments, for L₁ p is an integer from 3 to 10, and for L₂ n is an integer from 2 to 9.

In certain embodiments, the polyamine compound is of the structure of Formula VI where: L₁ and L₂ are —(CH₂)ₚ—; where p is as defined above. In particular embodiments, p is an integer from 3 to 7.

In certain embodiments, the polyamine compound is of the structure of Formula IV, where n is 1, such that the compound has a structure according to Formula VII:

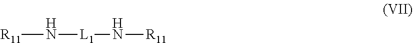

where L₁ is —(CH₂)ₚ— where p is as defined above. In particular embodiments, p is an integer from 2 to 6.

In particular embodiments, in Formula VII, one R₁₁ is an amino-substituted cycloalkyl (e.g. a cycloalkyl group substituted with a primary, secondary, tertiary or quaternary amine) or a C₂-C₈ alkanoyl (which alkanoyl may be substituted with one or more substituents such as a methyl or an alkylazide group); and the other R₁₁ is a C₁-C₈ alkyl or a C₇-C₂₄aralkyl.

Representative compounds of the Formula IIII include, e.g.:

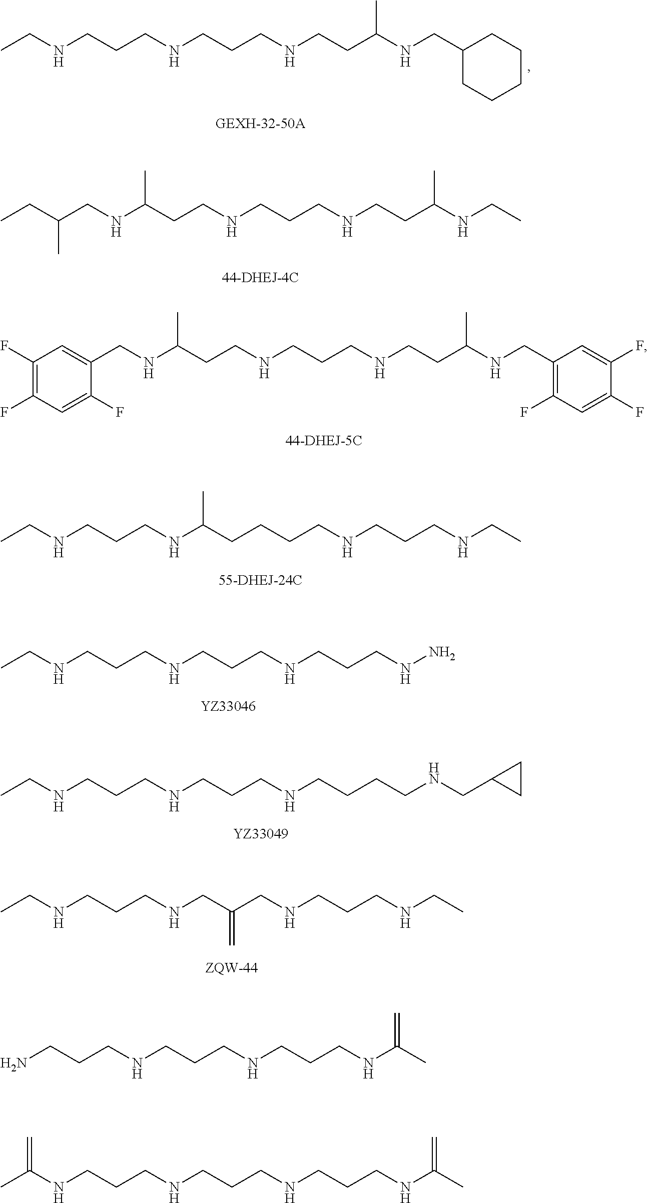

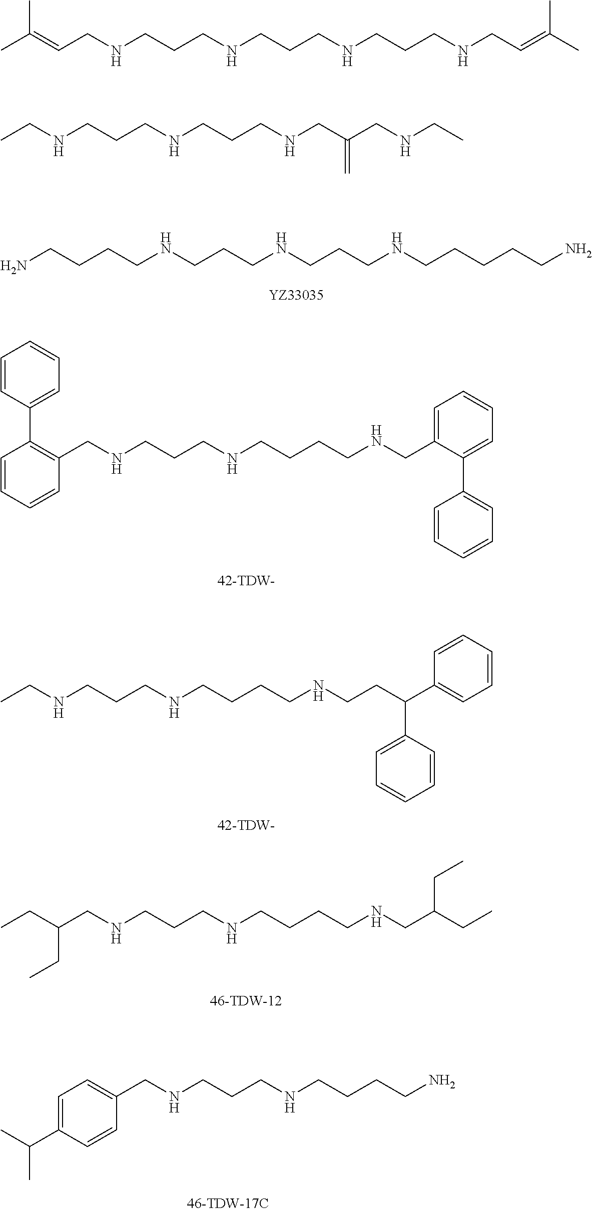

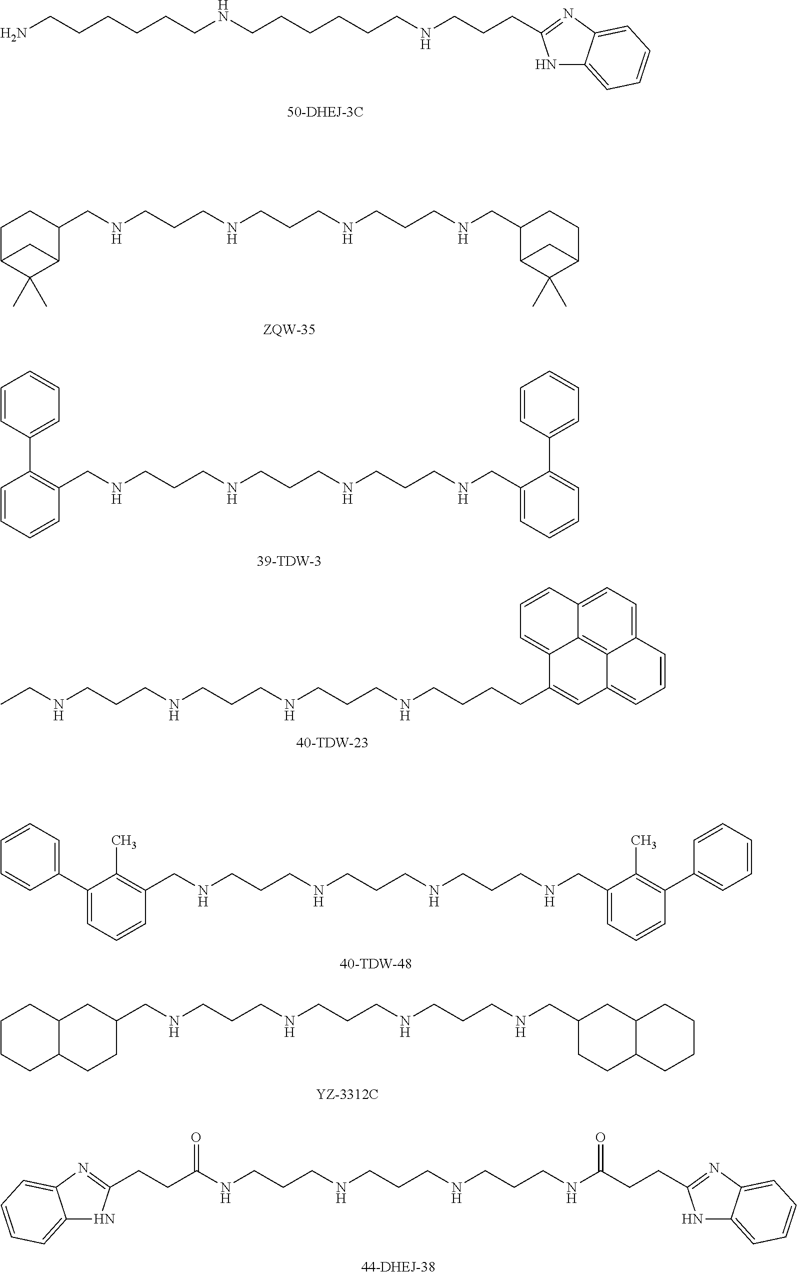

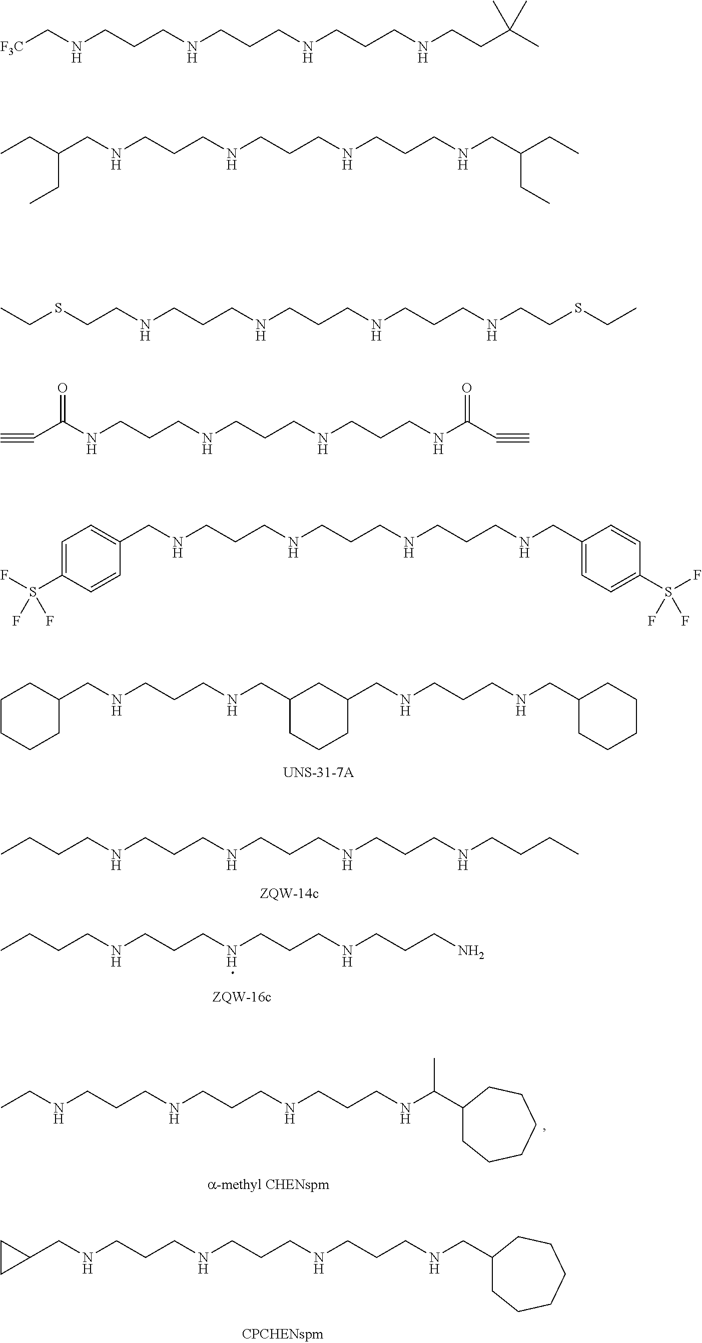

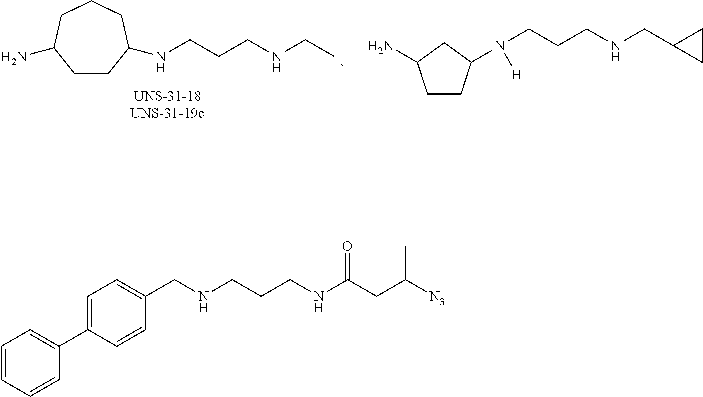 [SMILES: CCNCCCNC1CCCC(N)CC1 ; NC1CCC(NCCCNCC2CC2)C1 ; CC(CC(=O)NCCCNCc1ccc(-c2ccccc2)cc1)N=[N+]=[N-]]

Phenylcyclopropylamine derivatives that are inhibitors of LSD1 include compounds represented by Formula VIII:

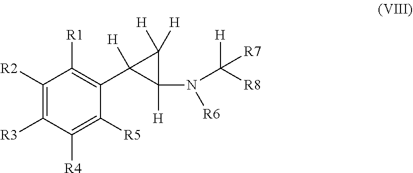

wherein:
each of R1-R5 is independently selected from H, halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
R6 is H or alkyl;
R7 is H, alkyl, or cycloalkyl;
R8 is an -L-heterocyclyl wherein the ring or ring system of the -L-heterocyclyl has from 0 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido; or
R8 is -L-aryl wherein the ring or ring system of the -L-aryl has from 1 to 3 substituents selected from halo, alkyl, alkoxy, cycloalkoxy, haloalkyl, haloalkoxy, -L-aryl, -L-heterocyclyl, -L-carbocyclyl, acylamino, acyloxy, alkylthio, cycloalkylthio, alkynyl, amino, alkylamino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanate, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;
where each L is independently selected from —(CH₂)ₙ—(CH₂)ₙ—, —(CH₂)ₙNH(CH₂)ₙ—, —(CH₂)ₙO(CH₂)ₙ—, and —(CH₂)ₙS(CH₂)ₙ—, and where each n is independently chosen from 0, 1, 2, and 3; or a pharmaceutically acceptable salt thereof.

In some cases, L is a covalent bond. In some cases, R6 and R7 are hydro. In some cases, one of R1-R5 is selected from -L-aryl, -L-heterocyclyl, and -L-carbocyclyl.

In some embodiments of the compound of Formula VIII, the substituent or substituents on the R8 ring or ring system is/are selected from hydroxyl, halo, alkyl, alkoxy, cycloalkoxy, haloalkyl, haloalkoxy, —N(C₁₋₃alkyl)₂, —NH(C₁₋₃ alkyl), —C(═O)NH₂, —C(═O)NH(C₁₋₃ alkyl), —C(═O)N(C₁₋₃ alkyl)₂, —S(═O)₂(C₁₋₃ alkyl), —S(═O)₂NH₂, —S(O)₂NH₂, —S(O)₂N(C₁₋₃ alkyl)₂, —S(═O)₂NH(C₁₋₃alkyl), —CN, —NH₂, and —NO₂.

In certain embodiments, a compound of the invention is of Formula VIII where: each R1-R5 is optionally substituted and independently chosen from —H, halo, alkyl, alkoxy, cycloalkoxy, haloalkyl, haloalkoxy, -L-aryl, -L-heteroaryl, -L-heterocyclyl, -L-carbocyclyl, acylamino, acyloxy, alkylthio, cycloalkylthio, alkynyl, amino, aryl, arylalkyl, arylalkenyl, arylalkynyl, arylalkoxy, aryloxy, arylthio, heteroarylthio, cyano, cyanato, haloaryl, hydroxyl, heteroaryloxy, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido;

R6 is chosen from —H and alkyl;
R7 is chosen from —H, alkyl, and cycloalkyl;
R8 is chosen from —C(═O)NRxRy and —C(═O)Rz;
Rx when present is chosen from —H, alkyl, alkynyl, alkenyl, -L-carbocyclyl, -L-aryl, and -L-heterocyclyl, all of which are optionally substituted (except —H);
Ry when present is chosen from —H, alkyl, alkynyl, alkenyl, -L-carbocyclyl, -L-aryl, and -L-heterocyclyl, all of which are optionally substituted (except —H), where Rx and Ry may be cyclically linked;
Rz when present is chosen from —H, alkoxy, -L-carbocyclyl, -L-heterocyclyl, -L-aryl, wherein the aryl, heterocyclyl, or carbocyclyl are optionally substituted; each L is a linker that links the main scaffold of Formula VIII to a carbocyclyl, heterocyclyl, or aryl group, wherein the hydrocarbon portion of the linker -L- is saturated, partially saturated, or unsaturated, and is independently chosen from a saturated parent group having a formula of —(CH₂)ₙ—(CH₂)ₙ—, —(CH₂)ₙC(═O)(CH₂)—, —(CH₂)ₙC(═O)NH(CH₂)ₙ—, —(CH₂)ₙNHC(O)O(CH₂)ₙ—, —(CH₂)ₙNHC(═O)NH(CH₂)ₙ—, —(CH₂)ₙNHC(═S)S(CH₂)ₙ—, —(CH₂)ₙOC(═O)S(CH₂)ₙ—, —(CH₂)ₙNH(CH₂)ₙ—, —(CH₂)ₙ—O—(CH₂)ₙ—, —(CH₂)ₙS(CH₂)ₙ—, and —(CH₂)ₙNHC(═S)NH(CH₂)ₙ—, where each n is independently chosen from 0, 1, 2, 3, 4, 5, 6, 7, and 8. According to this embodiment, optionally substituted refers to zero or 1 to 4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido. In a more specific aspect of this embodiment, the optional substituents are 1 or 2 optional substituents chosen from halo, alkyl, aryl, and arylalkyl.

In certain embodiments, in Formula VIII, R8 is —CORz, such that the compound is of the following structure:

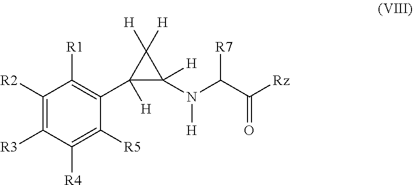

where: R1-R7 are described above; and Rz is -L-heterocyclyl which is optionally substituted with from 1-4 optional substituents independently chosen from acylamino, acyloxy, alkenyl, alkoxy, cycloalkoxy, alkyl, alkylthio, cycloalkylthio, alkynyl, amino, aryl, arylalkyl, arylalkenyl, arylalkynyl, arylalkoxy, aryloxy, arylthio, heteroarylthio, carbocyclyl, cyano, cyanato, halo, haloalkyl, haloaryl, hydroxyl, heteroaryl, heteroaryloxy, heterocyclyl, heteroarylalkoxy, isocyanato, isothiocyanato, nitro, sulfinyl, sulfonyl, sulfonamide, thiocarbonyl, thiocyanato, trihalomethanesulfonamido, O-carbamyl, N-carbamyl, O-thiocarbamyl, N-thiocarbamyl, and C-amido, and wherein said -L- is independently chosen from —(CH₂)ₙ—(CH₂)ₙ—, —(CH₂)ₙNH(CH₂)ₙ—, —(CH₂)ₙ—O—(CH₂)ₙ—, and —(CH₂)ₙS(CH₂)ₙ—, where each n is independently chosen from 0, 1, 2, and 3.

In a specific aspect of this embodiment, each L is independently chosen from —(CH₂)ₙ—(CH₂)ₙ— and —(CH₂)ₙ—O—(CH₂)ₙ where each n is independently chosen from 0, 1, 2, and 3. In a more specific aspect of this embodiment, each L is chosen from a bond, —CH₂—, —CH₂CH₂—, —OCH₂—, —OCH₂CH₂—, —CH₂OCH₂—, —CH₂CH₂CH₂—, —OCH₂CH₂CH₂—, and —CH₂OCH₂CH₂—. In an even more specific aspect, each L is chosen from a bond, —CH₂—, —CH₂CH₂—, OCH₂—, and —CH₂CH₂CH₂—. In yet an even more specific aspect, L is chosen from a bond and —CH₂—.

Exemplary compounds of Formula VIII include:

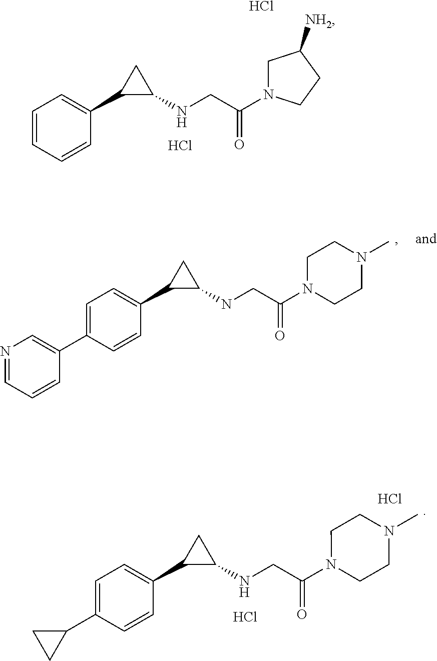

Exemplary compounds of Formula VIII include: N-cyclopropyl-2-{[(trans)-2-phenylcyclopropyl]amino}acetamide; 2-{[(trans)-2-phenylcyclopropyl]amino acetamide; N-cyclopropyl-2-{[(trans)-2-phenylcyclopropyl]amino}propanamide; 2-{[(trans)-2-phenylcyclopropyl]amino}-N-prop-2-ynylacetamide; N-isopropyl-2-{[(trans)-2-phenylcyclopropyl]amino}acetamide; N-(tert-butyl)-2-{[(trans)-2-phenylcyclopropyl]amino}acetamide; N-(2-morpholin-4-yl-2-oxoethyl)-N-[(trans)-2-phenylcyclopropyl]amine; 2-{[(trans)-2-phenylcyclopropyl]amino}propanamide; methyl 2-{[(trans)-2-phenylcyclopropyl]amino}propanoate; N-cyclopropyl-2-{methyl[(trans)-2-phenylcyclopropyl]amino}acetamide; 2-{methyl[(trans)-2-phenylcyclopropyl]amino}acetamide; N-methyl-trans-2-(phenylcyclopropylamino)propanamide; 1-(4-methylpiperazin-1-yl)-2-((trans)-2-phenylcyclopropylamino)ethanone; 1-(4-ethylpiperazin-1-yl)-2-((trans)-2-phenylcyclopropylamino)ethanone; 1-(4-benzylpiperazin-1-yl)-2-((trans)-2-phenylcyclopropylamino)-ethanone; 2-((trans)-2-phenylcyclopropylamino)-1-(4-phenylpiperazin-1-yl)ethanone; 2-((trans)-2-(4-(benzyloxy)phenyl)cyclopropylamino)-1-(4-methylpiperazin-1-yl)ethanone; 2-((trans)-2-(4-(benzyloxy)phenyl)cyclopropylamino)-N-cyclopropylacetamide; 2-((trans)-2-(4-(3-fluorobenzyloxy)phenyl)cyclopropylamino)-1-(4-methylpiperazin-1-yl)ethanone; 2-((trans)-2-(4-(3-chlorobenzyloxy)phenyl)cyclopropylamino)-1-(4-methylpiperazin-1-yl)ethanone; 2-((trans)-2-(biphenyl-4-yl)cyclopropylamino)-1-(4-methylpiperazin-1-yl)ethanone; 1-(4-methylpiperazin-1-yl)-2-((trans)-2-(4-phenethoxyphenyl)cyclopropylamino)ethanone; 2-((trans)-2-(4-(4-fluorobenzyloxy)phenyl)cyclopropylamino)-1-(4-methylpiperazin-1-yl)ethanone; 2-((trans)-2-(4-(biphenyl-4-ylmethoxy)phenyl)cyclopropylamino)-1-(4-methylpiperazin-1-yl)ethanone; (trans)-N-(4-fluorobenzyl)-2-phenylcyclopropanamine; (trans)-N-(4-fluorobenzyl)-2-phenylcyclopropanaminiurn; 4-(((trans)-2-phenylcyclopropylamino)methyl)benzonitrile; (trans)-N-(4-cyanobenzyl)-2-phenylcyclopropanaminium; (trans)-2-phenyl-N-(4-(trifluoromethyl)benzyl)cyclopropanamine; (trans)-2-phenyl-N-(4-(trifluoromethyl)benzyl)cyclopropanaminium; (trans)-2-phenyl-N-(pyridin-2-ylmethyl)cyclopropanamine; (trans)-2-phenyl-N-(pyridin-3-ylmethyl)cyclopropanamine; (trans)-2-phenyl-N-(pyridin-4-ylmethyl)cyclopropanamine; (trans)-N-((6-methylpyridin-2-yl)methyl)-2-phenylcyclopropanamine; (trans)-2-phenyl-N-(thiazol-2-ylmethyl)cyclopropanamine; (trans)-2-phenyl-N-(thiophen-2-ylmethyl)cyclopropanamine; (trans)-N-((3-bromothiophen-2-yl)methyl)-2-phenylcyclopropanamine; (trans)-N-((4-bromothiophen-2-yl)methyl)-2-phenylcyclopropanamine; (trans)-N-(3,4-dichlorobenzyl)-2-phenylcyclopropanamine; (trans)-N-(3-fluorobenzyl)-2-phenylcyclopropanaminium; (trans)-N-(2-fluorobenzyl)-2-phenylcyclopropanamine; (trans)-2-phenyl-N-(quinolin-4-ylmethyl)cyclopropanaraine; (trans)-N-(3-methoxybenzyl)-2-phenylcyclopropanamine; (trans)-2-phenyl-N-((6-(trifluoromethyl)pyridin-3-yl)methyl)cyclopropanamine; (trans)-N-((6-chloropyridin-3-yl)methyl)-2-phenylcyclopropanamine; (trans)-N-((4-methylpyridin-2-yl)methyl)-2-phenylcyclopropanamine; (trans)-N-((6-methoxypyridin-2-yl)methyl)-2-phenylcyclopropanamine; 2-(((trans)-2-phenylcyclopropylamino)methyl)pyridin-3-ol; (trans)-N-((6-bromopyridin-2-yl)methyl)-2-phenylcyclopropanamine; 4-(((trans)-2-(4(benzyloxy)phenyl)cyclopropylamino)methyl)benzonitrile; (trans)-N-(4-(benzyloxy)benzyl)-2-phenylcyclopropanamine; (trans)-N-benzyl-2-(4-(benzyloxy)phenyl)cyclopropanamine; (trans)-2-(4-(benzyloxy)phenyl)-N-(4-methoxybenzyl)cyclopropanamine; (trans)-2-(4-(benzyloxy)phenyl)-N-(4-fluorobenzyl)cyclopropanamine-; (trans)-2-phenyl-N-(quinolin-2-ylmethyl)cyclopropanamine; (trans)-2-phenyl-N-((5-(trifluoromethyl)pyridin-2-yl)methyl)cyclopropanamine; (trans)-N-((3-fluoropyridin-2-yl)methyl)-2-phenylcyclopropanamine; (trans)-2-phenyl-N-(quinolin-3-ylmethyl)cyclopropanamine; (trans)-N-((6-methoxypyridin-3-yl)methyl)-2-phenylcyclopropanamine; (trans)-N-((5-methoxypyridin-3-yl)methyl)-2-phenylcyclopropanamine-; (trans)-N-((2-methoxypyridin-3-yl)methyl)-2-phenylcyclopropanamine; (trans)-N-((3H-indol-3-yl)methyl)-2-phenylcyclopropanamine; 3-(((trans)-2-phenylcyclopropylamino)methyl)benzonitrile; (trans)-N-(2-methoxybenzyl)-2-phenylcyclopropanamine; 3-(((trans)-2-phenylcyclopropylamino)methyl)pyridin-2-amine; (trans)-N-((2-chloropyridin-3-yl)methyl)-2-phenylcyclopropanamine; (trans)-N-(3,4-dimethoxybenzyl)-2-phenylcyclopropanamine; (trans)-N-((2,3-dihydrobenzofuran-5-yl)methyl)-2-phenylcyclopropanamine; (trans)-N-(benzo[d][1,3]dioxol-5-ylmethyl)-2-phenylcyclopropanamine; (trans)-N-((2,3-dihydrobenzo[b][1,4]dioxin-6-yl)methyl)-2-phenyl-cyclopropanamine; (trans)-N-(2,6-difluoro-4-methoxybenzyl)-2-phenylcyclopropanamine; (trans)-2-phenyl-N-(4-(trifluoromethoxy)benzyl)cyclopropanamine; (trans)-N-(5-fluoro-2-methoxybenzyl)-2-phenylcyclopropanamine; (trans)-N-(2-fluoro-4-methoxybenzyl)-2-phenylcyclopropanamine; (trans)-N-((4-methoxynaphthalen-1-yl)methyl)-2-phenylcyclopropanamine; (trans)-N-(2-fluoro-6-methoxybenzyl)-2-phenylcyclopropanamine; (trans)-N-((2-methoxynaphthalen-1-yl)methyl)-2-phenylcyclopropanamine; (trans)-N-((4,7-dimethoxynaphthalen-1-yl)methyl)-2-phenylcyclopropanamine; (trans)-N-(4-methoxy-3-methylbenzyl)-2-phenylcyclopropanamine; (trans)-N-(3-chloro-4-methoxybenzyl)-2-phenylcyclopropanamine; (trans)-N-(3-fluoro-4-methoxybenzyl)-2-phenylcyclopropanamine; (trans)-N-(4-methoxy-2-methylbenzyl)-2-phenylcyclopropanamine; (trans)-N-((3,4-dihydro-2H-benzo[b][1,4]dioxepin-6-yl)methyl)-2-phenylcyclopropanamine; (trans)-N-((3,4-dihydro-2H-benzo[b][1,4]dioxepin-7-yl)methyl)-2-phenylcyclopropanamine; (trans)-N-((2,2-dimethylchroman-6-yl)methyl)-2-phenylcyclopropanamine; (trans)-N-(4-methoxy-2,3-dimethylbenzyl)-2-phenylcyclopropanamine; (trans)-N-(4-methoxy-2,5-dimethylbenzyl)-2-phenylcyclopropanamine; (trans)-N-(2-fluoro-4,5-dimethoxybenzyl)-2-phenylcyclopropanamine; (trans)-N-(3-chloro-4,5-dimethoxybenzyl)-2-phenylcyclopropanamine; (trans)-N-(2-chloro-3,4-dimethoxybenzyl)-2-phenylcyclopropanamine; (trans)-N-(2,4-dimethoxy-6-methylbenzyl)-2-phenylcyclopropanamine; (trans)-N-(2,5-dimethoxybenzyl)-2-phenylcyclopropanamine; (trans)-N-(2,3-dimethoxybenzyl)-2-phenylcyclopropanamine; (trans)-N-(2-chloro-3-methoxybenzyl)-2-phenylcyclopropanamine; (trans)-N-((1H-indol-5-yl)methyl)-2-phenylcyclopropanamine; (trans)-2-(4-(benzyloxy)phenyl)-N-(pyridin-2-ylmethyl)cyclopropanamine; (trans)-2-(4-(benzyloxy)phenyl)-N-(2-methoxybenzyl)cyclopropanamine; (trans)-N-(1-(4-methoxyphenyl)ethyl)-2-phenylcyclopropanaraine; (trans)-N-(1-(3,4-dimethoxyphenyl)ethyl)-2-phenylcyclopropanamine; (trans)-N-(1-(2,3-dihydrobenzo[b][1,4]dioxin-6-yl)ethyl)-2-phenylcyclopropanamine; (trans)-N-(1-(5-fluoro-2-methoxyphenyl)ethyl)-2-phenylcyclopropanamine; (trans)-N-(1-(3,4-dimethoxyphenyl)propan-2-yl)-2-phenylcyclopropanamine; (trans)-N-((3-methyl-1,2,4-oxadiazol-5-yl)methyl)-2-phenylcyclopropanamine; and pharmaceutically acceptable salts thereof.

Alternative small molecule LSD1 inhibitor compounds may be selected from selective LSD1 and LSD1/MAOB dual inhibitors disclosed, for example, in WO2010/043721 (PCT/EP2009/063685), WO2010/084160 (PCT/EP2010/050697), PCT/EP2010/055131; PCT/EP2010/055103; and EP application number EP10171345 all of which are explicitly incorporated herein by reference in their entireties to the extent they are not inconsistent with the instant disclosure. Representative compounds of this type include phenylcyclopropylamine derivatives or homologs, illustrative examples of which include phenylcyclopropylamine with one or two substitutions on the amine group; phenylcyclopropylamine with zero, one or two substitutions on the amine group and one, two, three, four, or five substitution on the phenyl group; phenylcyclopropylamine with one, two, three, four, or five substitution on the phenyl group; phenylcyclopropylamine with zero, one or two substitutions on the amine group wherein the phenyl group of PCPA is substituted with (exchanged for) another ring system chosen from aryl or heterocyclyl to give an aryl- or heteroaryl-cyclopropylamine having zero, one or two substituents on the amine group; phenylcyclopropylamine wherein the phenyl group of PCPA is substituted with (exchanged for) another ring system chosen from aryl or heterocyclyl to give an aryl- or heterocyclyl-cyclopropylamine wherein said aryl- or heterocyclyl-cyclopropylamine on said aryl or heterocyclyl moiety has zero, one or two substitutions on the amine group and one, two, three, four, or five substitution on the phenyl group; phenylcyclopropylamine with one, two, three, four, or five substitution on the phenyl group; or any of the above described phenylcyclopropylamine analogs or derivatives wherein the cyclopropyl has one, two, three or four additional substituents. Suitably, the heterocyclyl group described above in this paragraph in a heteroaryl.

Non-limiting embodiments of phenylcyclopropylamine derivatives or analogs include “cyclopropylamine amide” derivatives and “cyclopropylamine” derivatives. Specific examples of “cyclopropylamine acetamide” derivatives include, but are not limited to: N-cyclopropyl-2-{[(trans)-2-phenylcyclopropyl]amino}acetamide; 2-{[(trans)-2-phenylcyclopropyl]amino}acetamide; N-cyclopropyl-2-{[(trans)-2-phenylcyclopropyl]amino}propanamide; 2-{[(trans)-2-phenylcyclopropyl]amino}-N-prop-2-ynylacetamide; N-isopropyl-2-{[(trans)-2-phenylcyclopropyl]amino}acetamide; N-(tert-butyl)-2-{[(trans)-2-phenylcyclopropyl]amino}acetamide; N-(2-morpholin-4-yl-2-oxoethyl)-N-[(trans)-2-phenylcyclopropyl]amine; 2-{[(trans)-2-phenylcyclopropyl]amino}propanamide; methyl 2-{[(trans)-2-phenylcyclopropyl]amino}propanoate; 1-(4-methylpiperazin-1-yl)-2-((trans)-2-phenylcyclopropylamino)ethanone; 1-(4-ethylpiperazin-1-yl)-2-((trans)-2-phenylcyclopropylamino)ethanone; 1-(4-benzylpiperazin-1-yl)-2-((trans)-2-phenylcyclopropylamino)ethanone; 2-((trans)-2-phenylcyclopropylamino)-1-(4-phenylpiperazin-1-yl)ethanone; 2-((trans)-2-(4-(benzyloxy)phenyl)cyclopropylamino)-1-(4-methylpiperazin-1-yl)ethanone; 2-((trans)-2-(1,1′-biphenyl-4-yl)cyclopropylamino)-1-(4-methylpiperazin-1-yl)ethanone; 2-((trans)-2-(4-(benzyloxy)phenyl)cyclopropylamino)-N-cyclopropylacetamide; 2-((trans)-2-(4-(3-fluorobenzyloxy)phenyl)cyclopropylamino)-1-(4-methylpiperazin-1-yl)ethanone; 2-((trans)-2-(4-(4-fluorobenzyloxy)phenyl)cyclopropylamino)-1-(4-methylpiperazin-1-yl)ethanone; 2-((trans)-2-(4-(3-chlorobenzyloxy)phenyl)cyclopropylamino)-1-(4-methylpiperazin-1-yl)ethanone; 1-(4-methylpiperazin-1-yl)-2-((trans)-2-(4-phenethoxyphenyl)cyclopropylamino)ethanone; 2-((trans)-2-(biphenyl-4-yl)cyclopropylamino)-1-(4-methylpiperazin-1-yl)ethanone; N-cyclopropyl-2-{[(trans)-2-phenylcyclopropyl]amino}acetamide; N-methyl-trans-2-(phenylcyclopropylamino)propanamide; 2-{methyl[(trans)-2-phenylcyclopropyl]amino}acetamide; N-[2-(4-methylpiperazin-1-yl)ethyl]-N-[(trans)-2-phenylcyclopropyl]amine; N-cyclopropyl-N′-[(trans)-2-phenylcyclopropyl]ethane-1,2-diamine; N,N-dimethyl-N′-(2-{[(trans)-2-phenylcyclopropyl]amino}ethyl)ethane-1,2-diamine; (3R)-1-(2-{[(trans)-2-phenylcyclopropyl]amino}ethyl)pyrrolidin-3-amine; (3S)—N,N-dimethyl-1-(2-{[(trans)-2-phenylcyclopropyl]amino}ethyl)pyrrolidin-3-amine; (3R)—N,N-dimethyl-1-(2-{[(trans)-2-phenylcyclopropyl]amino}ethyl)pyrrolidin-3-amine; N-[(trans)-2-phenylcyclopropyl]-N-(2-piperazin-1-ylethyl)amine; N,N-diethyl-N′-[(trans)-2-phenylcyclopropyl]ethane-1,2-diamine; N-[(trans)-2-phenylcyclopropyl]-N-(2-piperidin-1-ylethyl)amine; (trans)-2-(4-(benzyloxy)phenyl)-N-(2-(4-methylpiperazin-1-yl)ethyl)cyclopropanamine; (trans)-N-(2-(4-methylpiperazin-1-yl)ethyl)-2-(3′-(trifluoromethyl)biphenyl-4-yl)cyclopropanamine; (trans)-2-(3′-chlorobiphenyl-4-yl)-N-(2-(4-methylpiperazin-1-yl)ethyl)cyclopropanamine; (R)-1-(2-((trans)-2-(3′-(trifluoromethyl)biphenyl-4-yl)cyclopropylamino)ethyl)pyrrolidin-3-amine; and N′-cyclopropyl-N²-((trans)-2-(3′-(trifluoromethyl)biphenyl-4-yl)cyclopropyl)ethane-1,2-diamine.

Specific examples of “cyclopropylamine” derivatives, include, but are not limited to: N-4-fluorobenzyl-N-{(trans)-2-[4-(benzyloxy)phenyl]cyclopropyl}amine, N-4-methoxybenzyl-N-{(trans)-2-[4-(benzyloxy)phenyl]cyclopropyl}amine, N-benzyl-N-{(trans)-2-[4-(benzyloxy)phenyl]cyclopropyl}amine, N-[(trans)-2-phenylcyclopropyl]amino-methyl)pyridin-3-ol, N-[(trans)-2-phenylcyclopropyl]-N-(3-methylpyridin-2-ylmethyl)amine, N-[(trans)-2-phenylcyclopropyl]-N-(4-chloropyridin-3-ylmethyl)amine, N-[(trans)-2-phenylcyclopropyl]-N-(4-trifluoromethylpyridin-3-yl-methyl)amine, N-(3-methoxybenzyl)-N-[(trans)-2-phenylcyclopropyl]amine, N-[(trans)-2-phenylcyclopropyl]-N-(quinolin-4-ylmethyl)amine, N-(2-fluorobenzyl)-N-[(trans)-2-phenylcyclopropyl]amine, N-(3-fluorobenzyl)-N-[(trans)-2-phenylcyclopropyl]amine, N-[(trans)-2-phenylcyclopropyl]-N-(3,4-dichloro-1-phenylmethyl)amine, N-[(trans)-2-phenylcyclopropyl]-N-(5-bromo-thiophen-2-ylmethyl)amine, N-[(trans)-2-phenylcyclopropyl]-N-(3-bromo-thiophen-2-ylmethyl)- amine, N-[(trans)-2-phenylcyclopropyl]-N-(thiophen-2-ylmethyl)amine, N-[(trans)-2-phenylcyclopropyl]-N-(1,3-thiazol-2-ylmethyl)amine, N-[(trans)-2-phenylcyclopropyl]-N-(3-methyl-pyridin-2-ylmethyl)amine, N-[(trans)-2-phenylcyclopropyl]-N-(pyridin-4-ylmethyl)amine, N-[(trans)-2-phenylcyclopropyl]-N-(pyridin-3-ylmethyl)amine, N-[(trans)-2-phenylcyclopropyl]-N-(pyridin-2-ylmethyl)amine, [(trans)-2-phenylcyclopropyl]-N-[4-(trifluoromethyl)benzyl]amine, ({[(trans)-2-phenylcyclopropyl]amino}methyl)benzonitrile, N-(4-fluorobenzyl)-N-[(trans)-2-phenylcyclopropyl]amine, N-[(trans)-2-phenylcyclopropyl]-N-(3-bromo-pyridin-2-ylmethyl)amine, N-4-cyanobenzyl-N-{(trans)-2-[4-(benzyloxy)phenyl]cyclopropyl}amine, N-4-[(benzyloxy)-benzyl]-N-[(trans)-2-(4-phenyl)cyclopropyl]amine; 2-((trans)-2-(4-(4-cyanobenzyloxy)phenyl)cyclopropylamino)acetamide, 2-((trans)-2-(4-(3-cyanobenzyloxy)phenyl)cyclopropylamino)acetamide, 2-((trans)-2-(4-(benzyloxy)phenyl)cyclopropylamino)acetamide, 2-((trans)-2-(4-(4-fluorobenzyloxy)phenyl)cyclopropylamino)acetamide, 2-((trans)-2-(4-(3-fluorobenzyloxy)phenyl)cyclopropylamino)acetamide, 2-((trans)-2-(4-(3-chlorobenzyloxy)phenyl)cyclopropylamino)acetamide, 2-((trans)-2-(4-(4-chlorobenzyloxy)phenyl)cyclopropylamino)acetamide, 2-((trans)-2-(4-(3-bromobenzyloxy)phenyl)cyclopropylamino)acetamide, 2-((trans)-2-(4-(3,5-difluorobenzyloxy)phenyl)cyclopropylamino)acetamide, 2-((trans)-2-(4-phenethoxyphenyl)cyclopropylamino)acetamide, 2-((trans)-2-(3′-(trifluoromethyl)biphenyl-4-yl)cyclopropylamino)acetamide, and 2-((trans)-2-(3′-chlorobiphenyl-4-yl)cyclopropylamino)acetamide.

Other examples of LSD1 inhibitors are, e.g., phenelzine or pargyline (propargylamine) or a derivative or analog thereof. Derivatives and analogs of phenelzine and pargyline (propargylamine) include, but are not limited to, compounds where the phenyl group of the parent compound is replaced with a heteroaryl or optionally substituted cyclic group or the phenyl group of the parent compound is optionally substituted with a cyclic group. In one aspect, the phenelzine or pargyline derivative or analog thereof has selective LSD1 or dual LSD1/MAOB inhibitory activity as described herein. In some embodiments, the phenelzine derivative or analog has one, two, three, four or five substituents on the phenyl group. In one aspect, the phenelzine derivative or analog has the phenyl group substituted with (exchanged for) an aryl or heterocyclyl group wherein said aryl or heterocyclyl group has zero, one, two, three, four or five substituents. In one aspect, the pargyline derivative or analog has one, two, three, four or five substituents on the phenyl group. In one aspect, the pargyline derivative or analog has the phenyl group substituted with (exchanged for) an aryl or heterocyclyl group wherein said aryl or heterocyclyl group has zero, one, two, three, four or five substituents. Methods of preparing such compounds are known to the skilled artisan.

The present invention also contemplates tranylcypromine derivatives as described for example by Binda et al. (2010, J. Am. Chem. Soc., 132: 6827-6833, which is hereby incorporated by reference herein in its entirety) as inhibitors of LSD1 catalytic function. Non-limiting example of such compounds include:

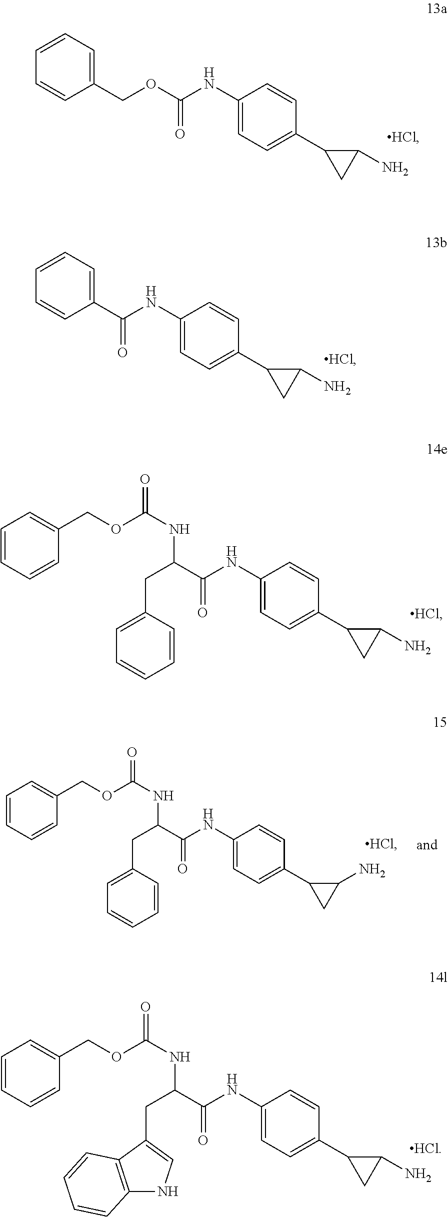

Alternatively, LSD1 inhibitor compounds may be selected from tranylcypromine analogs described by Benelkebir et al. (2011, Bioorg. Med. Chem. 19(12): 3709-16, which is hereby incorporated by reference herein in its entirety). Representative analogs of this type, including o-, m- and p-bromo analogues include: (1R,2S)-2-(4-bromophenyl)cyclopropanamine hydrochloride (Compound 4c), (1R,2S)-2-(3-bromophenyl)cyclopropanamine hydrochloride (Compound 4d), (1R,2S)-2-(2-bromophenyl)cyclopropanamine hydrochloride (Compound 4e), (1R,2S)-2-(biphenyl-4-yl)cyclopropanamine hydrochloride (Compound 4f).

Reference also may be made to peptide scaffold compounds disclosed by Culhane et al. (2010, J. Am. Chem. Soc., 132: 3164-3176, which is hereby incorporated by reference herein in its entirety), which include chlorovinyl, endo-cyclopropylamine, and hydrazine functionalities. Non-limiting compounds disclosed by Culhane et al. include propargyl-Lys-4, N-methylpropargyl-Lys-4 H3-21, cis-3-chloroallyl-Lys-4H3-21, trans-3-chloroallyl-Lys-4H3-21, exo-cyclopropyl-Lys-4H3-21, endo-cyclopropyl-Lys-4H3-21, endo-dimethylcyclopropyl-Lys-4, hydrazino-Lys-4H3-21 and hydrazino-Lys-4H3-21.

Alternative cyclopropylamine compounds that are useful for inhibiting LSD1 include those disclosed by Fyfe et al. in U.S. Publication No. 2013/0197013, which is incorporated herein by reference in its entirety. Illustrative cyclopropylamine inhibitors of LSD1, which are disclosed as being selective for inhibiting LSD1, include compounds according to Formula IX:

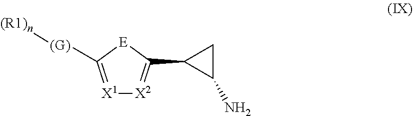

wherein:
E is —N(R3)-, —O—, or —S—, or is —X³═X⁴—;
X¹ and X² are independently C(R2) or N;
X³ and X⁴, when present, are independently C(R2) or N;
(G) is a cyclyl group (as shown in Formula IX, the cyclyl group (G) has n substituents (R1));
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from —H, alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 independently chosen optional substituents or two (R2) groups can be taken together to form a heterocyclyl or aryl group having 1, 2, or 3 independently chosen optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group;
each L1 is independently alkylene or heteroalkylene; and
n is 0, 1, 2, 3, 4 or 5,
or an enantiomer, a diastereomer, or a mixture thereof, or a pharmaceutically acceptable salt or solvate thereof.

In some embodiments, compounds of Formula IX are represented by Formula X:

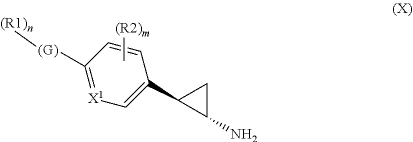

wherein:
X¹ is CH or N; (G) is a cyclyl group;
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
each L1 is independently alkylene or heteroalkylene;
m is 0, 1, 2 or 3; and n is 0, 1, 2, 3, 4 or 5, provided that n and m are chosen independently such that n+m is greater than zero when X¹ is —CH— and (G) is an aryl;
or an enantiomer, a diastereomer, or a mixture thereof, or a pharmaceutically acceptable salt or solvate thereof.

In other embodiments, compounds of Formula IX are represented by Formula XI:

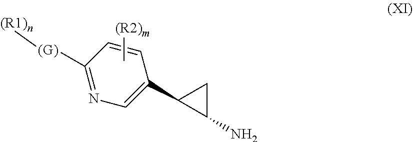

wherein:
(G) is a cyclyl group;
each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 0, 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
each L1 is independently alkylene or heteroalkylene; m is 0, 1, 2 or 3; and
n is 0, 1, 2, 3, 4 or 5;
or an enantiomer, a diastereomer, or a mixture thereof, or a pharmaceutically acceptable salt or solvate thereof.

In still other embodiments, compounds of formula IX are represented by Formula XII:

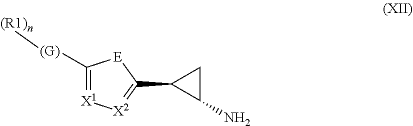

wherein:
E is —N(R3)-, —O—, or —S—, or is —X³═X⁴—;
X¹, X², X³ and X⁴ are independently C(R2) or N, provided that at least one of X¹, X², X³ and X⁴ is N when E is —X³═X⁴—;
(G) is a cyclyl group; each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate;
R3 is —H or a (C₁-C₆)alkyl group; each L1 is alkylene or heteroalkylene; and n is 0, 1, 2, 3, 4 or 5;
or an enantiomer, a diastereomer, or a mixture thereof, or a pharmaceutically acceptable salt or solvate thereof.

In still other embodiments, compounds of Formula IX are represented by Formula XIII:

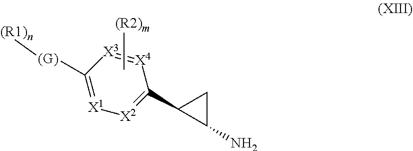

wherein:
X¹, X², X³ and X⁴ are independently CH or N, provided that at least one of X¹, X², X³ and X⁴ is N;
(G) is a cyclyl group; each (R1) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl;
each (R2) is independently chosen from alkyl, alkenyl, alkynyl, cyclyl, -L1-cyclyl, -L1-amino, -L1-hydroxyl, amino, amido, nitro, halo, haloalkyl, haloalkoxy, cyano, sulfinyl, sulfonyl, sulfonamide, hydroxyl, alkoxy, urea, carbamate, acyl, or carboxyl, wherein each (R2) group has 1, 2, or 3 optional substituents, wherein said optional substituents are independently chosen from alkyl, alkanoyl, heteroalkyl, heterocyclyl, haloalkyl, cycloalkyl, carbocyclyl, arylalkoxy, heterocyclylalkoxy, aryl, aryloxy, heterocyclyloxy, alkoxy, haloalkoxy, oxo, acyloxy, carbonyl, carboxyl, carboxamido, cyano, halogen, hydroxyl, amino, aminoalkyl, amidoalkyl, amido, nitro, thiol, alkylthio, arylthio, sulfonamide, sulfinyl, sulfonyl, urea, or carbamate; each L1 is alkylene or heteroalkylene;
m is 0, 1, 2 or 3; and n is 0, 1, 2, 3, 4 or 5;
or an enantiomer, a diastereomer, or a mixture thereof, or a pharmaceutically acceptable salt or solvate thereof.

Representative compounds according to Formula IX are suitably selected from: (trans)-2-(3′-(trifluoromethyl)biphenyl-4-yl)cyclopropanamine; (trans)-2-(terphenyl-4-yl)cyclopropanamine; 4′-((trans)-2-aminocyclopropyl)biphenyl-4-ol; 4′-((trans)-2-aminocyclopropyl)biphenyl-3-ol; (trans)-2-(6-(3-(trifluoromethyl)phenyl)pyridin-3-yl)cyclopropanamine; (trans)-2-(6-(3,5-dichlorophenyl)pyridin-3-yl)cyclopropanamine; (trans)-2-(6-(4-chlorophenyl)pyridin-3-yl)cyclopropanamine; (trans)-2-(6-(3-chlorophenyl)pyridin-3-yl)cyclopropanamine; (trans)-2-(6-(4-(trifluoromethyl)phenyl)pyridin-3-yl)cyclopropanamine; (trans)-2-(6-(4-methoxyphenyl)pyridin-3-yl)cyclopropanamine; (trans)-2-(6-(3-methoxyphenyl)pyridin-3-yl)cyclopropanamine; 4-(5-((trans)-2-aminocyclopropyl)pyridin-2-yl)benzonitrile; 3-(5-((trans)-2-aminocyclopropyl)pyridin-2-yl)benzonitrile; (trans)-2-(6-p-tolylpyridin-3-yl)cyclopropanamine; (trans)-2-(6-m-tolylpyridin-3-yl)cyclopropanamine; 4-(5-((trans)-2-aminocyclopropyl)pyridin-2-yl)phenol; 3-(5-((trans)-2-aminocyclopropyl)pyridin-2-yl)phenol; 4-(5-((trans)-2-aminocyclopropyl)pyridin-2-yl)benzamide; 3-(5-((trans)-2-aminocyclopropyl)pyridin-2-yl)benzamide; 2-(5-((trans)-2-aminocyclopropyl)pyridin-2-yl)phenol; 3-(5-((trans)-2-aminocyclopropyl)pyridin-2-yl)phenol; (trans)-2-(6-(3-methoxy-4-methylphenyl)pyridin-3-yl)cyclopropanamine; 5-(5-((trans)-2-aminocyclopropyl)pyridin-2-yl)-2-fluorophenol; 3-(5-((trans)-2-aminocyclopropyl)pyridin-2-yl)-5-fluorophenol; 3-(5-((trans)-2-aminocyclopropyl)pyridin-2-yl)-4-fluorophenol; 3-(5-((trans)-2-aminocyclopropyl)pyridin-2-yl)-2-fluorophenol; 3-(5-((trans)-2-aminocyclopropyl)pyridin-2-yl)-2,4-difluorophenol; 3-(5-((trans)-2-aminocyclopropyl)pyridin-2-yl)-2,4,6-trifluorophenol; 3-(5-((trans)-2-aminocyclopropyl)pyridin-2-yl)-5-chlorophenol; (trans)-2-(6-(2-fluoro-3-(trifluoromethyl)phenyl)pyridin-3-yl)cyclopropanamine; (trans)-2-(6-(5-chlorothiophen-2-yl)pyridin-3-yl)cyclopropanamine; (trans)-2-(6-(5-methylthiophen-2-yl)pyridin-3-yl)cyclopropanamine; (trans)-2-(6-(1H-indol-6-yl)pyridin-3-yl)cyclopropanamine; (trans)-2-(6-(benzo[b]thiophen-5-yl)pyridin-3-yl)cyclopropanamine; 3-(5-((trans)-2-aminocyclopropyl)-3-methylpyridin-2-yl)phenol; (trans)-2-(6-(3-chlorophenyl)-5-methylpyridin-3-yl)cyclopropanamine; (trans)-2-(5-methyl-6-(3-(trifluoromethyl)phenyl)pyridin-3-yl)cyclopropanamine; (trans)-2-(6-(4-fluoro-3-methoxyphenyl)pyridin-3-yl)cyclopropanamine, (trans)-2-(6-(3-fluoro-5-methoxyphenyl)pyridin-3-yl)cyclopropanamine; (trans)-2-(6-(2-fluoro-5-methoxyphenyl)pyridin-3-yl)cyclopropanamine, (trans)-2-(6-(2-fluoro-3-methoxyphenyl)pyridin-3-yl)cyclopropanamine; (trans)-2-(6-(3-chloro-5-methoxyphenyl)pyridin-3-yl)cyclopropanamine; (trans)-2-(6-(2-chloro-5-methoxyphenyl)pyridin-3-yl)cyclopropanamine; (trans)-2-(6-(3-methoxy-5-(trifluoromethyl)phenyl)pyridin-3-yl)cyclopropanamine; 3-(5-((trans)-2-aminocyclopropyl)pyridin-2-yl)-5-methoxybenzonitrile; 5-(5-((trans)-2-aminocyclopropyl)pyridin-2-yl)-2-methylphenol; 3-(5-((trans)-2-aminocyclopropyl)pyridin-2-yl)-4-chlorophenol; 3-(5-((trans)-2-aminocyclopropyl)pyridin-2-yl)-5-(trifluoromethyl)phenol; (trans)-2-(6-(2-fluoro-5-(trifluoromethyl)phenyl)pyridin-3-yl)cyclopropanamine; (trans)-2-(6-(2-chloro-5-(trifluoromethyl)phenyl)pyridin-3-yl)cyclopropanamine; (trans)-2-(6-(3,5-bis(trifluoromethyl)phenyl)pyridin-3-yl)cyclopropanamine; N-(3-(5-((trans)-2-aminocyclopropyl)pyridin-2-yl)phenyl)acetamide; N-(3-(5-((trans)-2-aminocyclopropyl)pyridin-2-yl)phenyl)methanesulfonamide; (trans)-2-(6-(benzo[b]thiophen-2-yl)pyridin-3-yl)cyclopropanamine; (trans)-2-(6-(benzo[b]thiophen-3-yl)pyridin-3-yl)cyclopropanamine; 5-(5-((trans)-2-aminocyclopropyl)pyridin-2-yl)thiophene-2-carbonitrile; (trans)-2-(6-(4-methylthiophen-3-yl)pyridin-3-yl)cyclopropanamine; (trans)-2-(2-chloro-6-(3-(trifluoromethyl)phenyl)pyridin-3-yl)cyclopropanamine; (trans)-2-(2-(4-chlorophenyl)-6-(3-(trifluoromethyl)phenyl)pyridine-3-yl)cyclopropanamine; 4-(3-((trans)-2-aminocyclopropyl)-6-(3-(trifluoromethyl)phenyl)pyridin-2-yl)phenol; 4-(3-((trans)-2-aminocyclopropyl)-6-(3-(trifluoromethyl)phenyl)-pyridin-2-yl)benzamide; (trans)-2-(2-methyl-6-(3-(trifluoromethyl)phenyl)pyridin-3-yl)cyclopropanamine; 3-(5-((trans)-2-aminocyclopropyl)pyridin-2-yl)-5-hydroxybenzonitrile; (trans)-2-(6-(3,4-difluoro-5-methoxyphenyl)pyridin-3-yl)cyclopropanamine; 5-(5-((trans)-2-aminocyclopropyl)pyridin-2-yl)-2,3-difluorophenol; (trans)-2-(6-(3-chloro-4-fluoro-5-methoxyphenyl)pyridin-3-yl)cyclopropanamine; 5-(5-((trans)-2-aminocyclopropyl)pyridin-2-yl)-3-chloro-2-fluorophenol; (trans)-2-(6-(1H-indazol-6-yl)pyridin-3-yl)cyclopropanamine; (trans)-2-(6-(9H-carbazol-2-yl)pyridin-3-yl)cyclopropanamine; 6-(5-((trans)-2-aminocyclopropyl)pyridin-2-yl)indolin-2-one; 6-(5-((trans)-2-aminocyclopropyl)pyridin-2-yl)benzofuran-2(3H)-one; 4-(5-((trans)-2-aminocyclopropyl)pyridin-2-yl)pyridin-2(1H)-one; N-(3-(5-((trans)-2-aminocyclopropyl)pyridin-2-yl)phenyl)benzenesulfonamide; N-(3-(5-((trans)-2-aminocyclopropyl)pyridin-2-yl)phenyl)propane-2-sulfonamide; 4′-((trans)-2-aminocyclopropyl)-4-fluorobiphenyl-3-ol; 4′-((trans)-2-aminocyclopropyl)-5-chlorobiphenyl-3-ol; 4′-((trans)-2-aminocyclopropyl)-5-chloro-4-fluorobiphenyl-3-ol; N-(4′-((trans)-2-aminocyclopropyl)biphenyl-3-yl)benzenesulfonamide; N-(4′-((trans)-2-aminocyclopropyl)biphenyl-3-yl)propane-2-sulfonamide; N-(4′-((trans)-2-aminocyclopropyl)biphenyl-3-yl)methanesulfonamide; N-(2-(5-((trans)-2-aminocyclopropyl)pyridin-2-yl)phenyl)methanesulfonamide; 3-(5-((trans)-2-aminocyclopropyl)pyridin-2-yl)-4-methoxybenzonitrile; N-(4′-((trans)-2-aminocyclopropyl)biphenyl-2-yl)methanesulfonamide; 4′-((trans)-2-aminocyclopropyl)-6-methoxybiphenyl-3-carbonitrile; N-(4′-((trans)-2-aminocyclopropyl)-6-methoxybiphenyl-3-yl)methanesulfonamide; 4′-((trans)-2-aminocyclopropyl)-6-hydroxybiphenyl-3-carbonitrile; N-(4′-((trans)-2-aminocyclopropyl)-6-hydroxybiphenyl-3-yl)methanesulfonamide; 3-(5-((trans)-2-aminocyclopropyl)pyridin-2-yl)-4-hydroxybenzonitrile; N-(3-(5-((trans)-2-aminocyclopropyl)pyridin-2-yl)-4-hydroxyphenyl)methane-sulfonamide; N-(3-(5-((trans)-2-aminocyclopropyl)pyridin-2-yl)-5-(trifluoromethyl)phenyl)ethanesulfonamide; N-(3-(5-((trans)-2-aminocyclopropyl)pyridin-2-yl)-5-(trifluoromethyl)phenyl)methanesulfonamide; 3-(6-((trans)-2-aminocyclopropyl)pyridin-3-yl)phenol; (trans)-2-(5-(3-methoxyphenyl)pyridin-2-yl)cyclopropanamine; 4-(6-((trans)-2-aminocyclopropyl)pyridin-3-yl)phenol; 2-(6-((trans)-2-aminocyclopropyl)pyridin-3-yl)phenol; 2-(5-((trans)-2-aminocyclopropyl)thiophen-2-yl)phenol; 3-(5-((trans)-2-aminocyclopropyl)thiophen-2-yl)phenol; 4-(5-((trans)-2-aminocyclopropyl)thiophen-2-yl)phenol; 2-(5-((trans)-2-aminocyclopropyl)thiazol-2-yl)phenol; 3-(5-((trans)-2-aminocyclopropyl)thiazol-2-yl)phenol; 4-(5-((trans)-2-aminocyclopropyl)thiazol-2-yl)phenol; 2-(2-((trans)-2-aminocyclopropyl)thiazol-5-yl)phenol; 3-(2-((trans)-2-aminocyclopropyl)thiazol-5-yl)phenol; 2-(2-((trans)-2-aminocyclopropyl)thiazol-5-yl)phenol; 3-(2-((trans)-2-aminocyclopropyl)thiazol-5-yl)phenol; 3-(5-((trans)-2-aminocyclopropyl)pyrimidin-2-yl)phenol; 4-(5-((trans)-2-aminocyclopropyl)pyrimidin-2-yl)phenol; N-(3-(5-((trans)-2-aminocyclopropyl)pyridin-2-yl)-4-methoxyphenyl)methanesulfonamide; N-(4′-((trans)-2-aminocyclopropyl)-5-chloro-[1,1′-biphenyl]-3-yl)methanesulfonamide; N-(3-(5-((trans)-2-aminocyclopropyl)pyridin-2-yl)-5-chlorophenyl)methanesulfonamide; N-(4′-((trans)-2-aminocyclopropyl)-4-fluoro-[1,1′-biphenyl]-3-yl)methanesulfonamide; N-(5-(5-((trans)-2-aminocyclopropyl)pyridin-2-yl)-2-fluorophenyl)methanesulfonamide; N-(3-(5-((trans)-2-aminocyclopropyl)pyridin-2-yl)phenyl)ethanesulfonamide; N-(3-(5-((trans)-2-aminocyclopropyl)pyridin-2-yl)phenyl)-4-cyanobenzenesulfonamide; N-(3-(5-((trans)-2-aminocyclopropyl)pyridin-2-yl)phenyl)-3-cyanobenzenesulfonamide; N-(3-(5-((trans)-2-aminocyclopropyl)pyridin-2-yl)phenyl)-2-cyanobenzenesulfonamide; N-(3-(5-((trans)-2-aminocyclopropyl)pyridin-2-yl)-5-(trifluoromethyl)phenyl)-4-cyanobenzenesulfonamide; N-(4′-((trans)-2-aminocyclopropyl)-[1,1′-biphenyl]-3-yl)-1,1,1-trifluoromethanesulfonamide; 4′-((trans)-2-aminocyclopropyl)-6-hydroxy-[1,1′-biphenyl]-3-carbonitrile; 4′-((trans)-2-aminocyclopropyl)-[1,1′-biphenyl]-2-ol; 4′-((trans)-2-aminocyclopropyl)-3′-methoxy-[1,1′-biphenyl]-3-ol; N-(3-(5-((trans)-2-aminocyclopropyl)thiazol-2-yl)phenyl)-2-cyanobenzenesulfonamide; or a pharmaceutically acceptable salt or solvate thereof.

In other embodiments, LSD1 inhibitor compounds are selected from phenylcyclopropylamine derivatives, as described for example by Ogasawara et al. (2013, Angew. Chem. Int. Ed., 52: 8620-8624, which is hereby incorporated by reference herein in its entirety). Representative compounds of this type are represented by Formula XIV:

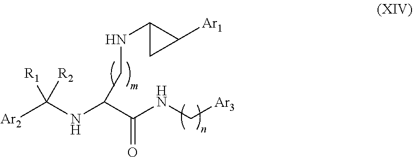

wherein Ar₁ is a 5 to 7 membered aryl or heteroaryl ring;
Ar₂ and Ar₃ are each independently selected from a 5 to 7 membered aryl or heteroaryl ring, optionally substituted with 1 to 3 substituents;
R₁ and R₂ are independently selected from hydrogen and hydroxyl or taken together R₁ and R₂ form ═O, ═S or ═NR₃;
R₃ is selected from hydrogen, —C₁₋₆alkyl or —OH;
m is an integer from 1 to 5; and
n is an integer from 1 to 3;
or a pharmaceutically acceptable salt thereof.

In particular embodiments of Formula XIV, one or more of the following applies:

Ar₁ is a six membered aryl or heteroaryl ring, especially phenyl, pyridine, pyrimidine, pyrazine 1,3,5-triazine, 1,2,4-trazine and 1,2,3-triazine, more especially phenyl;
Ar₂ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position;
Ar₃ is a six membered aryl or heteroaryl ring, especially phenyl, pyridine, pyrimidine, pyrazine 1,3,5-triazine, 1,2,4-trazine and 1,2,3-triazine, especially phenyl; especially where the six membered aryl or heteroaryl ring is optionally substituted with one optional substituent, especially in the 3 or 4 position.
Particular optional substituents for Ar₁ and Ar₂ include —C₁₋₆alkyl, —C₂₋₆alkenyl, —CH₂F, —CHF₂, —CF₃, halo, aryl, heteroaryl, —C(O)NHC₁₋₆alkyl, —C(O)NHC₁₋₆alkylNH₂, —C(O)-heterocyclyl, especially methyl, ethyl, propyl, butyl, t-butyl, —CH₂F, —CHF₂, —CH₃, Cl, F, phenyl, —C(O)NH(CH₂)₁₋₄NH₂ and —C(O)-heterocyclyl;
R₁ and R₂ taken together form ═O, ═S or ═NR₃, especially ═O or ═S, more especially ═O;
R₃ is H, —C₁₋₃alkyl or —OH, especially H, —CH₃ or —OH.
m is 2 to 5, especially 3 to 5, more especially 4,
n is 1 or 2, especially 1.

In some embodiments the compounds of Formula XIV are compounds of Formula XIVa:

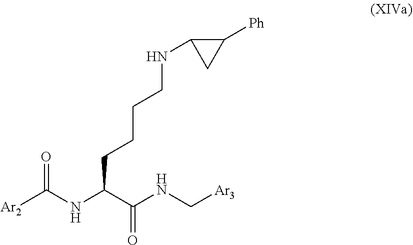

wherein Ar₂ and Ar₃ are as defined for Formula XIV.

Non-limiting compounds represented by Formula XIV include the following:

| Compound | Ar₂ | Ar₃ |
|---|---|---|
| 1b | phenyl | phenyl |
| 1c | 4-methylphenyl | phenyl |
| 1d | 4-t-butylphenyl | phenyl |
| 1e | 4-chlorophenyl | phenyl |
| 1f | 4-fluorophenyl | phenyl |
| 1g | 4-phenyl-phenyl | Phenyl |
| 1h | 4-trifluoromethylphenyl | Phenyl |
| 1i | 3-(2-aminoethylcarbamoyl)phenyl | Phenyl |
| 1j | 3-(piperazine-1-carbonyl)phenyl | Phenyl |
| 1k | 4-phenyl-phenyl | 4-methylphenyl |
| 1l | 4-phenyl-phenyl | 4-fluorophenyl |
| 1m | 4-phenyl-phenyl | 4-phenyl-phenyl |
| 1n | 4-phenyl-phenyl | 4-t-butylphenyl |
| 1o | 4-phenyl-phenyl | 3-methylphenyl |
| 1p | 4-phenyl-phenyl | 3-fluorophenyl |
| 1q | 4-phenyl-phenyl | 3-phenyl-phenyl |

The synthesis and inhibitory activity of the compounds of Formula (XIV) are described by Ogasawara et al. (2013, supra).

Other LSD1 inhibitors include, but are not limited to those, e.g., disclosed in Ueda et al. (2009, J. Am. Chem. Soc., 131(48): 17536-17537) including; Mimasu et al. (2010, Biochemistry, 49(30): 6494-6503).

Other phenylcyclopropylamine derivatives and analogs are found, e.g., in Kaiser et al. (1962), J. Med. Chem., 5: 1243-1265; Zirkle et al. (1962), J. Med. Chem., 1265-1284; U.S. Pat. Nos. 3,365,458; 3,471,522; and 3,532,749; Bolesov et al. (1974), Zhurnal Organicheskoi Khimii, 10(8): 1661-1669; and Russian Patent No. 230169 (19681030).

2.4 Inhibitors Identified by Screening Assays

Along with known LSD1 inhibitors, the invention also encompasses LSD1 inhibitors identified by any suitable screening assay. Accordingly, the present invention extends to methods of screening for inhibitory agents that are useful for inhibiting LSD1 and, in turn, for inhibiting immune checkpoints, particularly PD-L1 and/or PD-L2. In some embodiments, the screening methods comprise (1) contacting a preparation with a test agent, wherein the preparation comprises (i) a polypeptide comprising an amino acid sequence corresponding to at least a biologically active fragment of LSD1 or to a variant or derivative thereof; or (ii) a polynucleotide comprising a nucleotide sequence from which a transcript of an LSD1 gene or portion thereof is producible, or (iii) a polynucleotide comprising at least a portion of a genetic sequence (e.g. a transcriptional element) that regulates the expression of an LSD1 gene, which is operably linked to a reporter gene; and (2) detecting a change in the level or functional activity of the polypeptide, the polynucleotide or an expression product of the reporter gene, relative to a reference level or functional activity in the absence of the test agent. A detected reduction in the level and/or functional activity of the polypeptide, transcript or transcript portion or an expression product of the reporter gene, relative to a normal or reference level and/or functional activity in the absence of the test agent, indicates that the agent is useful for inhibiting immune checkpoints, particularly PD-L1 and/or PD-L2.

Inhibitors falling within the scope of the present invention include inhibitors of the level, functional activity or nuclear translocation of LSD1, including antagonistic antigen-binding molecules, and inhibitor peptide fragments, antisense molecules, ribozymes, RNAi molecules and co-suppression molecules as well as polysaccharide and lipopolysaccharide inhibitors of LSD1.

Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, desirably at least two of the functional groups. The candidate agent often comprises homocyclic carbon or heterocyclic structures or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule inhibitors of LSD1 are particularly advantageous. In this regard, small molecules are desirable because such molecules are more readily absorbed after oral administration, have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for modulatory agents according to the invention can be achieved by any suitable method. For example, the method may include contacting a cell expressing a polynucleotide corresponding to a gene that encodes LSD1 with an agent suspected of having the inhibitory activity and screening for the inhibition of the level or functional activity of LSD1, or the inhibition of the level of a transcript encoded by the polynucleotide, or the inhibition of the activity or expression of a downstream cellular target of the polypeptide or of the transcript (hereafter referred to as target molecules). Detecting such modulation can be achieved utilizing techniques including, but not restricted to, ELISA, cell-based ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which LSD1 is regulated or expressed may be naturally occurring in the cell which is the subject of testing or it may have been introduced into the host cell for the purpose of testing. In addition, the naturally-occurring or introduced polynucleotide may be constitutively expressed, thereby providing a model useful in screening for agents which down-regulate expression of an encoded product of the sequence wherein the down regulation can be at the nucleic acid or expression product level. Further, to the extent that a polynucleotide is introduced into a cell, that polynucleotide may comprise the entire coding sequence that codes for LSD1 or it may comprise a portion of that coding sequence (e.g., the active site of LSD1) or a portion that regulates expression of the corresponding gene that encodes LSD1 (e.g. an LSD1 promoter). For example, the promoter that is naturally associated with the polynucleotide may be introduced into the cell that is the subject of testing. In this instance, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

These methods provide a mechanism for performing high throughput screening of putative inhibitory agents such as proteinaceous or non-proteinaceous agents comprising synthetic, combinatorial, chemical and natural libraries. These methods will also facilitate the detection of agents which bind either the polynucleotide encoding LSD1 or which inhibit the expression of an upstream molecule, which subsequently inhibits the expression of the polynucleotide encoding LSD1. Accordingly, these methods provide a mechanism of detecting agents that either directly or indirectly inhibit the expression or activity of LSD1 according to the invention.

In alternative embodiments, test agents are screened using commercially available assays, illustrative examples of which include EpiQuik Histone Demethylase LSD1 Inhibitor Screening Assay Kit (Epigentek Group, Brooklyn, N.Y.) or the LSD1 Inhibitor Screening Assay Kit (Cayman Chemical Company, Ann Arbor, Mich.).

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

2.5 Novel Proteinaceous Molecule LSD1 Inhibitors

The present inventors have also conceived novel proteinaceous molecules that inhibit LSD1. In particular, the inventors have found that a proteinaceous molecule comprising, consisting or consisting essentially of a sequence corresponding to residues 108 to 118 of LSD1 inhibits LSD1, especially the nuclear translocation of LSD1. Such proteinaceous molecules inhibit formation and maintenance of cancer stem cell and non-cancer stem cell tumor cells. Thus, the inventors conceived that the proteinaceous molecules of the invention may be used for the treatment or prevention of a cancer. Furthermore, the inventors have conceived that the proteinaceous molecules of the invention may be useful in conditions involving PD-L1 and/or PD-L2 activity, such as an infection, or for enhancing an immune response. Accordingly, in another embodiment of the invention, the LSD1 inhibitor is an isolated or purified proteinaceous molecule comprising, consisting or consisting essentially of a sequence corresponding to residues 108 to 118 of LSD1.

The amino acid sequence of LSD1 (Uniprot No. 060341-1) is presented in SEQ ID NO: 5. Residues 108-118 are underlined in the sequence below.

[SEQ ID NO: 5]
MLSGKKAAAA AAAAAAAATG TEAGPGTAGG SENGSEVAAQ
PAGLSGPAEV GPGAVGERTP RKKEPPRASP PGGLAEPPGS
AGPQAGPTVV PGSATPMETG IAETPEGRRT SRRKRAKVEY
REMDESLANL SEDEYYSEEE RNAKAEKEKK LPPPPPQAPP
EEENESEPEE PSGVEGAAFQ SRLPHDRMTS QEAACFPDII
SGPQQTQKVF LFIRNRTLQL WLDNPKIQLT FEATLQQLEA
PYNSDTVLVH RVHSYLERHG LINFGIYKRI KPLPTKKTGK
VIIIGSGVSG LAAARQLQSF GMDVTLLEAR DRVGGRVATF
RKGNYVADLG AMVVTGLGGN PMAVVSKQVN MELAKIKQKC
PLYEANGQAV PKEKDEMVEQ EFNRLLEATS YLSHQLDFNV
LNNKPVSLGQ ALEVVIQLQE KHVKDEQIEH WKKIVKTQEE
LKELLNKMVN LKEKIKELHQ QYKEASEVKP PRDITAEFLV
KSKHRDLTAL CKEYDELAET QGKLEEKLQE LEANPPSDVY
LSSRDRQILD WHFANLEFAN ATPLSTLSLK HWDQDDDFEF
TGSHLTVRNG YSCVPVALAE GLDIKLNTAV RQVRYTASGC
EVIAVNTRST SQTFIYKCDA VLCTLPLGVL KQQPPAVQFV
PPLPEWKTSA VQRMGFGNLN KVVLCFDRVF WDPSVNLFGH
VGSTTASRGE LFLFWNLYKA PILLALVAGE AAGIMENISD
DVIVGRCLAI LKGIFGSSAV PQPKETVVSR WRADPWARGS
YSYVAAGSSG NDYDLMAQPI TPGPSIPGAP QPIPRLFFAG
EHTIRNYPAT VHGALLSGLR EAGRIADQFL GAMYTLPRQA
TPGVPAQQSP SM.

In some embodiments, the proteinaceous molecule is an isolated or purified proteinaceous molecule represented by Formula I:

Z₁RRTX₁RRKRAKVZ₂ (I)

wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof.

In some embodiments, “X₁” is selected from S and A.

In some embodiments, “X₁” is selected from S, A and modified forms thereof. In some embodiments, “X₁” is selected from S, A and S(PO₃).

In some embodiments, “X₁” is a modified form of S, especially S(PO₃).

In some embodiments, “Z₁” is a proteinaceous molecule represented by Formula II:

X₂X₃X₄ (II)

wherein:
“X₂” is absent or is a protecting moiety;
“X₃” is absent or is selected from any amino acid residue; and
“X₄” is selected from any amino acid residue.

In some embodiments, “X₃” is selected from basic amino acid residues including R, K and modified forms thereof. In some embodiments, “X₃” is R.

In some embodiments, “X₄” is selected from aromatic amino acid residues, including F, Y, W and modified forms thereof. In some embodiments, “X₄” is W.

In some embodiments, “Z₂” is absent.

In some embodiments, the isolated or purified proteinaceous molecule of Formula I comprises, consists or consists essentially of an amino acid sequence represented by SEQ ID NO: 1, 2 or 3:

[SEQ ID NO: 1]
RRTSRRKRAKV;
[SEQ ID NO: 2]
RRTARRKRAKV;
or
[SEQ ID NO: 3]
RWRRTARRKRAKV.

In particular embodiments, the isolated or purified proteinaceous molecule of Formula I comprises, consists or consists essentially of an amino acid sequence represented by SEQ ID NO: 1 or 2.

In some embodiments, the isolated or purified proteinaceous molecule of Formula I is other than a proteinaceous molecule consisting of the amino acid sequence of SEQ ID NO: 4:

[SEQ ID NO: 4]
EGRRTSRRKRAKVE.

The present invention also contemplates proteinaceous molecules that are variants of SEQ ID NO: 1, 2 or 3. Such “variant” proteinaceous molecules include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein.

Variant proteins encompassed by the present invention are biologically active, that is, they continue to possess the desired biological activity of the native protein. Such variants may result from, for example, genetic polymorphism or from human manipulation.

The proteinaceous molecules of SEQ ID NO: 1, 2 and/or 3 may be altered in various ways including amino acid substitutions, deletions, truncations, and insertions. Methods for such manipulations are generally known in the art. For example, amino acid sequence variants of SEQ ID NO: 1, 2 and/or 3 can be prepared by mutagenesis of nucleic acids encoding the amino acid sequence of SEQ ID NO: 1, 2 and/or 3. Methods for mutagenesis and nucleotide sequence alterations are well known in the art. See, for example, Kunkel (1985, Proc. Natl. Acad. Sci. USA. 82: 488-492), Kunkel et al. (1987, Methods in Enzymol, 154: 367-382), U.S. Pat. No. 4,873,192, Watson et al. (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of the proteinaceous molecules of SEQ ID NO: 1, 2 and/or 3. Recursive ensemble mutagenesis (REM), a technique which enhances the frequency of functional mutants in the libraries, can be used in combination with screening assays to identify active variants (Arkin and Yourvan (1992) Proc. Natl. Acad. Sci. USA 89: 7811-7815; Delgrave et al. (1993) Protein Engineering, 6: 327-331). Conservative substitutions, such as exchanging one amino acid with another having similar properties, may be desirable as discussed in more detail below.

Variant peptides or polypeptides of the invention may contain conservative amino acid substitutions at various locations along their sequence, as compared to a parent (e.g. naturally-occurring or reference) amino acid sequence, such as SEQ ID NO: 1, 2 and/or 3. A “conservative amino acid substitution” is one in which the amino acid residue is replaced with an amino acid residue having a similar side chain. Families of amino acid residues having similar side chains have been defined in the art as discussed in detail below.

The amino acid sequence of the proteinaceous molecules of the invention is defined in terms of amino acids of certain characteristics or sub-classes. Amino acid residues are generally sub-classified into major sub-classes as follows:

Acidic: The residue has a negative charge due to loss of a proton at physiological pH and the residue is attracted by aqueous solution so as to seek the surface positions in the conformation of a peptide in which it is contained when the peptide is in aqueous medium at physiological pH. Amino acids having an acidic side chain include glutamic amid and aspartic acid.

Basic: The residue has a positive charge due to association with protons at physiological pH or within one or two pH units thereof (e.g. histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residue is charged at physiological pH and, therefore, includes amino acids having acidic or basic side chains, such as glutamic acid, aspartic acid, arginine, lysine and histidine.

Hydrophobic: The residue is not charged at physiological pH and the residue is repelled by aqueous solution so as to seek the inner positions in the conformation of a peptide in which it is contained when the peptide is in aqueous medium at physiological pH. Amino acids having a hydrophobic side chain include tyrosine, valine, isoleucine, leucine, methionine, phenylalanine and tryptophan.

Neutral/polar: The residues are not charged at physiological pH but the residue is not sufficiently repelled by aqueous solutions so that it would seek inner positions in the conformation of a peptide in which it is contained when the peptide is in aqueous medium at physiological pH. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g. PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (1992), Science, 256(5062): 1443-1445), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small amino acid residues are, of course, always non-aromatic. Dependent on their structural properties, amino acid residues may fall in two or more classes. For the naturally-occurring protein amino acids, sub-classification according to this scheme is presented in Table 1.

TABLE 1
Amino Acid Sub-Classification

| Sub-classes | Amino Acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Nonpolar/ | Alanine, Glycine, Isoleucine, Leucine, Methionine, |
| neutral | Phenylalanine, Proline, Tryptophan, Valine |
| Polar/neutral | Asparagine, Histidine, Glutamine, Cysteine, Serine, |
| | Threonine, Tyrosine |
| Polar/negative | Aspartic acid, Glutamic acid |
| Polar/positive | Lysine, Arginine |
| Polar/large | Asparagine, Glutamine |
| Polar | Arginine, Asparagine, Aspartic acid, Cysteine, Glutamic |
| | acid, Glutamine, Histidine, Lysine, Serine, Threonine, |
| | Tyrosine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartic acid with a glutamic acid, a threonine with a serine, or a similar replacement of an amino acid with a structurally related amino acid will not have a major effect on the properties of the resulting variant peptide useful in the invention. Whether an amino acid change results in a proteinaceous molecule that inhibits LSD1 can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary and Preferred Amino Acid Substitutions

| Original | | |
|---|---|---|
| Residue | Exemplary Substitutions | Preferred Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Nle | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Nle | Leu |

Where Nle is used to refer to norleucine.

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, Biochemistry, third edition, Wm.C. Brown Publishers (1993).

Thus, a predicted non-essential amino acid residue in a peptide of the invention is typically replaced with another amino acid residue from the same side chain family. Alternatively, mutations can be introduced randomly along all or part of the coding sequence of a peptide of the invention, such as by saturation mutagenesis, and the resultant mutants can be screened for an activity of the parent polypeptide, as described for example herein, to identify mutants which retain that activity. Following mutagenesis of the coding sequences, the encoded peptide can be expressed recombinantly and its activity determined. A “non-essential” amino acid residue is a residue that can be altered from the wild-type sequence of an embodiment peptide of the invention without abolishing or substantially altering one or more of its activities. Suitably, the alteration does not substantially alter one of these activities, for example, the activity is at least 20%, 40%, 60%, 70% or 80% of that of the wild-type. By contrast, an “essential” amino acid residue is a residue that, when altered from the wild-type sequence of an embodiment peptide of the invention, results in abolition of an activity of the parent molecule such that less than 20% of the wild-type activity is present. For example, such essential amino acid residues include Arg (or modified form thereof) at position 2, Arg (or modified form thereof) at positions 2, 3, 6, 7 and 9, Thr (or modified form thereof) at position 4, Lys (or modified form thereof) at positions 8 and 11, Ala (or modified form thereof) at position 10, and Val (or modified form thereof) at position 12, relative to the numbering of Formula (I) commencing at Z₁.

Accordingly, the present invention also contemplates variants of the proteinaceous molecules of SEQ ID NO 1, 2 and/or 3 of the invention, wherein the variants are distinguished from the parent sequence by the addition, deletion, or substitution of one or more amino acid residues. In general, variants will display at least about 80%, 81%, 82%, 83%, 84%, 85%, 86%, 87%, 88%, 89%, 90%, 91%, 92%, 93%, 94%, 95%, 96%, 97%, 98%, 99% sequence similarity to a parent or reference proteinaceous molecule sequence as, for example, set forth in SEQ ID NO: 1, 2 or 3, as determined by sequence alignment programs described elsewhere herein using default parameters. Desirably, variants will have at least 80%, 81%, 82%, 83%, 84%, 85%, 86%, 87%, 88%, 89%, 90%, 91%, 92%, 93%, 94%, 95%, 96%, 97%, 98%, 99% sequence identity to a parent or reference peptide sequence as, for example, set forth in SEQ ID NO: 1, 2 or 3, as determined by sequence alignment programs described herein using default parameters. Variants of SEQ ID NO: 1, 2 and/or 3, which fall within the scope of a variant peptide of the invention, may differ from the parent molecule generally by at least 1, but by less than 4, 3, 2 or 1 amino acid residue(s). In some embodiments, a variant peptide of the invention differs from the corresponding sequence in SEQ ID NO: 1, 2 or 3 by at least 1, but by less than 4, 3, 2 or 1 amino acid residue(s). In some embodiments, the amino acid sequence of the variant peptide of the invention comprises Arg (or modified form thereof) at position 2, Arg (or modified form thereof) at positions 2, 3, 6, 7 and 9, Thr (or modified form thereof) at position 4, Lys (or modified form thereof) at positions 8 and 11, Ala (or modified form thereof) at position 10, and Val (or modified form thereof) at position 12, relative to the numbering of Formula (I) commencing at Z₁. In some embodiments, the amino acid sequence of the variant peptide of the invention comprises the proteinaceous molecule of Formula I. In particular embodiments, the variant peptide of the invention inhibits LSD1, particularly LSD1 nuclear translocation.

If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution.

In some embodiments, calculations of sequence similarity or sequence identity between sequences are performed as follows:

To determine the percent identity of two amino acid sequences or of two nucleic acid sequences, the sequences are aligned for optimal comparison purposes (e.g. gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 40%, more usually at least 50% or 60%, and even more usually at least 70%, 80%, 90% or 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e. conservative substitution) at the corresponding position in the second sequence, then the molecules are similar at that position.

The percent identity between the two sequences is a function of the number of identical amino acid residues shared by the sequences at individual positions, taking into account the number of gaps and the length of each gap, which need to be introduced for optimal alignment of the two sequences. By contrast, the percent similarity between the two sequences is a function of the number of identical and similar amino acid residues shared by the sequences at individual positions, taking into account the number of gaps and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wünsch (1970, J. Mol. Biol., 48: 444-453) algorithm which has been incorporated into the GAP program in the GCG software package (Devereaux, et al. (1984) Nucleic Acids Research, 12: 387-395), using either a Blosum 62 matrix or a PAM250 matrix, and a gap weight of 16, 14, 12, 10, 8, 6, or 4 and a length weight of 1, 2, 3, 4, 5, or 6. In some embodiments, the percent identity or similarity between amino acid sequences can be determined using the algorithm of Meyers and Miller (1989, Cabios, 4: 11-17) which has been incorporated into the ALIGN program (version 2.0), using a PAM120 weight residue table, a gap length penalty of 12 and a gap penalty of 4.

The present invention also contemplates an isolated, synthetic or recombinant peptide that is encoded by a polynucleotide sequence that hybridizes under stringency conditions as defined herein, especially under medium, high or very high stringency conditions, preferably under high or very high stringency conditions, to a polynucleotide sequence encoding the peptides of SEQ ID NO: 1, 2 and/or 3 or the non-coding strand thereof. The invention also contemplates an isolated nucleic acid molecule comprising a polynucleotide sequence that hybridizes under stringency conditions as defined herein, especially under medium, high or very high stringency conditions, preferably under high or very high stringency conditions, to a polynucleotide sequence encoding the peptides of SEQ ID NO: 1, 2 and/or 3 or the non-coding strand thereof.

As used herein, the term “hybridizes under stringency conditions” describes conditions for hybridization and washing and may encompass low stringency, medium stringency, high stringency and very high stringency conditions.

Guidance for performing hybridization reactions can be found in Ausubel, et al. (1998) Current Protocols in Molecular Biology (John Wiley and Sons, Inc.), in particular sections 6.3.1-6.3.6. Both aqueous and non-aqueous methods can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% sodium dodecyl sulfate (SDS) for hybridization at 65° C., and (i) 2× sodium chloride/sodium citrate (SSC), 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6×SSC at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In some aspects of the present invention, there is provided an isolated, synthetic or recombinant peptide of the invention that is encoded by a polynucleotide sequence that hybridizes under high stringency conditions to a polynucleotide sequence encoding the peptides of SEQ ID NO: 1, 2 and/or 3 or the non-coding strand thereof. In certain embodiments, the isolated, synthetic or recombinant peptide of the invention is encoded by a polynucleotide sequence that hybridizes under very high stringency conditions to a polynucleotide sequence encoding the peptides of SEQ ID NO: 1, 2 and/or 3 or the non-coding strand thereof. One embodiment of very high stringency conditions includes hybridizing 0.5 M sodium phosphate, 7% SDS at 65° C., followed by one or more washes at 0.2×SSC, 1% SDS at 65° C. In some embodiments, the amino acid sequence of the variant peptide of the invention comprises Arg (or modified form thereof) at position 2, Arg (or modified form thereof) at positions 2, 3, 6, 7 and 9, Thr (or modified form thereof) at position 4, Lys (or modified form thereof) at positions 8 and 11, Ala (or modified form thereof) at position 10, and Val (or modified form thereof) at position 12, relative to the numbering of Formula (I) commencing at Z₁. In some embodiments, the amino acid sequence of the variant peptide of the invention comprises the proteinaceous molecule of Formula I. In particular embodiments, the variant peptide of the invention inhibits LSD1, particularly LSD1 nuclear translocation.

Other stringency conditions are well known in the art and a person skilled in the art will recognize that various factors can be manipulated to optimize the specificity of the hybridization. Optimization of the stringency of the final washes can serve to ensure a high degree of hybridization. For detailed examples, see Ausubel, et al. (1998) Current Protocols in Molecular Biology (John Wiley and Sons, Inc.), in particular pages 2.10.1 to 2.10.16 and Sambrook, et al. (1989) Molecular Cloning: A Laboratory Manual (Cold Spring Harbour Press), in particular Sections 1.101 to 1.104.

While stringent washes are typically carried out at temperatures from about 42° C. to 68° C., a person skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel, et al. (1998) Current Protocols in Molecular Biology (John Wiley and Sons, Inc.) at page 2.10.8). In general, the Tₘ of a perfectly matched duplex of DNA may be predicted as an approximation by the formula:

Tₘ=81.5+16.6(log₁₀M)+0.41(% G+C)−0.63(% formamide)−(600/length)

wherein: M is the concentration of Na⁺, preferably in the range of 0.01 M to 0.4 M; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g. a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrrolidone and 0.1% BSA), 0.1% SDS and 200 mg/mL denatured salmon sperm DNA) containing labeled probe. The membrane is then subjected to two sequential medium stringency washes (i.e. 2×SSC, 0.1% SDS for 15 min at 45° C., followed by 2×SSC, 0.1% SDS for 15 min at 50° C.), followed by two sequential higher stringency washes (i.e. 0.2×SSC, 0.1% SDS for 12 min at 55° C. followed by 0.2×SSC and 0.1% SDS solution for 12 min at 65-68° C.

The proteinaceous molecules of the present invention also encompass peptides comprising amino acids with modified side chains, incorporation of unnatural amino acid residues and/or their derivatives during peptide synthesis and the use of cross-linkers and other methods which impose conformational constraints on the peptides of the invention. Examples of side chain modifications include modifications of amino groups, such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with sodium borohydride; reductive alkylation by reaction with an aldehyde followed by reduction with sodium borohydride; and trinitrobenzylation of amino groups with 2,4,6-trinitrobenzene sulfonic acid (TNBS).

The carboxyl group may be modified by carbodiimide activation through O-acylisourea formation followed by subsequent derivatization, for example, to a corresponding amide.

The guanidine group of arginine residues may be modified by formation of heterocyclic condensation products with reagents such as 2,3-butanedione, phenylglyoxal and glyoxal.

Tryptophan residues may be modified, for example, by alkylation of the indole ring with 2-hydroxy-5-nitrobenzyl bromide or sulfonyl halides, or by oxidation with N-bromosuccinimide.

Tyrosine residues may be modified by nitration with tetranitromethane to form 3-nitrotyrosine derivatives.

Examples of incorporating unnatural amino acids and derivatives during peptide synthesis include, but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine, selenocysteine, O-phosphoserine, and α,α-difluoromethylenephosphonoserine and/or D-isomers of amino acids. A list of unnatural amino acids contemplated by the present invention is shown in Table 3.

TABLE 3
Exemplary Unnatural Amino Acids
Non-Conventional Amino Acids

| | |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-naphthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvaline | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | O-phospho-L-serine [abbreviated as |
| amino)cyclopropane | S(PO₃)] |
| O-phospho-D-serine | α,α-difluoromethylenephosphonoserine |
| | [abbreviated as S(CF₂PO₃)] |

Although the proteinaceous molecules of the invention may inherently permeate membranes, membrane permeation may further be increased by the conjugation of a membrane permeating moiety to the proteinaceous molecule. Accordingly, in some embodiments the proteinaceous molecules of the invention comprise at least one membrane permeating moiety. The membrane permeating moiety may be conjugated at any point of the proteinaceous molecule. Suitable membrane permeating moieties include lipid moieties, cholesterol and proteins, such as cell-penetrating peptides and polycationic peptides; especially lipid moieties.

Suitable cell penetrating peptides may include the peptides described in, for example, US 20090047272, US 20150266935 and US 20130136742. Accordingly, suitable cell penetrating peptides may include, but are not limited to, basic poly(Arg) and poly(Lys) peptides and basic poly(Arg) and poly(Lys) peptides containing non-natural analogues of Arg and Lys residues such as YGRKKRPQRRR (HIV TAT₄₇₋₅₇; SEQ ID NO: 6), RRWRRWWRRWWRRWRR (W/R; SEQ ID NO: 7), CWK₁₈ (AlkCWK₁₈; SEQ ID NO: 8), K₁₈WCCWK₁₈ (Di-CWK₁₈; SEQ ID NO: 9), WTLNSAGYLLGKINLKALAALAKKIL (Transportan; SEQ ID NO: 10), GLFEALEELWEAK (DipaLytic; SEQ ID NO: 11), K₁₆GGCRGDMFGCAK₁₆RGD (K₁₆RGD; SEQ ID NO: 12), K₁₆GGCMFGCGG (P1; SEQ ID NO: 13), K₁₆ICRRARGDNPDDRCT (P2; SEQ ID NO: 14), KKWKMRRNQFWVKVQRbAK (B) bA (P3; SEQ ID NO: 15), VAYISRGGVSTYYSDTVKGRFTRQKYNKRA (P3a; SEQ ID NO: 16), IGRIDPANGKTKYAPKFQDKATRSNYYGNSPS (P9.3; SEQ ID NO: 17), KETWWETWWTEWSQPKKKRKV (Pep-1; SEQ ID NO: 18), PLAEIDGIELTY (Plae; SEQ ID NO: 19), K₁₆GGPLAEIDGIELGA (Kplae; SEQ ID NO: 20), K₁₆GGPLAEIDGIELCA (cKplae; SEQ ID NO: 21), GALFLGFLGGAAGSTMGAWSQPKSKRKV (MGP; SEQ ID NO: 22), WEAK(LAKA)₂-LAKH(LAKA)₂LKAC (HA2; SEQ ID NO: 23), (LARL)₆NHCH₃ (LARL4₆; SEQ ID NO: 24), KLLKLLLKLWLLKLLL (Hel-11-7; SEQ ID NO: 25), (KKKK)₂GGC (KK; SEQ ID NO: 26), (KWKK)₂GCC (KWK; SEQ ID NO: 27), (RWRR)₂GGC (RWR; SEQ ID NO: 28), PKKKRKV (SV40 NLS7; SEQ ID NO: 29), PEVKKKRKPEYP (NLS12; SEQ ID NO: 30), TPPKKKRKVEDP (NLS12a; SEQ ID NO: 31), GGGGPKKKRKVGG (SV40 NLS13; SEQ ID NO: 32), GGGFSTSLRARKA (AV NLS13; SEQ ID NO: 33), CKKKKKKSEDEYPYVPN (AV RME NLS17; SEQ ID NO: 34), CKKKKKKKSEDEYPYVPNFSTSLRARKA (AV FP NLS28; SEQ ID NO: 35), LVRKKRKTEEESPLKDKDAKKSKQE (SV40 N1 NLS24; SEQ ID NO: 36), and K₉K₂K₄K₈GGK₅ (Loligomer; SEQ ID NO: 37); HSV-1 tegument protein VP22; HSV-1 tegument protein VP22r fused with nuclear export signal (NES); mutant B-subunit of Escherichia coli enterotoxin EtxB (H57S); detoxified exotoxin A (ETA); the protein transduction domain of the HIV-1 Tat protein, GRKKRRQRRRPPQ (SEQ ID NO: 38); the Drosophila melanogaster Antennapedia domain Antp (amino acids 43-58), RQIKIWFQNRRMKWKK (SEQ ID NO: 39); Buforin II, TRSSRAGLQFPVGRVHRLLRK (SEQ ID NO: 40); hClock-(amino acids 35-47) (human Clock protein DNA-binding peptide), KRVSRNKSEKKRR (SEQ ID NO: 41); MAP (model amphipathic peptide), KLALKLALKALKAALKLA (SEQ ID NO: 42); K-FGF, AAVALLPAVLLALLAP (SEQ ID NO: 43); Ku70-derived peptide, comprising a peptide selected from the group comprising VPMLKE (SEQ ID NO: 44), VPMLK (SEQ ID NO: 45), PMLKE (SEQ ID NO: 46) or PMLK (SEQ ID NO: 47); Prion, Mouse Prpe (amino acids 1-28), MANLGYWLLALFVTMWTDVGLCKKRPKP (SEQ ID NO: 48); pVEC, LLIILRRRIRKQAHAHSK (SEQ ID NO: 49); Pep-I, KETWWETWWTEWSQPKKKRKV (SEQ ID NO: 50); SynBI, RGGRLSYSRRRFSTSTGR (SEQ ID NO: 51); Transportan, GWTLNSAGYLLGKINLKALAALAKKIL (SEQ ID NO: 52); Transportan-10, AGYLLGKINLKALAALAKKIL (SEQ ID NO: 53); CADY, Ac-GLWRALWRLLRSLWRLLWRA-cysteamide (SEQ ID NO: 54); Pep-7, SDLWEMMMVSLACQY (SEQ ID NO: 55); HN-1, TSPLNIHNGQKL (SEQ ID NO: 56); VT5, DPKGDPKGVTVTVTVTVTGKGDPKPD (SEQ ID NO: 57); or pISL, RVIRVWFQNKRCKDKK (SEQ ID NO: 58).

In preferred embodiments, the membrane permeating moiety is a lipid moiety, such as a C₁₀-C₂₀ fatty acyl group, especially octadecanoyl (stearoyl; C₁₈), hexadecanoyl (palmitoyl; C₁₆) or tetradecanoyl (myristoyl; C₁₄); most especially tetradecanoyl. In preferred embodiments, the membrane permeable moiety is conjugated (attached) to the N- or C-terminal amino acid residue or through the amine of a lysine side-chain of the proteinaceous molecule, especially the N-terminal amino acid residue of the proteinaceous moiety.

For particular uses and methods of the invention, proteinaceous molecules with high levels of stability may be desired, for example, to increase the half-life of the proteinaceous molecule in a subject. Thus, in some embodiments, the proteinaceous molecules of the invention comprise a stabilizing moiety, which is also referred to herein as a “protecting moiety”. The stabilizing moiety may be conjugated at any point on the proteinaceous molecule. Suitable stabilizing moieties include polyethylene glycol (PEG) or a capping moiety, including an acetyl group, pyroglutamate or an amino group. In preferred embodiments, the acetyl group and/or pyroglutamate are conjugated to the N-terminal amino acid residue of the proteinaceous molecule. In particular embodiments, the N-terminus of the proteinaceous molecule is a pyroglutamide or acetamide. In preferred embodiments, the amino group is conjugated to the C-terminal amino acid residue of the proteinaceous molecule. In particular embodiments, the proteinaceous molecule of the invention has a primary amide at the C-terminus. In preferred embodiments, the PEG is conjugated to the N-terminal or C-terminal amino acid residue of the proteinaceous molecule or through the amine of a lysine side-chain, especially through the N-terminal amino acid residue or through the amine of a lysine side-chain.

In preferred embodiments, the proteinaceous molecules of the invention have a primary amide or a free carboxyl group (C-terminal acid) at the C-terminus and a primary amine at the N-terminus.

In some embodiments, the proteinaceous molecules of the present invention are cyclic peptides. Without wishing to be bound by theory, cyclization of peptides is thought to decrease the susceptibility of the peptides to degradation. In particular embodiments, the proteinaceous molecules are cyclized using N-to-C cyclization (head to tail cyclization), preferably through an amide bond. Such peptides do not possess N- or C-terminal amino acid residues. In particular embodiments, the proteinaceous molecules of the invention have an amide-cyclized peptide backbone. In other embodiments, the proteinaceous molecules of the invention are cyclized using side-chain to side-chain cyclization, preferably through a disulfide bond or a lactam bridge.

In some embodiments, the N- and C-termini are linked using a linking moiety. The linking moiety may be a peptide linker such that cyclization produces an amide-cyclized peptide backbone. Variation within the peptide sequence of the linking moiety is possible, such that the linking moiety may be modified to alter the physicochemical properties of the proteinaceous molecules and potentially reduce side effects of the proteinaceous molecules of the invention or otherwise improve the therapeutic use of the proteinaceous molecules, for example, by improving stability. The linking moiety will be of suitable length to span the distance between the N- and C-termini of the peptide without substantially altering the structural conformation of the proteinaceous molecule, for example, a peptidic linking moiety may be between 2 and 10 amino acid residues in length. In some embodiments, longer or shorter peptidic linking moieties may be required.

The proteinaceous molecules of the invention may be in the form of salts or prodrugs. The salts of the proteinaceous molecules of the present invention are preferably pharmaceutically acceptable, but it will be appreciated that non-pharmaceutically acceptable salts also fall within the scope of the present invention.

The proteinaceous molecules of the present invention may be in crystalline form and/or in the form of solvates, for example, hydrates. Solvation may be performed using methods known in the art.

In some embodiments, the proteinaceous molecules of the invention selectively inhibit LSD1 over at least one other LSD or another enzyme such as a MAO. In some embodiments, the proteinaceous molecules of the invention selectively inhibit LSD1 over the other LSD subtypes and MAOs. In some embodiments, the proteinaceous molecules of the invention exhibit LSD1 selectivity of greater than about 2-fold, 5-fold, 10-fold, 20-fold, 50-fold or greater than about 100-fold with respect to inhibition of another LSD or MAO. In other embodiments, selective molecules display at least 50-fold greater inhibition towards LSD1 than another LSD or MAO. In further embodiments, selective molecules display at least 100-fold greater inhibition towards LSD1 than towards another LSD or MAO. In still further embodiments, selective molecules display at least 500-fold greater inhibition towards LSD1 than towards another LSD or MAO. In yet further embodiments, selective molecules display at least 100-fold greater inhibition towards LSD1 than towards another LSD or MAO. In some embodiments, the proteinaceous molecules of the invention are non-selective LSD1 inhibitors.

The present invention also contemplates nucleic acid molecules which encode a proteinaceous molecule of the invention. Thus, in a further aspect of the present invention, there is provided an isolated nucleic acid molecule comprising a polynucleotide sequence that encodes a proteinaceous molecule of the invention or is complementary to a polynucleotide sequence that encodes a proteinaceous molecule of the invention, such as the proteinaceous molecule of Formula I; SEQ ID NO: 1, 2 or 3; or variant proteinaceous molecule as described herein.

In some embodiments, the proteinaceous molecule encoded by the polynucleotide sequence is other than a proteinaceous molecule consisting of the amino acid sequence of SEQ ID NO: 4.

The isolated nucleic acid molecules of the present invention may be DNA or RNA. When the nucleic acid is in DNA form, it may be genomic DNA or cDNA. RNA forms of the nucleic acid molecules of the present invention are generally mRNA.

Although the nucleic acid molecules are typically isolated, in some embodiments the nucleic acid molecules may be integrated into, ligated to, or otherwise fused or associated with other genetic molecules, such as an expression vector. Generally an expression vector includes transcriptional and translational regulatory nucleic acid operably linked to the polynucleotide sequence. Accordingly, in another aspect of the invention, there is provided an expression vector comprising a polynucleotide sequence that encodes a proteinaceous molecule of the invention, such as the proteinaceous molecule of Formula I; SEQ ID NO: 1, 2 or 3; or variant proteinaceous molecule as described herein.

In some embodiments, the proteinaceous molecules of the invention may be produced inside a cell by introduction of one or more expression constructs, such as an expression vector, that comprise a polynucleotide sequence that encodes a proteinaceous molecule of the invention.

The invention contemplates recombinantly producing the proteinaceous molecules of the invention inside a host cell, such as a mammalian cell (e.g. Chinese hamster ovary (CHO) cell, mouse myeloma (NSO) cell, baby hamster kidney (BHK) cell or human embryonic kidney (HEK293) cell), yeast cell (e.g. Pichia pastoris cell, Saccharomyces cerevisiae cell, Schizosaccharomyces pombe cell, Hansenula polymorpha cell, Kluyveromyces lactis cell, Yarrowia lipolytica cell or Arxula adeninivorans cell), or bacterial cell (e.g. Escherichia coli cell, Corynebacterium glutamicum or Pseudomonas fluorescens cell).

For therapeutic applications, the invention also contemplates producing the proteinaceous molecules of the invention in vivo inside an LSD1, PKC-0, PD-L1 and/or PD-L2 overexpressing cell, especially an LSD1 overexpressing cell, such as a vertebrate cell, particularly a mammalian or avian cell, especially a mammalian cell.

As described, for example, in U.S. Pat. No. 5,976,567, the expression of natural or synthetic nucleic acids is typically achieved by operably linking a polynucleotide sequence encoding a proteinaceous molecule of the invention to a regulatory element (e.g. a promoter, which may be either constitutive or inducible), suitably incorporating the construct into an expression vector and introducing the vector into a suitable host cell. Typical vectors contain transcription and translation terminators, transcription and translation initiation sequences and promoters useful for regulation of the expression of the nucleic acid. The vectors optionally comprise generic expression cassettes containing at least one independent terminator sequence, sequences permitting replication of the cassette in eukaryotes, prokaryotes or both, (e.g. shuttle vectors) and selection markers for both prokaryotic and eukaryotic systems. Vectors may be suitable for replication and integration in prokaryotes, eukaryotes, or both. See, Giliman and Smith (1979), Gene, 8: 81-97; Roberts et al. (1987) Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), Molecular Cloning—a Laboratory Manual (2nd ed.) Vol. 1-3, Cold Spring Harbor Laboratory, Cold Spring Harbor Press, N.Y.; and Ausubel et al., (1994) Current Protocols in Molecular Biology, eds., Current Protocols, a joint venture between Greene Publishing Associates, Inc. and John Wiley & Sons, Inc. (Supplement).

Expression vectors containing regulatory elements from eukaryotic viruses such as retroviruses are typically used for expression of nucleic acid sequences in eukaryotic cells. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000) Curr. Opin. Biotechnol., 11(2): 205-208; Vigna and Naldini (2000) J. Gene Med., 2(5): 308-316; Kay et al. (2001) Nat. Med., 7(1): 33-40; Athanasopoulos et al. (2000) Int. J. Mol. Med., 6(4): 363-375; and Walther and Stein (2000) Drugs, 60(2): 249-271.

The polypeptide or peptide-encoding portion of the expression vector may comprise a naturally-occurring sequence or a variant thereof, which has been engineered using recombinant techniques. In one example of a variant, the codon composition of a polynucleotide encoding a proteinaceous molecule of the invention is modified to permit enhanced expression of the proteinaceous molecule of the invention in a mammalian host using methods that take advantage of codon usage bias, or codon translational efficiency in specific mammalian cell or tissue types as set forth, for example, in International Publications WO 99/02694 and WO 00/42215. Briefly, these latter methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the cell in which it is introduced such that the proteinaceous molecule of the invention is expressible by the cell. The expression vector is introduced into the cell by any suitable means which will be dependent on the particular choice of expression vector and cell employed. Such means of introduction are well-known to those skilled in the art. For example, introduction can be effected by use of contacting (e.g. in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g. Lipofectin®, Lipofectamine™, and the like, supplied by Invitrogen Waltham Mass., USA).

The proteinaceous molecules of the present invention may be prepared using recombinant DNA techniques or by chemical synthesis.

In some embodiments, the proteinaceous molecules of the present invention are prepared using standard peptide synthesis methods, such as solution synthesis or solid phase synthesis. The chemical synthesis of the proteinaceous molecules of the invention may be performed manually or using an automated synthesizer. For example, the linear peptides may be synthesized using solid phase peptide synthesis using either Boc or Fmoc chemistry, as described in Merrifield (1963) J Am Chem Soc, 85(14): 2149-2154; Schnolzer, et al. (1992) Int J Pept Protein Res, 40: 180-193; Ensenat-Waser, et al. (2002) IUBMB Life, 54:33-36; WO 2002/010193 and Cardosa, et al. (2015) Mol Pharmacol, 88(2): 291-303. Following deprotection and cleavage from the solid support, the linear peptides are purified using suitable methods, such as preparative chromatography.

In other embodiments, the proteinaceous molecules of the invention may be cyclized. Cyclization may be performed using several techniques, for example, as described in Davies (2003) J Pept Sci, 9: 471-501.

In some embodiments, the proteinaceous molecules of the present invention are prepared using recombinant DNA techniques. For example, the proteinaceous molecules of the invention may be prepared by a procedure including the steps of: (a) preparing a construct comprising a polynucleotide sequence that encodes the proteinaceous molecule of the invention and that is operably linked to a regulatory element; (b) introducing the construct into a host cell; (c) culturing the host cell to express the polynucleotide sequence to thereby produce the encoded proteinaceous molecule of the invention; and (d) isolating the proteinaceous molecule of the invention from the host cell. The proteinaceous molecule of the present invention may be prepared recombinantly using standard protocols, for example, as described in Klint, et al. (2013) PLOS One, 8(5): e63865; Sambrook, et al. (1989) Molecular Cloning: A Laboratory Manual (Cold Spring Harbour Press), in particular Sections 16 and 17; Ausubel, et al. (1998) Current Protocols in Molecular Biology (John Wiley and Sons, Inc.), in particular Chapters 10 and 16; and Coligan, et al. (1997) Current Protocols in Protein Science (John Wiley and Sons, Inc.), in particular Chapters 1, 5 and 6.

### 3. Pharmaceutical Compositions

In accordance with the present invention, the LSD1 inhibitors are useful in compositions and methods for the inhibition of immune checkpoints, particularly the inhibition of PD-L1 and/or PD-L2. The proteinaceous molecules of the invention are also useful in compositions and methods for the treatment or prevention of a condition involving LSD1, PKC, PD-L1 and/or PD-L2 overexpression, such as a cancer or infection. Thus, in some embodiments, the LSD1 inhibitors may be in the form of a pharmaceutical composition, wherein the pharmaceutical composition comprises an LSD1 inhibitor and a pharmaceutically acceptable carrier or diluent. In some embodiments, the LSD1 inhibitor is a proteinaceous molecule of the invention.

The LSD1 inhibitor may be formulated into the pharmaceutical composition as a neutral or salt form.

As will be appreciated by those skilled in the art, the choice of pharmaceutically acceptable carrier or diluent will be dependent on the route of administration and on the nature of the condition and subject to be treated. The particular carrier or delivery system and route of administration may be readily determined by a person skilled in the art. The carrier or delivery system and route of administration should be carefully selected to ensure that the activity of the LSD1 inhibitor is not depleted during preparation of the formulation and the LSD1 inhibitor is able to reach the site of action intact. The pharmaceutical compositions of the invention may be administered through a variety of routes including, but not limited to, oral, rectal, topical, intranasal, intraocular, transmucosal, intestinal, enteral, intramuscular, subcutaneous, intramedullary, intrathecal, intraventricular, intracerebral, intravaginal, intravesical, intravenous or intraperitoneal administration.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the LSD1 inhibitors using conventional approaches. Techniques for formulation and administration may be found in, for example, Remington (1980) Remington's Pharmaceutical Sciences, Mack Publishing Co., Easton, Pa., latest edition.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulphite; metal chelators such as ethylene diamine tetraacetic acid (EDTA); cysteine hydrochloride; sodium bisulfite; sodium metabisulfite; sodium sulfite; ascorbyl palmitate; lecithin; propyl gallate; and alpha-tocopherol.

For injection, the LSD1 inhibitors may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the LSD1 inhibitors can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the LSD1 inhibitors in water-soluble form. Additionally, suspensions of the LSD1 inhibitors may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the compounds to allow for the preparation of highly concentrated solutions.

Sterile solutions may be prepared by combining the active compounds in the required amount in the appropriate solvent with other excipients as described above as required, followed by sterilization, such as filtration. Generally, dispersions are prepared by incorporating the various sterilized active compounds into a sterile vehicle which contains the basic dispersion medium and the required excipients as described above. Sterile dry powders may be prepared by vacuum- or freeze-drying a sterile solution comprising the active compounds and other required excipients as described above.

Pharmaceutical preparations for oral use can be obtained by combining the LSD1 inhibitors with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The LSD1 inhibitors may be incorporated into modified-release preparations and formulations, for example, polymeric microsphere formulations, and oil- or gel-based formulations.

In particular embodiments, the LSD1 inhibitors may be administered in a local rather than systemic manner, such as by injection of the LSD1 inhibitor directly into a tissue, which is preferably subcutaneous or omental tissue, often in a depot or sustained release formulation. In other embodiments, the LSD1 inhibitors are systemically administered.

Furthermore, the LSD1 inhibitors may be administered in a targeted drug delivery system, such as in a particle which is suitable targeted to and taken up selectively by a cell or tissue. In some embodiments, the LSD1 inhibitor is contained or otherwise associated with a vehicle selected from liposomes, micelles, dendrimers, biodegradable particles, artificial DNA nanostructure, lipid-based nanoparticles and carbon or old nanoparticles. In illustrative examples of this type, the vehicle is selected from poly(lactic acid) (PLA), poly(glycolic acid) (PGA), poly(lactic-co-glycolic acid) (PLGA), poly(ethylene glycol) (PEG), PLA-PEG copolymers and combinations thereof.

In cases of local administration or selective uptake, the effective local concentration of the agent may not be related to plasma concentration.

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While the LSD1 inhibitor may be the sole active ingredient administered to the subject, the administration of other active ingredients concurrently with said LSD1 inhibitor is within the scope of the invention. For example, in some embodiments, the LSD1 inhibitor may be administered concurrently with one or more cancer therapies or anti-infective agents. The LSD1 inhibitor may be therapeutically used after the cancer therapy or anti-infective agent or may be therapeutically used together with the cancer therapy or anti-infective agent. The LSD1 inhibitor may be administered separately, simultaneously or sequentially with the other active ingredient.

Accordingly, in another aspect of the invention, there is provided a composition comprising an LSD1 inhibitor and an anti-infective agent. The present invention also contemplates a composition comprising an LSD1 inhibitor and a cancer therapy.

Suitable cancer therapies include, but are not limited to, radiotherapy, surgery, chemotherapy, hormone ablation therapy, pro-apoptosis therapy and immunotherapy.

Suitable radiotherapies include radiation and waves that induce DNA damage, for example, γ-irradiation, X-rays, UV irradiation, microwaves, electronic emissions and radioisotopes. Typically, therapy may be achieved by irradiating the localized tumor site with the above described forms of radiations. It is most likely that all of these factors cause a broad range of damage to DNA, on the precursors of DNA, on the replication and repair of DNA and on the assembly and maintenance of chromosomes.

The dosage range for X-rays ranges from daily doses of 50-200 roentgens for prolonged periods of time such as 3-4 weeks, to single doses of 2000-6000 roentgens. Dosage ranges for radioisotopes vary widely and depend on the half life of the isotope, the strength and type of radiation emitted and the uptake by the neoplastic cells. Suitable radiotherapies may include, but are not limited to, conformal external beam radiotherapy (50-100 Gray given as fractions over 4-8 weeks), either single shot or fractionated high dose brachytherapy, permanent interstitial brachytherapy and systemic radioisotopes such as Strontium 89. In some embodiments, the radiotherapy may be administered with a radiosensitizing agent. Suitable radiosensitizing agents may include, but are not limited to, efaproxiral, etanidazole, fluosol, misonidazole, nimorazole, temoporfin and tirapazamine.

Suitable chemotherapeutic agents may include, but are not limited to, antiproliferative/antineoplastic drugs and combinations thereof including alkylating agents (for example cisplatin, carboplatin, cyclophosphamide, nitrogen mustard, melphalan, chlorambucil, busulphan and nitrosoureas), antimetabolites (for example antifolates such as fluoropyridines like 5-fluorouracil and tegafur, raltitrexed, methotrexate, cytosine arabinoside and hydroxyurea), anti-tumor antibiotics (for example anthracyclines like adriamycin, bleomycin, doxorubicin, daunomycin, epirubicin, idarubicin, mitomycin-C, dactinomycin and mithramycin), antimitotic agents (for example Vinca alkaloids like vincristine, vinblastine, vindesine and vinorelbine and taxoids like paclitaxel and docetaxel), and topoisomerase inhibitors (for example epipodophyllotoxins like etoposide and teniposide, amsacrine, topotecan and camptothecin); cytostatic agents such as antiestrogens (for example tamoxifen, toremifene, raloxifene, droloxifene and idoxifene), estrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5α-reductase such as finasteride; agents which inhibit cancer cell invasion (for example metalloproteinase inhibitors like marimastat and inhibitors of urokinase plasminogen activator receptor function); inhibitors of growth factor function, for example such inhibitors include growth factor antibodies, growth factor receptor antibodies (for example the anti-erbb2 antibody trastuzumab [Herceptin™] and the anti-erbb1 antibody Cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (Gefitinib, AZD1839), N-(3-ethynylphenyl)-6,7-bis(2-methoxyethoxy)quinazolin-4-amine (Erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family; anti-angiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin); vascular damaging agents such as Combretastatin A4 and compounds disclosed in International Patent Applications WO 99/02166, WO 00/40529, WO 00/41669, WO 01/92224, WO 02/04434 and WO 02/08213; antisense therapies, for example those which are directed to the targets listed above, such as ISIS 2503, an anti-ras antisense; and gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Suitable immunotherapy approaches may include, but are not limited to ex vivo and in vivo approaches to increase the immunogenicity of patient tumor cells such as transfection with cytokines including interleukin 2, interleukin 4 or granulocyte-macrophage colony stimulating factor; approaches to decrease T-cell anergy; approaches using transfected immune cells such as cytokine-transfected dendritic cells; approaches using cytokine-transfected tumor cell lines; and approaches using anti-idiotypic antibodies. These approaches generally rely on the use of immune effector cells and molecules (which are encompassed herein as “immune-modulating agents”) to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

Examples of other cancer therapies include phytotherapy, cryotherapy, toxin therapy or pro-apoptosis therapy. A person skilled in the art would appreciate that this list is not exhaustive of the types of treatment modalities available for cancer and other hyperplastic lesions.

Suitable anti-infective agents include, but are not limited to, antimicrobials, which may include, but are not limited to, compounds that kill or inhibit the growth of microorganisms such as viruses, bacteria, yeast, fungi, protozoa, etc. and, thus, include antibiotics, amebicides, antifungals, antiprotozoals, antimalarials, antituberculotics and antivirals. Anti-infective drugs also include within their scope anthelmintics and nematocides.

Illustrative antibiotics include quinolones (e.g. amifloxacin, cinoxacin, ciprofloxacin, enoxacin, fleroxacin, flumequine, lomefloxacin, nalidixic acid, norfloxacin, ofloxacin, levofloxacin, oxolinic acid, pefloxacin, rosoxacin, temafloxacin, tosufloxacin, sparfloxacin, clinafloxacin, gatifloxacin, moxifloxacin, gemifloxacin, trovafloxacin and garenoxacin), tetracyclines, glycylcyclines and oxazolidinones (e.g. chlortetracycline, demeclocycline, doxycycline, lymecycline, methacycline, minocycline, oxytetracycline, tetracycline, tigecycline, linezolid, tedizolid and eperezolid), glycopeptides, aminoglycosides (e.g. amikacin, arbekacin, butirosin, dibekacin, fortimicins, gentamicin, kanamycin, menomycin, neomycin, netilmicin, paromomycin, ribostamycin, sisomicin, spectinomycin, streptomycin, tobramycin), β-lactams (e.g. imipenem, meropenem, biapenem, cefaclor, cefadroxil, cefamandole, cefatrizine, cefazedone, cefazolin, cefixime, cefmenoxime, cefodizime, cefonicid, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglycin, cephaloridine, cephalothin, cephapirin, cephradine, cefinetazole, cefoxitin, cefotetan, clavulanic acid, azthreonam, carumonam, flomoxef, moxalactam, amdinocillin, amoxicillin, ampicillin, azlocillin, carbenicillin, benzylpenicillin, carfecillin, cloxacillin, dicloxacillin, methicillin, mezlocillin, nafcillin, oxacillin, penicillin G, piperacillin, sulbenicillin, temocillin, ticarcillin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g. azithromycin, clarithromycin, erythromycin, fidaxomicin, oleandomycin, rokitamycin, rosaramicin, roxithromycin, troleandomycin), ketolides (e.g. telithromycin, cethromycin), coumermycins, lincosamides (e.g. clindamycin, lincomycin), chloramphenicol and salts and combinations thereof.

Illustrative antivirals include abacavir, acyclovir, adefovir, amantadine, amprenavir, atazanavir, boceprevir, cidofovir, daclatasvir, darunavir, delavirdine, didanosine, dolutegravir, efavirenz, elvitegravir, emtricitabine, enfuvirtide, entacavir, etravirine, famciclovir, fomivirsen, fosamprenavir, foscarnet, ganciclovir, indinavir, lamivudine, lamivudine/zidovudine, lopenavir, maraviroc, nelfinavir, nevirapine, oseltamivir, PEG-interferon alpha-2b, peramivir, raltegravir, ribavirin, rilpivirine, rimantadine, ritonavir, saquinavir, simeprevir, sofosbuvir, stavudine, telaprevir, telbivudine, tenofovir, tipranavir, valacyclovir, valganciclovir, zalcitabine, zanamivir, zidovudine and salts and combinations thereof.

Suitable amebicides or antiprotozoals include, but are not limited to, atovaquone, chloroquine, iodoquinol, mefloquine, metronidazole, nitazoxanide, paramomycin, pentamidine, tinidazole and salts and combinations thereof. Anthelmintics can be at least one selected from mebendazole, pyrantel, praziquantel, miltefosine, albendazole, ivermectin, thiabendazole and salts and combinations thereof. Illustrative antifungals can be selected from amphotericin B, amphotericin B cholesteryl sulfate complex, amphotericin B lipid complex, amphotericin B liposomal, anidulafungin, caspofungin, clotrimazole, fluconazole, flucytosine, griseofulvin, griseofulvin microsize, griseofulvin ultramicrosize, isavuconazonium, itraconazole, ketoconazole, micafungin, miconazole, nystatin, posaconazole, terbinafine, voriconazole and salts and combinations thereof. Suitable antimalarials include, but are not limited to, chloroquine, doxycycline, hydroxychloroquine, mefloquine, primaquine, pyrimethamine, pyrimethamine with sulfadoxine, quinine and salts and combinations thereof. Antituberculotics include but are not restricted to aminosalicylic acid, bedaquiline, capreomycin, clofazimine, cycloserine, dapsone, ethambutol, ethionamide, isoniazid, pyrazinamide, rifabutin, rifampin, rifapentine, streptomycin and salts and combinations thereof.

It is well known that chemotherapy and radiation therapy target rapidly dividing cells and/or disrupt the cell cycle or cell division. These treatments are offered as part of the treating several forms of cancer, aiming either at slowing their progression or reversing the symptoms of disease by means of a curative treatment. However, these cancer treatments may lead to an immunocompromised state and ensuing pathogenic infections and, thus, the present invention also extends to combination therapies, which employ an LSD1 inhibitor, a cancer therapy and an anti-infective agent that is effective against an infection that develops or that has an increased risk of developing from an immunocompromised condition resulting from the cancer therapy. Suitable anti-infective agents are as described above.

As previously described, the LSD1 inhibitor may be compounded for convenient and effective administration in effective amounts with a suitable pharmaceutically acceptable carrier in dosage unit form. In some embodiments, a unit dosage form may comprise the LSD1 inhibitor in an amount in the range of from about 0.25 μg to about 2000 mg. The LSD1 inhibitor may be present in an amount of from about 0.25 μg to about 2000 mg/mL of carrier. In embodiments where the pharmaceutical composition comprises one or more additional active ingredients, the dosages are determined by reference to the usual dose and manner of administration of the said ingredients.

### 4. Methods of Immune Checkpoint Inhibition

The present inventors have determined that inhibitors of LSD1 inhibit immune checkpoints, particularly PD-L1 and/or PD-L2, including the nuclear translocation of PD-L1 and/or PD-L2, especially PD-L1. Accordingly, the inventors have conceived that LSD1 inhibitors may be used for a range of applications, including for enhancing an immune response in a subject to a target antigen by an immune-modulating agent, for enhancing the efficacy of an anti-infective or for the treatment of a cancer such as a metastatic cancer or an infection.

Thus, in another aspect of the invention, there is provided a method of inhibiting PD-L1 and/or PD-L2 activity comprising contacting a PD-L1 and/or PD-L2 overexpressing cell with an LSD1 inhibitor. The present invention also contemplates an LSD1 inhibitor for use in inhibiting PD-L1 and/or PD-L2 activity.

The inhibition of PD-L1 and/or PD-L2 activity includes, but is not limited to, the inhibition of the interaction of PD-L1 and/or PD-L2 with PD-1, the expression of PD-L1 and/or PD-L2, or the nuclear translocation of PD-L1 and/or PD-L2.

Accordingly, in yet another aspect of the invention, there is provided a method of inhibiting the nuclear translocation of PD-L1 and/or PD-L2 in a PD-L1 and/or PD-L2 overexpressing cell, comprising contacting the PD-L1 and/or PD-L2 overexpressing cell with an LSD1 inhibitor. The present invention also provides a use of an LSD1 inhibitor for inhibiting the nuclear translocation of PD-L1 and/or PD-L2 in a PD-L1 and/or PD-L2 overexpressing cell; an LSD1 inhibitor for use in inhibiting the nuclear translocation of PD-L1 and/or PD-L2 in a PD-L1 and/or PD-L2 overexpressing cell; and the use of an LSD1 inhibitor in the manufacture of a medicament for inhibiting the nuclear translocation of PD-L1 and/or PD-L2 in a PD-L1 and/or PD-L2 overexpressing cell.

In particular embodiments, the PD-L1 and/or PD-L2 overexpressing cell is a cancer stem cell or a non-cancer stem cell tumor cell, especially a cancer stem cell tumor cell.

The present invention also contemplates an LSD1 inhibitor for use in inhibiting PD-L1 and/or PD-L2 activity in a subject, and the use of an LSD1 inhibitor in the manufacture of a medicament for inhibiting PD-L1 and/or PD-L2 activity in a subject. In some embodiments, the subject has elevated PD-L1 and/or PD-L2 activity. In some embodiments, the LSD1 inhibitor inhibits the nuclear translocation of PD-L1 and/or PD-L2 in a subject, especially PD-L1.

Accordingly, the present invention also extends to a method of inhibiting the nuclear translocation of PD-L1 and/or PD-L2 in subject, comprising administering an LSD1 inhibitor to the subject. In another aspect, the invention provides a use of an LSD1 inhibitor for inhibiting the nuclear translocation of PD-L1 and/or PD-L2 in a subject. The invention also provides a use of an LSD1 inhibitor in the manufacture of a medicament for inhibiting the nuclear translocation of PD-L1 and/or PD-L2 in a subject, and an LSD1 inhibitor for use in inhibiting the nuclear translocation of PD-L1 and/or PD-L2 in a subject.

In some embodiments, the LSD1 is a selective LSD1 inhibitor. In alternative embodiments, the LSD1 inhibitor is a non-selective LSD1 inhibitor.

There are several conditions involving PD-L1 and/or PD-L2 activity. Therefore, the present invention also contemplates the use of an LSD1 inhibitor for the treatment of a condition in which inhibition of PD-L1 and/or PD-L2 activity is associated with effective treatment, including increased efficacy of treatment. The present invention also provides a method for treating a condition in a subject in which inhibition of PD-L1 and/or PD-L2 is associated with effective treatment, comprising administering an LSD1 inhibitor to the subject. In a further aspect, the present invention provides the use of an LSD1 inhibitor in the manufacture of a medicament for treating a condition in a subject in which inhibition of PD-L1 and/or PD-L2 is associated with effective treatment.

Conditions involving PD-L1 and/or PD-L2 activity include, but are not limited to, a cancer particularly a metastatic cancer, or an infection, particularly a pathogenic infection.

Thus, in some embodiments, the subject has a cancer or an infection. In particular embodiments, the subject has a cancer, particularly a metastatic cancer.

In some embodiments, the cancer is selected from, but is not limited to, breast, prostate, lung, bladder, pancreatic, colon, melanoma, retinoblastoma, liver or brain cancer; especially breast cancer; most especially metastatic breast cancer.

In some embodiments, the subject has an infection, particularly a pathogenic infection. The infection may be selected from, but is not limited to, a viral, bacterial, yeast, fungal, helminth or protozoan infection. Viral infections contemplated by the present invention include, but are not restricted to, infections caused by HIV, hepatitis, influenza virus, Japanese encephalitis virus, Epstein-Barr virus, herpes simplex virus, filovirus, human papillomavirus, human T-cell lymphotropic virus, human retrovirus, cytomegalovirus, varicella-zoster virus, poliovirus, measles virus, rubella virus, mumps virus, adenovirus, enterovirus, rhinovirus, ebola virus, west nile virus and respiratory syncytial virus; especially infections caused by HIV, hepatitis, influenza virus, Japanese encephalitis virus, Epstein-Barr virus and respiratory syncytial virus. Bacterial infections include, but are not restricted to, those caused by Neisseria species, Meningococcal species, Haemophilus species, Salmonella species, Streptococcal species, Legionella species, Mycoplasma species, Bacillus species, Staphylococcus species, Chlamydia species, Actinomyces species, Anabaena species, Bacteroides species, Bdellovibrio species, Bordetella species, Borrelia species, Campylobacter species, Caulobacter species, Chlrorbium species, Chromatium species, Chlostridium species, Corynebacterium species, Cytophaga species, Deinococcus species, Escherichia species, Francisella species, Helicobacter species, Haemophilus species, Hyphomicrobium species, Leptospira species, Listeria species, Micrococcus species, Myxococcus species, Nitrobacter species, Oscillatoria species, Prochloron species, Proteus species, Pseudomonas species, Rhodospirillum species, Rickettsia species, Shigella species, Spirillum species, Spirochaeta species, Streptomyces species, Thiobacillus species, Treponema species, Vibrio species, Yersinia species, Nocardia species and Mycobacterium species; especially infections caused by Neisseria species, Meningococcal species, Haemophilus species, Salmonella species, Streptococcal species, Legionella species and Mycobacterium species. Protozoan infections encompassed by the invention include, but are not restricted to, those caused by Plasmodium species, Leishmania species, Trypanosoma species, Toxoplasma species, Entamoeba species and Giardia species. Helminth infections may include, but are not limited to, infections caused by Schistosoma species. Fungal infections contemplated by the present invention include, but are not limited to, infections caused by Histoplasma species and Candida species.

In particular embodiments, the LSD1 inhibitor is a proteinaceous molecule comprising, consisting or consisting essentially of a sequence corresponding to residues 108 to 118 of LSD1 as broadly described above; particularly a proteinaceous molecule comprising, consisting or consisting essentially of Formula I, SEQ ID NO: 1, 2 or 3, or a variant thereof.

The present invention also extends to the use of an LSD1 inhibitor for enhancing the efficacy of an anti-infective agent. Accordingly, in another aspect of the invention, there is provided a method of enhancing the efficacy of an anti-infective agent in a subject, comprising administering an LSD1 inhibitor to the subject. The invention also provides the use of an LSD1 inhibitor in the manufacture of a medicament for enhancing the efficacy of an anti-infective agent in a subject, and an LSD1 inhibitor for use in enhancing the efficacy of an anti-infective agent in a subject.

The anti-infective agent may include, but is not limited to, an antimicrobial, which may include, but is not limited to, any compound that kills or inhibit the growth of microorganisms such as viruses, bacteria, yeast, fungi, protozoa, etc. and, thus, includes antibiotics, amebicides, antifungals, antiprotozoals, antimalarials, antituberculotics and antivirals. Anti-infective agents also include within their scope anthelmintics and nematocides. Illustrative antibiotics, amebicides, antifungals, antiprotozoals, antimalarials, antituberculotics and antivirals are described in Section 3 supra.

In particular embodiments, the subject has an infection, especially a pathogenic infection. The infection may be selected from, but is not limited to, a viral, bacterial, yeast, fungal, helminth or protozoan infection as described supra.

Furthermore, the present inventors have conceived that an LSD1 inhibitor is useful for enhancing an immune response in a subject to a target antigen by an immune-modulating agent. Thus, the present invention also contemplates an LSD1 inhibitor for use in enhancing an immune response in a subject to a target antigen by an immune-modulating agent and the use of an LSD1 inhibitor in the manufacture of a medicament for enhancing an immune response in a subject to a target antigen by an immune-modulating agent.

The LSD1 inhibitor and the immune-modulating agent may be administered simultaneously, separately or sequentially. Accordingly, the present invention also extends to compositions comprising an LSD1 inhibitor and an immune-modulating agent. Such compositions can be administered to a subject either by themselves or in formulations where they are combined with a pharmaceutically acceptable carrier or diluent. Suitable formulations are described in Section 3.

In some embodiments, the immune-modulating agent includes, but is not limited to, an antigen that corresponds to at least a portion of the target antigen, an antigen binding molecule that is immuno-interactive with the target antigen and an immune modulating cell that modulates an immune response to the target antigen.

The antigen may be any antigen that corresponds to at least a portion of a target antigen of interest for stimulating an immune response to the target antigen. Such an antigen may be in soluble form (e.g. peptide, polypeptide or a nucleic acid molecule from which a peptide or polypeptide is expressible) or in the form of whole cells or attenuated pathogen preparations (e.g. attenuated virus or bacteria) or it may be presented by antigen-presenting cells as described in more detail below.

Target antigens useful in the present invention can be any type of biological molecule including, for example, simple intermediary metabolites, sugars, lipids, and hormones as well as macromolecules such as complex carbohydrates, phospholipids, nucleic acids, polypeptides and peptides. Target antigens may be selected from endogenous antigens produced by a host or exogenous antigens that are foreign to the host. Suitable endogenous antigens may include, but are not limited to, cancer or tumor antigens.

Non-limiting examples of cancer or tumor antigens include antigens from a cancer or tumor selected from ABL1 protooncogene, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous t-cell lymphoma, dermatofibrosarcoma-protuberans, desmoplastic-small-round-cell-tumor, ductal carcinoma, endocrine cancers, endometrial cancer, ependymoma, esophageal cancer, Ewing's Sarcoma, Extra-Hepatic Bile Duct Cancer, Eye Cancer, Eye: Melanoma, Retinoblastoma, Fallopian Tube cancer, Fanconi anaemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-Macroglobulinemia, Wilms' Tumor. Illustrative examples of tumor-specific antigens include, but are not limited to: etv6, aml1, cyclophilin b (acute lymphoblastic leukemia); Ig-idiotype (B cell lymphoma); E-cadherin, α-catenin, β-catenin, γ-catenin, p120ctn (glioma); p21ras (bladder cancer); p21ras (biliary cancer); MUC family, HER2/neu, c-erbB-2 (breast cancer); p53, p21ras (cervical carcinoma); p21ras, HER2/neu, c-erbB-2, MUC family, Cripto-1protein, Pim-1 protein (colon carcinoma); Colorectal associated antigen (CRC)-CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); melanocyte differentiation antigen (e.g. gp100, MART, Melan-A/MART-1, TRP-1, Tyros, TRP2, MC1R, MUC1F, MUC1R or a combination thereof) and melanoma-specific antigens (e.g. BAGE, GAGE-1, gp100In4, MAGE-1 (e.g. GenBank Accession No. X54156 and AA494311), MAGE-3, MAGE4, PRAME, TRP2IN2, NYNSO1a, NYNSO1b, LAGE1, p97 melanoma antigen (e.g. GenBank Accession No. M12154) p5 protein, gp75, oncofetal antigen, GM2 and GD2 gangliosides, cdc27, p21ras, gp100^{Pmel117} (melanoma); MUC family, p21ras (myeloma); HER2/neu, c-erbB-2 (non-small cell lung carcinoma); Imp-1, EBNA-1 (nasopharyngeal cancer); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papilloma virus proteins (squamous cell cancers of the cervix and oesophagus); NY-ESO-1 (testicular cancer); HTLV-1 epitopes (T cell leukemia); and combinations thereof.

Foreign antigens are suitably antigens from pathogenic organisms.

Exemplary pathogenic organisms include, but are not limited to, viruses, bacteria, fungi parasites, algae and protozoa and amoebae. Illustrative examples of viruses include viruses responsible for diseases including, but not limited to, measles, mumps, rubella, poliomyelitis, hepatitis A, B (e.g. GenBank Accession No. E02707), and C (e.g. GenBank Accession No. E06890), as well as other hepatitis viruses, influenza, adenovirus (e.g. types 4 and 7), rabies (e.g. GenBank Accession No. M34678), yellow fever, Epstein-Barr virus and other herpesviruses such as papillomavirus, Ebola virus, influenza virus, Japanese encephalitis (e.g. GenBank Accession No. E07883), dengue (e.g. GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g. GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g. hepatitis A, B, and C, viral components such as hepatitis C viral RNA. Illustrative examples of influenza viral antigens include, but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, refer to: Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, illustrative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g. Corynebacterium diphtheria), pertussis (e.g. Bordetella pertussis, GenBank Accession No. M35274), tetanus (e.g. Clostridium tetani, GenBank Accession No. M64353), tuberculosis (e.g. Mycobacterium tuberculosis), bacterial pneumonias (e.g. Haemophilus influenzae), cholera (e.g. Vibrio cholerae), anthrax (e.g. Bacillus anthracis), typhoid, plague, shigellosis (e.g. Shigella dysenteriae), botulism (e.g. Clostridium botulinum), salmonellosis (e.g. GenBank Accession No. L03833), peptic ulcers (e.g. Helicobacter pylori), Legionnaire's Disease, Lyme disease (e.g. GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to, pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen components. Also included with the bacterial antigens described herein are any other bacterial, mycobacterial, mycoplasmal, rickettsial or chlamydial antigens.

Illustrative examples of protozoa include protozoa that are responsible for diseases including, but not limited to, malaria (e.g. GenBank Accession No. X53832), hookworm, onchocerciasis (e.g. GenBank Accession No. M27807), schistosomiasis (e.g. GenBank Accession No. LOS 198), toxoplasmosis, trypanosomiasis, leishmaniasis, giardiasis (GenBank Accession No. M33641), amoebiasis, filariasis (e.g. GenBank Accession No. J03266), borreliosis and trichinosis. Thus, protozoal antigens which can be used in the compositions and methods of the invention include, but are not limited to, Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components.

The present invention also contemplates toxin components as antigens. Illustrative examples of toxins include, but are not restricted to, staphylococcal enterotoxins, toxic shock syndrome toxin, retroviral antigens (e.g. antigens derived from HIV), streptococcal antigens, staphylococcal enterotoxin-A (SEA), staphylococcal enterotoxin-B (SEB), staphylococcal enterotoxinₗ₋₃ (SE₁₋₃), staphylococcal enterotoxin-D (SED), staphylococcal enterotoxin-E (SEE) as well as toxins derived from Mycoplasma, Mycobacterium and herpes viruses.

The antigen corresponding to at least a portion of the target antigen may be isolated from a natural source or may be prepared by recombinant techniques as known in the art. For example, peptide antigens can be eluted from the MHC and other presenting molecules of antigen-presenting cells obtained from a cell population or tissue for which a modified immune response is desired. The eluted peptides can be purified using standard protein purification techniques known in the art (Rawson et al. (2000), Cancer Res, 60(16): 4493-4498). If desired, the purified peptides can be sequenced and synthetic versions of the peptides produced using standard protein synthesis techniques as for example described below. Alternatively, crude antigen preparations can be produced by isolating a sample of a cell population or tissue for which a modified immune response is desired, and either lysing the sample or subjecting the sample to conditions that will lead to the formation of apoptotic cells (e.g. irradiation with ultra violet or with γ rays, viral infection, cytokines or by depriving cells of nutrients in the cell culture medium, incubation with hydrogen peroxide, or with drugs such as dexamethasone, ceramide chemotherapeutics and anti-hormonal agents such as Lupron or Tamoxifen). The lysate or the apoptotic cells can then be used as a source of crude antigen for use in soluble form or for contact with antigen-presenting cells as described in more detail below.

When the antigen is known, it may be conveniently prepared in recombinant form using standard protocols as for example described in: Sambrook et al. (1989) Molecular Cloning: A Laboratory manual (Cold Spring Harbor Press), in particular Sections 16 and 17; Ausubel et al. (1998), Current Protocols in Molecular Biology (John Wiley & Sons, Inc.), in particular Chapters 10 and 16; and Coligan et al. (1997), Current Protocols in Protein Science (John Wiley & Sons, Inc.), in particular Chapters 1, 5 and 6. Typically, an antigen may be prepared by a procedure including the steps of (a) providing an expression vector from which the target antigen or analogue or mimetic thereof is expressible; (b) introducing the vector into a suitable host cell; (c) culturing the host cell to express recombinant polypeptide from the vector; and (d) isolating the recombinant polypeptide.

In general, the expression vector will comprise an antigen-encoding polynucleotide which is operably connected to a regulatory polynucleotide. The antigen-encoding polynucleotide can be constructed from any suitable parent polynucleotide that codes for an antigen that corresponds to the target antigen of interest. The parent polynucleotide is suitably a natural gene or portion thereof. However, it is possible that the parent polynucleotide is not naturally-occurring but has been engineered using recombinant techniques. The regulatory polynucleotide suitably comprises transcriptional and/or translational control sequences, which will generally be appropriate for the host cell used for expression of the antigen-encoding polynucleotide. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the host cell to be introduced or may be derived from an alternative source, where the region is functional in the host cell.

The expression vector may also comprise a 3′ non-translated sequence, which usually refers to that portion of a gene comprising a DNA segment that contains a polyadenylation signal and any other regulatory signals capable of effecting mRNA processing or gene expression. The polyadenylation signal is characterised by effecting the addition of polyadenylic acid tracts to the 3′ end of the mRNA precursor. Polyadenylation signals are commonly recognised by the presence of homology to the canonical form 5′ AATAAA-3′ although variations are not uncommon. The 3′ non-translated regulatory DNA sequence typically includes from about 50 to 1,000 nucleotide base pairs and may contain transcriptional and translational termination sequences in addition to a polyadenylation signal and any other regulatory signals capable of effecting mRNA processing or gene expression.

In certain embodiments, the expression vector further contains a selectable marker gene to allow the selection of transformed host cells. Selection genes are well known in the art and will vary with the host cell used.

The expression vector may also include a fusion partner (typically provided by the expression vector) so that the recombinant polypeptide is expressed as a fusion polypeptide with the fusion partner. The main advantage of fusion partners is that they assist identification and/or purification of said fusion polypeptide. Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners also include within their scope “epitope tags”, which are usually short peptide sequences for which a specific antibody is available. Well known examples of epitope tags for which specific monoclonal antibodies are readily available include c-Myc, influenza virus haemagglutinin and FLAG tags.

The step of introducing the expression vector into the host cell may be achieved by any suitable method including transfection, transduction of viral vectors, including adenoviral, modified lentiviral and other retroviral vectors, and transformation, the choice of which will be dependent on the host cell employed. Such methods are well known to those skilled in the art.

Recombinant polypeptides may be produced by culturing a host cell transformed with the expression vector under conditions appropriate for protein expression, which will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilised with a baculovirus expression system.

In some embodiments, the antigen, which may be administered with the LSD1 inhibitor, is in the form of a construct or vector from which it is expressible.

Alternatively, the antigen can be synthesised using solution synthesis or solid phase synthesis as described, for example, by Atherton and Sheppard (1989, Solid Phase Peptide Synthesis: A Practical Approach, IRL Press at Oxford University Press, Oxford, England) or by Roberge et al. (1995, Science, 269: 202). The amino acids of the synthesised antigens can be non-naturally occurring or naturally occurring amino acids. Examples of unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in Table 3 (supra).

The invention also contemplates modifying peptide antigens using ordinary molecular biological techniques so as to alter their resistance to proteolytic degradation or to optimise solubility properties or to render them more suitable as an immunogenic agent.

Peptide antigens may be of any suitable size that can be utilized to stimulate or inhibit an immune response to a target antigen of interest. A number of factors can influence the choice of peptide size. For example, the size of a peptide can be chosen such that it includes, or corresponds to the size of, T cell epitopes and/or B cell epitopes, and their processing requirements. Practitioners in the art will recognise that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilising binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class II-restricted epitopes, are quite variable although such epitopes are frequently smaller in size than 15 amino acid residues. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 500, 200, 100, 80, 60, 50, 40 amino acid residues. In certain advantageous embodiments, the size of the peptide is sufficient for presentation by an antigen-presenting cell of a T cell and/or a B cell epitope contained within the peptide.

Criteria for identifying and selecting effective antigenic peptides (e.g. minimal peptide sequences capable of eliciting an immune response) can be found in the art. For example, Apostolopoulos et al. (2000, Curr. Opin. Mol. Ther., 2: 29-36) discusses the strategy for identifying minimal antigenic peptide sequences based on an understanding of the three dimensional structure of an antigen-presenting molecule and its interaction with both an antigenic peptide and T-cell receptor. Shastri (1996, Curr. Opin. Immunol., 8: 271-277) discloses how to distinguish rare peptides that serve to activate T cells from the thousands peptides normally bound to MHC molecules.

In some embodiments, the immune-modulating cell is an antigen-presenting cell, which presents an antigen corresponding to at least a portion of the target antigen. Such antigen-presenting cells include professional or facultative antigen-presenting cells. Professional antigen-presenting cells function physiologically to present antigen in a form that is recognised by specific T cell receptors so as to stimulate or anergise a T lymphocyte or B lymphocyte mediated immune response. Professional antigen-presenting cells not only process and present antigens in the context of the major histocompatability complex (MHC), but also possess the additional immunoregulatory molecules required to complete T cell activation or induce a tolerogenic response. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Non-professional or facultative antigen-presenting cells typically lack one or more of the immunoregulatory molecules required to complete T lymphocyte activation or anergy. Examples of non-professional or facultative antigen-presenting cells include, but are not limited to, activated T lymphocytes, eosinophils, keratinocytes, astrocytes, follicular cells, microglial cells, thymic cortical cells, endothelial cells, Schwann cells, retinal pigment epithelial cells, myoblasts, vascular smooth muscle cells, chondrocytes, enterocytes, thymocytes, kidney tubule cells and fibroblasts. In some embodiments, the antigen-presenting cell is selected from monocytes, macrophages, B lymphocytes, cells of myeloid lineage, dendritic cells or Langerhans cells. In certain advantageous embodiments, the antigen-presenting cell expresses CD11c and includes a dendritic cell.

In some embodiments the antigen-presenting cell stimulates an immune response, including a pro-inflammatory immune response.

Antigen-presenting cells for stimulating an immune response to an antigen or group of antigens to an antigen or group of antigens may be prepared according to any suitable method known to the skilled practitioner. Illustrative methods for preparing antigen-presenting cells for stimulating antigen-specific immune responses are described by Albert et al. (International Publication WO 99/42564), Takamizawa et al. (1997, J Immunol, 158(5): 2134-2142), Thomas and Lipsky (1994, J Immunol, 153(9): 4016-4028), O'Doherty et al. (1994, Immunology, 82(3): 487-93), Fearnley et al. (1997, Blood, 89(10): 3708-3716), Weissman et al. (1995, Proc Natl Acad Sci USA, 92(3): 826-830), Freudenthal and Steinman (1990, Proc Natl Acad Sci USA, 87(19): 7698-7702), Romani et al. (1996, J Immunol Methods, 196(2): 137-151), Reddy et al. (1997, Blood, 90(9): 3640-3646), Thurnher et al. (1997, Exp Hematol, 25(3): 232-237), Caux et al. (1996, J Exp Med, 184(2): 695-706; 1996, Blood, 87(6): 2376-85), Luft et al. (1998, Exp Hematol, 26(6): 489-500; 1998, J Immunol, 161(4): 1947-1953), Cella et al. (1999, J Exp Med, 189(5): 821-829; 1997, Nature, 388(644): 782-787; 1996, J Exp Med, 184(2): 747-572), Ahonen et al. (1999, Cell Immunol, 197(1): 62-72) and Piemonti et al. (1999, J Immunol, 162(11): 6473-6481).

In some embodiments, the antigen-presenting cells are isolated from a host, treated and then re-introduced or reinfused into the host. Conveniently, antigen-presenting cells can be obtained from the host to be treated either by surgical resection, biopsy, blood sampling, or other suitable technique. Such cells are referred to herein as “autologous” cells. In other embodiments, the antigen-presenting cells or cell lines are prepared and/or cultured from a different source than the host. Such cells are referred to herein as “allogeneic” cells. Desirably, allogeneic antigen-presenting cells or cell lines will share major and/or minor histocompatibility antigens to potential recipients (also referred to herein as “generic” antigen-presenting cells or cell lines). In certain advantageous embodiments of this type, the generic antigen-presenting cells or cell lines comprise major histocompatibility (MHC) class I antigens compatible with a high percentage of the population (i.e. at least 10, 20, 30, 40, 50, 60, 70, 75, 80, 85, 90, 92, 94 or 98%) that is susceptible or predisposed to a particular condition. Suitably, the generic antigen-presenting cells or cell lines naturally express an immunostimulatory molecule as described herein, especially an immunostimulatory membrane molecule, at levels sufficient to trigger an immune response, desirably a T lymphocyte immune response (e.g. a cytotoxic T lymphocyte immune response), in the intended host. In certain embodiments, the antigen-presenting cells or cell lines are highly susceptible to treatment with at least one IFN as described herein and in International Publication No. WO 01/88097 (i.e. implied high level expression of class I HLA).

In some embodiments, antigen-presenting cells are made antigen-specific by a process including contacting or ‘pulsing’ the antigen-presenting cells with an antigen that corresponds to at least a portion of the target antigen for a time and under conditions sufficient to permit the antigen to be internalised by the antigen-presenting cells; and culturing the antigen-presenting cells so contacted for a time and under conditions sufficient for the antigen to be processed for presentation by the antigen-presenting cells. The pulsed cells can then be used to stimulate autologous or allogeneic T cells in vitro or in vivo. The amount of antigen to be placed in contact with antigen-presenting cells can be determined empirically by a person of skill in the art. Typically, antigen-presenting cells are incubated with antigen for about 1 to 6 hr at 37° C. Usually, for purified antigens and peptides, 0.1-10 μg/mL is suitable for producing antigen-specific antigen-presenting cells. The antigen should be exposed to the antigen-presenting cells for a period of time sufficient for those cells to internalise the antigen. The time and dose of antigen necessary for the cells to internalise and present the processed antigen may be determined using pulse-chase protocols in which exposure to antigen is followed by a washout period and exposure to a read-out system e.g. antigen reactive T cells. Once the optimal time and dose necessary for cells to express processed antigen on their surface is determined, a protocol may be used to prepare cells and antigen for inducing tolerogenic responses. Those of skill in the art will recognise in this regard that the length of time necessary for an antigen-presenting cell to present an antigen may vary depending on the antigen or form of antigen employed, its dose, and the antigen-presenting cell employed, as well as the conditions under which antigen loading is undertaken. These parameters can be determined by the skilled artisan using routine procedures.

The delivery of exogenous antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang (1994), Immunomethods, 4: 229-235), osmotic lysis of pinosomes after pinocytic uptake of soluble antigen (Moore et al. (1988), Cell, 54: 777-785), coupling of antigens to potent adjuvants (Aichele et al. (1990), J. Exp. Med., 171: 1815-1820; Gao et al. (1991), J. Immunol., 147: 3268-3273; Schulz et al. (1991), Proc. Natl. Acad. Sci. USA, 88: 991-993; Kuzu et al. (1993), Euro. J. Immunol., 23: 1397-1400; and Jondal et al. (1996), Immunity, 5: 295-302) and apoptotic cell delivery of antigen (Albert et al. (1998), Nature, 392: 86-89; Albert et al. (1998), Nature Med., 4: 1321-1324; and in International Publications WO 99/42564 and WO 01/85207). Recombinant bacteria (e.g. E. coli) or transfected host mammalian cells may be pulsed onto dendritic cells (as particulate antigen, or apoptotic bodies respectively) for antigen delivery. Recombinant chimeric virus-like particles (VLPs) have also been used as vehicles for delivery of exogenous heterologous antigen to the MHC class I processing pathway of a dendritic cell line (Bachmann et al. (1996), Eur. J. Immunol., 26(11): 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see, e.g., Cox and Coulter (1997), Vaccine 15(3): 248-256 and U.S. Pat. No. 6,352,697), phospholipases (see, e.g., Camilli et al. (1991), J. Exp. Med., 173: 751-754), pore-forming toxins (e.g. an α-toxin), natural cytolysins of gram-positive bacteria, such as listeriolysin O (LLO; e.g. Mengaud et al. (1988), Infect. Immun., 56: 766-772; and Portnoy et al. (1992), Infect. Immun., 60: 2710-2717), streptolysin O (SLO; e.g. Palmer et al. (1998), Biochemistry 37(8): 2378-2383) and perfringolysin O (PFO; e.g. Rossjohn et al. (1997), Cell, 89(5): 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al. (1992), Infect. Immun., 60: 2710-2717).

The cytolysin may be provided together with a pre-selected antigen in the form of a single composition or may be provided as a separate composition, for contacting the antigen-presenting cells. In one embodiment, the cytolysin is fused or otherwise linked to the antigen, wherein the fusion or linkage permits the delivery of the antigen to the cytosol of the target cell. In another embodiment, the cytolysin and antigen are provided in the form of a delivery vehicle such as, but not limited to, a liposome or a microbial delivery vehicle selected from virus, bacterium, or yeast. Suitably, when the delivery vehicle is a microbial delivery vehicle, the delivery vehicle is non-virulent. In a preferred embodiment of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory polynucleotide which expresses the cytolysin in the bacterium, and a second polynucleotide encoding one or more pre-selected antigens. Non-secreted cytolysins may be provided by various mechanisms, e.g., absence of a functional signal sequence, a secretion incompetent microbe, such as microbes having genetic lesions (e.g. a functional signal sequence mutation), or poisoned microbes, etc. A wide variety of nonvirulent, non-pathogenic bacteria may be used; preferred microbes are relatively well characterised strains, particularly laboratory strains of E. coli, such as MC4100, MC1061, DH5α, etc. Other bacteria that can be engineered for the invention include well-characterized, nonvirulent, non-pathogenic strains of Listeria monocytogenes, Shigella flexneri, mycobacterium, Salmonella, Bacillus subtilis, etc. In a particular embodiment, the bacteria are attenuated to be non-replicative, non-integrative into the host cell genome, and/or non-motile inter- or intra-cellularly.

The delivery vehicles described above can be used to deliver one or more antigens to virtually any antigen-presenting cell capable of endocytosis of the subject vehicle, including phagocytic and non-phagocytic antigen-presenting cells. In embodiments when the delivery vehicle is a microbe, the subject methods generally require microbial uptake by the target cell and subsequent lysis within the antigen-presenting cell vacuole (including phagosomes and endosomes).

In other embodiments, the antigen is produced inside the antigen-presenting cell by introduction of a suitable expression vector as, for example, described above. The antigen-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimised polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5, 10, 15, 20, 25, 30%, more preferably 35, 40, 50, 60, 70% or more of the existing codons of a parent polynucleotide.

The expression vector for introduction into the antigen-presenting cell will be compatible therewith such that the antigen-encoding polynucleotide is expressible by the cell. For example, expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol. 11(2): 205-208), Vigna and Naldini (2000, J. Gene Med., 2(5): 308-316), Kay et al. (2001, Nat. Med., 7(1): 33-40), Athanasopoulos, et al. (2000, Int. J. Mol. Med., 6(4):363-375) and Walther and Stein (2000, Drugs, 60(2): 249-271). The expression vector is introduced into the antigen-presenting cell by any suitable means which will be dependent on the particular choice of expression vector and antigen-presenting cell employed. Such means of introduction are well-known to those skilled in the art. For example, introduction can be effected by use of contacting (e.g. in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g. liposomes. Such liposomes are commercially available (e.g. Lipofectin®, Lipofectamine™, and the like, supplied by Invitrogen, Waltham Mass., USA). It will be understood by persons of skill in the art that the techniques for assembling and expressing antigen-encoding nucleic acid molecules, immunoregulatory molecules and/or cytokines as described herein e.g. synthesis of oligonucleotides, nucleic acid amplification techniques, transforming cells, constructing vectors, expressions system and the like and transducing or otherwise introducing nucleic acid molecules into cells are well established in the art, and most practitioners are familiar with the standard resource materials for specific conditions and procedures.

In some embodiments, the antigen-specific antigen-presenting cells are obtained by isolating antigen-presenting cells or their precursors from a cell population or tissue to which modification of an immune response is desired. Typically, some of the isolated antigen-presenting cells or precursors will constitutively present antigens or have taken up such antigen in vivo that are targets or potential targets of an immune response for which stimulation or inhibition of an immune response is desired. In this instance, the delivery of exogenous antigen is not essential. Alternatively, cells may be derived from biopsies of healthy or diseased tissues, lysed or rendered apoptotic and then pulsed onto antigen-presenting cells (e.g. dendritic cells). In certain embodiments of this type, the antigen-presenting cells are cancer or tumor cells to which an antigen-specific immune response is required. Illustrative examples of cancers or tumor cells include cells of sarcomas and carcinomas e.g. fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; myelomonocytic, monocytic and erythroleukemia; chronic leukemia (chronic myelocytic (granulocyte) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In certain embodiments, the cancer or tumor cells are breast cancer cells.

In some of the above embodiments, the cancer or tumor cells will constitute facultative or non-professional antigen-presenting cells, and may in some instances require further modification to enhance their antigen-presenting functions. In these instances, the antigen-presenting cells are further modified to express one or more immunoregulatory molecules, which include any molecules occurring naturally in animals that may regulate or directly influence immune responses including: proteins involved in antigen processing and presentation such as TAP1/TAP2 transporter proteins, proteosome molecules such as LMP2 and LMP7, heat shock proteins such as gp96, HSP70 and HSP90, and major histocompatibility complex (MHC) or human leucocyte antigen (HLA) molecules; factors that provide co-stimulation signals for T cell activation such as B7 and CD40; factors that provide co-inhibitory signals for direct killing of T cells or induction of T lymphocyte or B lymphocyte anergy or stimulation of T regulatory cell (Treg) generation such as OX-2, PD-L1 or PD-L2; accessory molecules such as CD83; chemokines; lymphokines and cytokines such as interferons α, β and γ, interleukins (e.g., IL-2, IL-7, IL-12, IL-15, IL-22, etc.), factors stimulating cell growth (e.g. GM-SCF) and other factors (e.g. tumor necrosis factors (TNFs), DC-SIGN, MIP1a, MIP1P and transforming growth factor-β (TGF-β). In certain advantageous embodiments, the immunoregulatory molecules are selected from a B7 molecule (e.g. B7-1, B7-2 or B7-3) and an ICAM molecule (e.g. ICAM-1 and ICAM-2).

Instead of recombinantly expressing immunoregulatory molecules, antigen-presenting cells expressing the desired immunostimulatory molecule(s) may be isolated or selected from a heterogeneous population of cells. Any method of isolation/selection is contemplated by the present invention, examples of which are known to those of skill in the art. For instance, one can take advantage of one or more particular characteristics of a cell to specifically isolate that cell from a heterogeneous population. Such characteristics include, but are not limited to, anatomical location of a cell, cell density, cell size, cell morphology, cellular metabolic activity, cell uptake of ions such as Ca²⁺, K⁺, and H⁺ ions, cell uptake of compounds such as stains, markers expressed on the cell surface, protein fluorescence and membrane potential. Suitable methods that can be used in this regard include surgical removal of tissue, flow cytometry techniques such as fluorescence-activated cell sorting (FACS), immunoaffinity separation (e.g. magnetic bead separation such as Dynabead™ separation), density separation (e.g. metrizamide, Percoll™, or Ficoll™ gradient centrifugation), and cell-type specific density separation. Desirably, the cells are isolated by flow cytometry or by immunoaffinity separation using an antigen-binding molecule that is immuno-interactive with the immunoregulatory molecule.

Alternatively, the immunoregulatory molecule can be provided to the antigen-presenting cells in soluble form. In some embodiments of this type, the immunoregulatory molecule is a B7 molecule that lacks a functional transmembrane domain (e.g. that comprises a B7 extracellular domain), non-limiting examples of which are described by McHugh et al. (1998, Clin. Immunol. Immunopathol., 87(1): 50-59), Faas et al. (2000, J. Immunol., 164(12): 6340-6348) and Jeannin et al. (2000, Immunity, 13(3): 303-312). In other embodiments of this type, the immunostimulatory protein is a B7 derivative including, but not limited to, a chimeric or fusion protein comprising a B7 molecule, or biologically active fragment thereof, or variant or derivative of these, linked together with an antigen binding molecule such as an immunoglobulin molecule or biologically active fragment thereof. For example, a polynucleotide encoding the amino acid sequence corresponding to the extracellular domain of the B7-1 molecule, containing amino acids from about position 1 to about position 215, is joined to a polynucleotide encoding the amino acid sequences corresponding to the hinge, CH2 and CH3 regions of human Ig Cy1, using PCR, to form a construct that is expressed as a B7Ig fusion protein. DNA encoding the amino acid sequence corresponding to a B7Ig fusion protein has been deposited with the American Type Culture Collection (ATCC) in Rockville, Md., under the Budapest Treaty on May 31, 1991 and accorded accession number 68627. Techniques for making and assembling such B7 derivatives are disclosed for example by Linsley et al. (U.S. Pat. No. 5,580,756). Reference also may be made to Sturmhoefel et al. (1999, Cancer Res., 59: 4964-4972) who disclose fusion proteins comprising the extracellular region of B7-1 or B7-2 fused in frame to the Fc portion of IgG2a.

The half-life of a soluble immunoregulatory molecule may be prolonged by any suitable procedure if desired. Preferably, such molecules are chemically modified with polyethylene glycol (PEG), including monomethoxy-polyethylene glycol, as for example disclosed by Chapman et al (1999, Nature Biotechnology, 17: 780-783).

Alternatively, or in addition, the antigen-presenting cells are cultured in the presence of at least one interferon for a time and under conditions sufficient to enhance the antigen presenting function of the cells, and washing the cells to remove the interferon(s). In certain advantageous embodiments of this type, the step of culturing may comprise contacting the cells with at least one type I interferon and/or a type II interferon. The at least one type I interferon is suitably selected from the group consisting of an IFN-α, an IFN-β, a biologically active fragment of an IFN-α, a biologically active fragment of an IFN-β, a variant of an IFN-α, a variant of an IFN-β, a variant of a said biologically active fragment, a derivative of an IFN-α, a derivative of an IFN-β, a derivative of a said biologically active fragment, a derivative of a said variant, an analogue of IFN-α and an analogue of IFN-β. Typically, the type II interferon is selected from the group consisting of an IFN-γ, a biologically active fragment of an IFN-γ, a variant of an IFN-γ, a variant of said biologically active fragment, a derivative of an IFN-γ, a derivative of said biologically active fragment, a derivative of said variant and an analogue of an IFN-γ. Exemplary methods and conditions for enhancing the antigen-presenting functions of antigen-presenting cells using interferon treatment are described in International Publication No. WO 01/88097.

In some embodiments, the antigen-presenting cells (e.g. cancer cells) or cell lines are suitably rendered inactive to prevent further proliferation once administered to the subject. Any physical, chemical, or biological means of inactivation may be used, including but not limited to irradiation (generally with at least about 5,000 cGy, usually at least about 10,000 cGy, typically at least about 20,000 cGy); or treatment with mitomycin-C (usually at least 10 μg/mL; more usually at least about 50 μg/mL).

The antigen-presenting cells may be obtained or prepared to contain and/or express one or more antigens by any number of means, such that the antigen(s) or processed form(s) thereof, is (are) presented by those cells for potential modulation of other immune cells, including T lymphocytes and B lymphocytes, and particularly for producing T lymphocytes and B lymphocytes that are primed to respond to a specified antigen or group of antigens.

In some embodiments, the antigen-presenting cell is an immune-effector cell. Accordingly, in some embodiments, the antigen-presenting cells described above are useful for producing primed T lymphocytes to an antigen or group of antigens. In other embodiments, the antigen-specific antigen-presenting cells are useful for producing T lymphocytes that exhibit tolerance/anergy to an antigen or group of antigens. The efficiency of inducing lymphocytes, especially T lymphocytes, to exhibit an immune response or tolerance/anergy to a specified antigen can be determined by any suitable method including, but not limited to, assaying T lymphocyte cytolytic activity in vitro using for example antigen-specific antigen-presenting cells as targets of antigen-specific cytolytic T lymphocytes (CTL); assaying antigen-specific T lymphocyte proliferation (see, e.g., Vollenweider and Groseurth (1992), J. Immunol. Meth., 149: 133-135), measuring B cell response to the antigen using, for example, Elispot assays, and ELISA assays; interrogating cytokine profiles; or measuring delayed-type hypersensitivity (DTH) responses by test of skin reactivity to a specified antigen (see, e.g., Chang et al. (1993), Cancer Res. 53: 1043-1050). Other methods known to practitioners in the art, which can detect the presence of antigen on the surface of antigen-presenting cells after exposure to the antigen, are also contemplated by the present invention.

Accordingly, the present invention also provides antigen-specific B or T lymphocytes, especially T lymphocytes, which respond in an antigen-specific fashion to representation of the antigen. In some embodiments, antigen-specific T lymphocytes are produced by contacting an antigen-presenting cell as defined above with a population of T lymphocytes, which may be obtained from any suitable source such as spleen or tonsil/lymph nodes but is preferably obtained from peripheral blood. The T lymphocytes can be used as crude preparations or as partially purified or substantially purified preparations, which are suitably obtained using standard techniques as, for example, described in “Immunochemical Techniques, Part G: Separation and Characterization of Lymphoid Cells” (1984, Meth. in Enzymol. 108, Edited by Di Sabato et al., Academic Press). This includes rosetting with sheep red blood cells, passage across columns of nylon wool or plastic adherence to deplete adherent cells, immunomagnetic or flow cytometric selection using appropriate monoclonal antibodies known in the art.

The preparation of T lymphocytes is contacted with antigen-specific antigen-presenting cells as described herein for an adequate period of time for priming or anergising the T lymphocytes to the antigen or antigens presented by those antigen-presenting cells. This period will usually be at least about 1 day, and up to about 5 days.

In embodiments employing tolerance or anergy inducing antigen-specific antigen-presenting cells, the antigen-specific anergy induced by such cells desirably involves the induction of one or more types of antigen-specific regulatory lymphocytes, especially regulatory T lymphocytes. Several populations of regulatory T lymphocytes are known to inhibit the response of other (effector) lymphocytes in an antigen-specific manner including, for example, Tr1 lymphocytes, Th3 lymphocytes, Th2 lymphocytes, CD8⁺CD28⁻ regulatory T lymphocytes, CD4⁺CD25⁺ regulatory T lymphocytes, natural killer (NK) T lymphocytes and γδ T lymphocytes.

Tr1 lymphocytes can emerge after several rounds of stimulation of human blood T cells by allogeneic monocytes in the presence of IL-10. This subpopulation secretes high levels of IL-10 and moderate levels of TGFβ but little IL-4 or IFNγ (Groux et al. (1997), Nature, 389: 737-742).

The Th3 regulatory subpopulation refers to a specific subset induced following antigen delivery via the oral (or other mucosal) route. They produce predominantly TGFβ, and only low levels of IL-10, IL-4 or IFNγ, and provide specific help for IgA production (Weiner et al. (2001), Microbes Infect, 3: 947-954). They are able to suppress both Th1 and Th2-type effector T cells.

Th2 lymphocytes produce high levels of IL-4, IL-5 and IL-10 but low IFNγ and TGFβ. Th2 lymphocytes are generated in response to a relative abundance of IL-4 and lack of IL-12 in the environment at the time of presentation of their cognate peptide ligands (O'Garra and Arai (2000), Trends Cell Biol, 10: 542-550). T lymphocyte signalling by CD86 may also be important for generation of Th2 cells (Lenschow et al. (1996), Immunity, 5:285-293; Xu et al. (1997), J Immunol, 159: 4217-4226).

A distinct CD8⁺CD28⁻ regulatory or “suppressor” subset of T lymphocytes can be induced by repetitive antigenic stimulation in vitro. They are MHC class I-restricted, and suppress CD4+ T cell responses.

CD4⁺CD25⁺ regulatory or “suppressor” subset of T lymphocytes inhibit a variety of autoimmune and inflammatory diseases and they are also efficient in the suppression of alloantigen responses. In particular, these lymphocytes can down-regulate the immune response by affecting T cell responses, antibody production, cytokine secretion and antigen-presenting cells (see, e.g., Suvas et al. (2003), J Exp Med., 198(6): 889-901; Taams et al. (2003), Transpl Immunol., 11(3-4): 277-85; Jonuleit et al. (2003), Transpl Immunol., 11(3-4): 267-76; Green et al. (2003), Proc Natl Acad Sci USA., 100(19): 10878-10883). CD4⁺CD25⁺ regulatory T cells are generated by repetitive antigenic stimulation in vitro (Feunou et al. (2003), J Immunol., 171(7): 3475-84).

NK T lymphocytes, which express the NK cell marker, CD161, and whose TCR are Va24JαQ in human and Va14Jα281 in mouse, are activated specifically by the non-polymorphic CD1d molecule through presentation of a glycolipid antigen (Kawano et al. (1997), Science, 278: 1626-1629). They have been shown to be immunoregulatory in a number of experimental systems. They are reduced in number in several autoimmune models before disease onset, and can reduce incidence of disease upon passive transfer to non-obese diabetic (NOD) mice. Administration of the glycolipid, α-galactosyl ceramide (α-gal cer), presented by CD1d, also results in accumulation of NKT lymphocytes and amelioration of diabetes in these mice (Naumov et al. (2001), Proc Natl Acad Sci USA, 98: 13838-13843).

γδ T lymphocytes have been implicated in the downregulation of immune responses in various inflammatory diseases and in the suppression of inflammation associated with induction of mucosal tolerance. The tolerance induced by mucosal antigen was transferable to untreated recipient mice by small numbers of γδ T cells (McMenamin et al. (1995), J Immunol, 154: 4390-4394; McMenamin et al. (1994), Science, 265: 1869-1871). Moreover, mucosal tolerance induction was blocked by the administration of the GL3 antibody that blocks γδ T cell function (Ke et al. (1997), J Immunol, 158: 3610-3618).

Whether the antigen-specific T lymphocytes are produced in contact with antigen-presenting cells in vitro or in vivo, the antigen-specific anergy induced by the antigen-presenting cells reflects the inability of the antigen-specific lymphocytes to respond to subsequent restimulation with the specific antigen. These antigen-specific lymphocytes are suitably characterized by production of IL-10 in an antigen-specific manner. IL-10 is a cytokine with potent immunosuppressive properties. IL-10 inhibits antigen-specific T lymphocyte proliferation at different levels. IL-10 inhibits the antigen-presenting and accessory cell function of professional antigen-presenting cells such as monocytes, dendritic cells and Langerhans cells by downregulation of the expression of MHC class II molecules and of the adhesion and co-stimulatory molecules ICAM-1 and B7.1 and B7.2 (reviewed in Interleukin 10 (1995), de Vries and de Waal Malefyt, eds., Landes Co, Austin Tex.). IL-10 also inhibits IL-12 production by these cells. IL-12 promotes T lymphocyte activation and the differentiation of Th1 lymphocytes (D'Andrea, et al. (1993), J. Exp. Med., 178: 1041-1048; Hsieh et al. (1993), Science, 260: 547-549). In addition, IL-10 directly inhibits T lymphocyte proliferation by inhibiting IL-2 gene transcription and IL-2 production by these cells (reviewed in Interleukin 10 (1995), de Vries and de Waal Malefyt, eds., Landes Co, Austin Tex.), and itself promotes antigen-presenting cells that induce regulatory T cells (U.S. Pat. No. 6,277,635). Thus, in some embodiments, the presence of anergic T lymphocytes may be determined by assaying IL-10 production, e.g. by ELISA in cell supernatants, or by flow cytometric analysis of intracellular staining.

In some embodiments, the immune-modulating agent is an antigen-binding molecule that is immuno-interactive with the target antigen. In some embodiments, the target antigen is expressed in a disease or condition or by a specific pathogen for which an enhanced immune response is required. In other embodiments, the target antigen is aberrantly expressed, typically at a higher level in the disease or condition as compared to the normal state or to a state in which the disease or condition is absent. The antigen-binding molecule is suitably interactive with a target antigen as previously described. Numerous antigen-binding molecules useful in the present invention are known in the art. In an illustrative example in which colon cancer is the subject of the treatment, the antigen-binding molecule is immuno-interactive with an antigen selected from the Cripto-1protein, Pim-1 protein or an antigen present in a colon cancer cell lysate, as disclosed, for example, in United States Patent Application Publication No. 20040176576.

In some embodiments, the antigen-binding molecule is an antibody, especially a whole polyclonal antibody. Such antibodies may be prepared, for example, by injecting an antigen that corresponds to at least a portion of the target antigen into a production species, which may include mice or rabbits, to obtain polyclonal antisera. Methods of producing polyclonal antibodies are well known to those skilled in the art. Exemplary protocols which may be used are described for example in Coligan et al. (1991), Current Protocols in Immunology, (John Wiley & Sons, Inc), and Ausubel et al. (1998, supra), in particular Section III of Chapter 11.

In lieu of polyclonal antisera obtained in a production species, monoclonal antibodies may be produced using the standard method as described, for example, by Köhler and Milstein (1975, Nature, 256: 495-497), or by more recent modifications thereof as described, for example, in Coligan et al. (1991, supra) by immortalising spleen or other antibody producing cells derived from a production species which has been inoculated with one or more antigens as described above.

The invention also contemplates as antigen-binding molecules Fv, Fab, Fab′ and F(ab′)₂ immunoglobulin fragments. Alternatively, the antigen-binding molecule may comprise a synthetic stabilised Fv fragment. Exemplary fragments of this type include single chain Fv fragments (sFv, frequently termed scFv) in which a peptide linker is used to bridge the N-terminus or C-terminus of a V_{H} domain with the C-terminus or N-terminus, respectively, of a V_{L} domain. ScFv lack all constant parts of whole antibodies and are not able to activate complement. ScFvs may be prepared, for example, in accordance with methods outlined in Kreber et al (1997, J. Immunol. Methods, 201(1): 35-55). Alternatively, they may be prepared by methods described in U.S. Pat. No. 5,091,513, European Patent No 239,400 or the articles by Winter and Milstein (1991, Nature, 349: 293) and Plückthun et al (1996, In Antibody engineering: A practical approach., 203-252). In another embodiment, the synthetic stabilised Fv fragment comprises a disulfide stabilised Fv (dsFv) in which cysteine residues are introduced into the V_{H} and V_{L} domains such that in the fully folded Fv molecule the two residues will form a disulfide bond between them. Suitable methods of producing dsFv are described for example in Glockscuther et al. (1990), Biochem., 29: 1362-1367; Reiter et al. (1994), J. Biol. Chem., 269: 18327-18331; Reiter et al. (1994), Biochem. 33: 5451-5459; Reiter et al. (1994), Cancer Res., 54: 2714-2718; Webber et al. (1995), Mo. Immunol., 32: 249-258.

### 5. Methods of LSD1 Inhibition

As described in Section 4 above, the proteinaceous molecules of the invention are useful in methods for enhancing an immune response in a subject to a target antigen by an immune-modulating agent and inhibiting PD-L1 and/or PD-L2 activity, including nuclear translocation of PD-L1 and/or PD-L2. Furthermore, the proteinaceous molecules of the invention are useful in methods for the treatment or prevention of a condition involving LSD1 and/or PKC overexpression, such as a cancer.

In accordance with the present invention, the proteinaceous molecules of the invention are useful for the inhibition of LSD1 nuclear translocation. Thus, the proteinaceous molecules of the invention are useful in methods for altering at least one of formation, proliferation, maintenance, EMT or MET of an LSD1 overexpressing cell. The proteinaceous molecules of the invention are useful for inhibiting the proliferation, survival or viability of an LSD1 overexpressing cell. Thus, the proteinaceous molecules of the invention are useful for the treatment or prevention of a condition involving LSD1 overexpression in a subject, such as a cancer, especially breast cancer.

Accordingly, in another aspect of the invention, there is provided a use of the proteinaceous molecule of the invention for therapy, or in the manufacture of a medicament for therapy. The present invention also encompasses a proteinaceous molecule of the invention for use in therapy, or for use as a medicament.

The proteinaceous molecules of the invention are useful for the inhibition of LSD1, particularly the nuclear translocation of LSD1. Thus, the proteinaceous molecules of the invention are useful in methods of inhibiting an activity of LSD1, such as the nuclear translocation of LSD1 or the phosphorylation of LSD1.

Accordingly, it is proposed that the proteinaceous molecules of the invention will, as a result of their inhibitory action on LSD1, be useful in methods of altering at least one of (i) formation; (ii) proliferation; (iii) maintenance; (iv) EMT; or (v) MET of an LSD1 overexpressing cell. In some embodiments, the proteinaceous molecule of the invention results in a reduction, impairment, abrogation or prevention of the (i) formation; (ii) proliferation; (iii) maintenance; or (iv) EMT of an LSD1 overexpressing cell; and/or in the enhancement of (v) MET of an LSD1 overexpressing cell.

The proteinaceous molecules of the invention may be used for treating or preventing a cancer in a subject, wherein the cancer comprises at least one LSD1 overexpressing cell. The cancer may comprise cancer stem cells and non-cancer stem cell tumor cells. In some embodiments, the cancer is selected from breast, prostate, lung, bladder, pancreatic, colon, melanoma, retinoblastoma, liver or brain cancer; especially breast cancer.

In other embodiments, the proteinaceous molecules of the invention are used for treating, preventing and/or relieving the symptoms of a malignancy, particularly a metastatic cancer. In preferred embodiments, the proteinaceous molecules of the invention are used for treating, preventing and/or relieving the symptoms of a metastatic cancer.

Suitable types of metastatic cancer include, but are not limited to, metastatic breast, prostate, lung, bladder, pancreatic, colon, melanoma, retinoblastoma, liver or brain cancer. In some embodiments, the brain cancer is a glioma. In preferred embodiments, the metastatic cancer is metastatic breast cancer.

The proteinaceous molecules are useful in methods involving LSD1 overexpressing cells. In particular embodiments, the LSD1 overexpressing cell is selected from a breast, prostate, testicular, lung, bladder, pancreatic, colon, melanoma, leukemia, retinoblastoma, liver or brain cell; especially a breast cell. In particular embodiments, the LSD1 overexpressing cell is a breast epithelial cell, especially a breast ductal epithelial cell.

In some embodiments, the LSD1 overexpressing cell is a cancer stem cell or a non-cancer stem cell tumor cell; especially a cancer stem cell tumor cell; most especially a breast cancer stem cell tumor cell. In some embodiments, the cancer stem cell tumor cell expresses CD24 and CD44, particularly CD44^{high}, CD24^{low}.

In some embodiments, the methods further comprise detecting overexpression of an LSD1 gene in a tumor sample obtained from the subject, wherein the tumor sample comprises the cancer stem cell tumor cells and optionally the non-cancer stem cell tumor cells, prior to administering the proteinaceous molecule of the invention to the subject.

The proteinaceous molecules of the invention are suitable for treating an individual who has been diagnosed with a cancer, who is suspected of having a cancer, who is known to be susceptible and who is considered likely to develop a cancer, or who is considered to develop a recurrence of a previously treated cancer. The cancer may be hormone receptor negative. In some embodiments, the cancer is hormone receptor negative and is, thus, resistant to hormone or endocrine therapy. In some embodiments where the cancer is breast cancer, the breast cancer is hormone receptor negative. In some embodiments, the breast cancer is estrogen receptor negative and/or progesterone receptor negative.

There are numerous conditions involving LSD1 overexpression or activity, in which the proteinaceous molecule of the invention may be useful. Accordingly, in another aspect of the invention, there is provided the use of a proteinaceous molecule of the invention for treating or preventing a condition in a subject in respect of which LSD1 inhibition is associated with effective treatment. The invention also contemplates a method of treating or preventing a condition in a subject in respect of which LSD1 inhibition is associated with effective treatment. In a further aspect of the invention, there is provided the use of a proteinaceous molecule of the invention in the manufacture of a medicament for treating or preventing a condition in a subject in respect of which LSD1 inhibition is associated with effective treatment. The invention also provides a proteinaceous molecule of the invention for use in treating or preventing a condition in respect of which LSD1 inhibition is associated with effective treatment.

Non-limiting examples of conditions involving LSD1 overexpression or activity and, thus, conditions in which LSD1 inhibition is associated with effective treatment include cancer; sickle cell disease; viral infection such as HIV, herpes simplex virus (e.g. HSV-1 or HSV-2), adenovirus, human papillomavirus, parvovirus, smallpox virus, vaccinia virus, hepadnaviridae, polyoma virus, Epstein-Barr virus, hepatitis virus (e.g. hepatitis B virus), or varicella-zoster virus infection; inflammatory conditions such as atherosclerosis, a respiratory inflammatory disorder (e.g. respiratory distress syndrome, asthma, chronic obstructive pulmonary disease, bronchial hyperresponsiveness, bronchoconstriction, airway inflammation, airway remodelling or cystic fibrosis), chronic inflammatory bowel disease, ulcerative colitis, Crohn's disease, a chronic skin inflammatory disease (e.g. psoriasis or atopic dermatitis), mesangial glomerulonephritis, Kawasaki disease, disseminated intravascular inflammation, Caffey disease, twin reversed arterial perfusion syndrome, allergic vasculitis, arthritis, vasculitis, coronary artery disease, carotid artery disease, transplant vasculopathy, rheumatoid arthritis, hepatic cirrhosis, or nephritis; cardiovascular conditions such as thrombosis, Budd-Chiari syndrome, Paget-Schroetter disease, myocardial infarction, coronary heart disease, coronary artery disease, stroke, heart failure or hypertension; or neurological disorders such as schizophrenia, developmental disorders, depression, epilepsy, drug addiction or neurodegenerative diseases (e.g. Parkinson's disease, Huntington's disease, Alzheimer's disease or dementia). In particular embodiments, the condition is cancer.

In particular embodiments, the methods involve the administration of a further active agent as described in Section 3 supra, such as an additional cancer therapy and/or an anti-infective agent.

### 6. Methods of PKC Inhibition

The amino acid sequence corresponding to residues 108 to 118 of LSD1 comprises a potential phosphorylation site on serine 111. Without wishing to be bound by theory, it is proposed that the proteinaceous molecules of the invention, the sequence of which corresponds to the amino acid sequence comprising said phosphorylation site and surrounding residues, may bind to a PKC, especially PKC-0, thereby inhibiting the phosphorylating activity of said PKC and, consequently, inhibiting the phosphorylation of LSD1. The lack of LSD1 phosphorylation, in turn, is proposed to inhibit the nuclear translocation of LSD1.

Thus, the proteinaceous molecules of the invention are useful for inhibiting the phosphorylating activity of a PKC, such as inhibiting PKC phosphorylation of LSD1 and altering at least one of formation, proliferation, maintenance, EMT or MET of a PKC overexpressing cell. Furthermore, the proteinaceous molecules of the invention are useful for the treatment or prevention of a condition involving PKC overexpression in a subject, such as a cancer.

In particular embodiments, the PKC is PKC-6.

In some embodiments, the proteinaceous molecules of the invention selectively inhibit the phosphorylating activity of PKC-θ over at least one other PKC enzyme or isoform, such as PKC-α, PKC-β, PKC-γ, PKC-δ, PKC-ε, PKC-ζ, PKC-η, PKC-λ, PKC-μ or PKC-ν. In some embodiments, the proteinaceous molecules of the invention selectively inhibit the phosphorylating activity of PKC-θ over the other 10 PKC enzymes. In some embodiments, the proteinaceous molecules of the invention exhibit PKC-θ selectivity of greater than about 2-fold, 5-fold, 10-fold, 20-fold, 50-fold or greater than about 100-fold with respect to inhibition of the phosphorylating activity of another PKC (i.e. a PKC other than PKC-0, such as PKC-α, PKC-β, PKC-γ, PKC-δ, PKC-ε, PKC-ζ, PKC-η, PKC-λ, PKC-μ or PKC-ν). In other embodiments, selective molecules display at least 50-fold greater inhibition towards PKC-θ than towards another PKC. In further embodiments, selective molecules display at least 100-fold greater inhibition towards PKC-θ than towards another PKC. In still further embodiments, selective molecules display at least 500-fold greater inhibition towards PKC-θ than towards another PKC. In yet further embodiments, selective molecules display at least 100-fold greater inhibition towards PKC-θ than towards another PKC. In some embodiments, the proteinaceous molecules of the invention are non-selective inhibitors of the phosphorylating activity of PKC-θ.

The proteinaceous molecules of the invention are useful in methods of altering at least one of (i) formation; (ii) proliferation; (iii) maintenance; (iv) EMT; or (v) MET of a PKC overexpressing cell. Preferably the cell is contacted with a formation-, proliferation-, maintenance-, EMT- or MET-modulating amount of a proteinaceous molecule of the invention. In some embodiments, the proteinaceous molecule of the invention results in a reduction, impairment, abrogation or prevention of the (i) formation; (ii) proliferation; (iii) maintenance; or (iv) EMT of a PKC overexpressing cell; and/or in the enhancement of (v) MET of a PKC overexpressing cell.

Accordingly, the proteinaceous molecules of the invention may be used for treating or preventing a cancer in a subject, wherein the cancer comprises at least one PKC overexpressing cell. In preferred embodiments, the PKC is PKC-6.

The cancer may be selected from, but is not limited to, breast, prostate, lung, bladder, pancreatic, colon, melanoma, retinoblastoma, liver or brain cancer; especially breast cancer. In particular embodiments, the cancer is a metastatic cancer, especially a metastatic breast cancer.

In some embodiments, the proteinaceous molecules of the invention are used for treating, preventing and/or relieving the symptoms of a malignancy, particularly a metastatic cancer. In preferred embodiments, the proteinaceous molecules of the invention are used for treating, preventing and/or relieving the symptoms of a metastatic cancer.

In preferred embodiments, the PKC is PKC-θ. Suitable PKC-θ overexpressing cells may include, but are not limited to, breast, prostate, lung, bladder, pancreatic, colon, melanoma, liver, retina or glioma cells; especially breast cells. In particular embodiments, the PKC-θ overexpressing cell is a breast epithelial cell, especially a breast ductal epithelial cell.

In particular embodiments, the PKC-θ overexpressing cell is a cancer stem cell or a non-cancer stem cell tumor cell; preferably a cancer stem cell tumor cell. In some embodiments, the CSC tumor cell expresses CD24 and CD44, particularly CD44^{high}, CD24^{low}.

The proteinaceous molecules of the invention are suitable for treating an individual who has been diagnosed with a cancer, who is suspected of having a cancer, who is known to be susceptible and who is considered likely to develop a cancer, or who is considered to develop a recurrence of a previously treated cancer. The cancer may be hormone receptor negative. In some embodiments, the cancer is hormone receptor negative and is, thus, resistant to hormone or endocrine therapy. In some embodiments where the cancer is breast cancer, the breast cancer is hormone receptor negative. In some embodiments, the breast cancer is estrogen receptor negative and/or progesterone receptor negative.

There are numerous conditions involving PKC overexpression, especially PKC-θ overexpression, in which the proteinaceous molecule of the invention may be useful. Accordingly, in another aspect of the invention, there is provided the use of a proteinaceous molecule of the invention for treating or preventing a condition in a subject in respect of which PKC inhibition is associated with effective treatment. The invention also contemplates a method of treating or preventing a condition in a subject in respect of which PKC inhibition, particularly PKC-θ inhibition, is associated with effective treatment. In a further aspect of the invention, there is provided the use of a proteinaceous molecule of the invention in the manufacture of a medicament for treating or preventing a condition in a subject in respect of which PKC inhibition is associated with effective treatment. The invention also provides a proteinaceous molecule of the invention for use in treating or preventing a condition in respect of which PKC inhibition is associated with effective treatment.

Conditions involving PKC overexpression or activity, particularly PKC-θ overexpression or activity, and, thus, conditions in which PKC inhibition is associated with effective treatment include, but are not limited to, cancer; neurological and vascular disorders such as Down's syndrome, memory and cognitive impairment, dementia, amyloid neuropathies, brain inflammation, stroke, Parkinson's disease, nerve and brain trauma, vascular amyloidosis, depression or cerebral hemorrhage with amyloidosis; acute and chronic airway disorders such as bronchitis, obstructive bronchitis, spastic bronchitis, allergic bronchitis, allergic asthma, bronchial asthma, emphysema or chronic obstructive pulmonary disease (COPD); cardiac disorders such as heart failure, atherosclerosis, cardiac fibrosis, hypertrophy or ischemic heart disease; dermatoses such as psoriasis, toxic and allergic contact eczema, atopic eczema, seborrheic eczema, lichen simplex, sunburn, pruritis in the anogenital area, alopecia areata, hypertrophic scars, discoid lupus erythematosus, follicular and wide-area pyodermias, endogenous and exogenous acne or acne rosacea; arthritic conditions such as rheumatoid arthritis, rheumatoid spondylitis, osteoarthritis or other arthritic conditions; acquired immunodeficiency syndrome (AIDS); HIV infection; septic shock; adult respiratory distress syndrome; graft-versus-host reactions; acute or chronic rejection of organ or tissue allografts or xenografts; Crohn's disease; ulcerative colitis; inflammatory bowel disease; allergic rhinitis or sinitis; allergic conjunctivitis; nasal polyps; autoimmune disorders such as multiple sclerosis; kidney disease; or diabetes insipidus. In particular embodiments, the condition is cancer.

In some embodiments, the methods further comprise detecting overexpression of a PKC gene, especially a PKC-6 gene, in a tumor sample obtained from the subject, wherein the tumor sample comprises the cancer stem cell tumor cells and optionally the non-cancer stem cell tumor cells, prior to administering the proteinaceous molecule of the invention to the subject.

In particular embodiments, the methods involve the administration of a further active agent as described in Section 3 supra, such as an additional cancer therapy and/or an anti-infective agent.

A skilled person would be well aware of suitable assays used to evaluate LSD1, PDL-1, PDL-2 and/or PKC inhibition, such as inhibition of nuclear translocation and phosphorylating activity, and to identify proteinaceous molecules that are LSD1 or PKC inhibitors. Screening for active agents according to the invention can be achieved by any suitable method. For example, the method may include contacting a cell expressing a polynucleotide corresponding to a gene that encodes LSD1, PKC, PD-L1 and/or PD-L2 with an agent suspected of having the inhibitory activity and screening for the inhibition of the level or functional activity of LSD1, PKC, PD-L1 and/or PD-L2, or the lowering of the level of a transcript encoded by the polynucleotide, or the inhibition of the activity or expression of a downstream cellular target of the polypeptide or of the transcript (hereafter referred to as target molecules). Detecting such inhibition can be achieved utilizing techniques including, but not restricted to, ELISA, cell-based ELISA, inhibition ELISA, Western blots, immunoprecipitation, immunofluorescence, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a LSD1, PKC, PD-L1 and/or PD-L2 is regulated or expressed may be naturally occurring in the cell which is the subject of testing or it may have been introduced into the host cell for the purpose of testing. In addition, the naturally-occurring or introduced polynucleotide may be constitutively expressed, thereby providing a model useful in screening for agents which down-regulate expression of an encoded product of the sequence wherein the down regulation can be at the nucleic acid or expression product level. Further, to the extent that a polynucleotide is introduced into a cell, that polynucleotide may comprise the entire coding sequence that codes for LSD1, PKC, PD-L1 and/or PD-L2 or it may comprise a portion of that coding sequence (e.g. the active site of LSD1, PKC, PD-L1 and/or PD-L2) or a portion that regulates expression of the corresponding gene that encodes LSD1, PKC, PD-L1 and/or PD-L2 (e.g. a promoter). For example, the promoter that is naturally associated with the polynucleotide may be introduced into the cell that is the subject of testing. In this instance, where only the promoter is utilized, detecting modulation of the promoter activity can be achieved, for example, by operably linking the promoter to a suitable reporter polynucleotide including, but not restricted to, green fluorescent protein (GFP), luciferase, 3-galactosidase and catecholamine acetyl transferase (CAT). Modulation of expression may be determined by measuring the activity associated with the reporter polynucleotide.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which inhibit the expression of an upstream molecule, which subsequently inhibits the expression of the polynucleotide encoding the target molecule. Accordingly, these methods provide a mechanism of detecting agents that either directly or indirectly inhibit the expression or activity of a target molecule according to the invention.

In alternative embodiments, test agents are screened using commercially available assays, illustrative examples of which include EpiQuik Histone Demethylase LSD1 Inhibitor Screening Assay Kit (Epigentek Group, Brooklyn, USA), the LSD1 Inhibitor Screening Assay Kit (Cayman Chemical Company, Ann Arbor, USA), the PKC Kinase Activity Assay Kit (Abcam, Cambridge, United Kingdom), Protein Kinase C Assay Kit (Panvera Corporation, Madison, USA), and Cell-based Immune-checkpoint Assays (Genscript, Piscataway, USA).

Compounds may be further tested in the animal models to identify those compounds having the most potent in vivo effects. These molecules may serve as “lead compounds” for the further development of pharmaceuticals by, for example, subjecting the compounds to sequential modifications, molecular modeling, and other routine procedures employed in rational drug design.

Further suitable assays include the assays described in Sutcliffe et al. (2012) Front Immunol, 3: 260; Ghildyal et al. (2009) J Virol, 83(11): 5353-5362; Riss et al. (2013) Cell Viability Assays, In: Sittampalam, et al., Assay Guidance Manual [Internet]. Bethesda (Md.): Eli Lilly & Company and the National Center for Advancing Translational Sciences, available from: http://www.ncbi.nlm.nih.gov/books/NBK144065/; US 2005222186; Li et al. (2011) J Biomol Screen, 16(2): 141-154; Zhang et al. (2010) FEBS Letters, 584(22): 4646-4654; Johnson and Hunter (2005) Nat Methods, 2(1): 17-25; Peck (2006) Plant J, 45: 512-522; Phillips et al. (2015) Appl Immunohistochem Mol Morphol., 23(8): 541-549; and Satelli et al. (2016) Sci Rep, 6: 28910.

In order that the invention may be readily understood and put into practical effect, particular preferred embodiments will now be described by way of the following non-limiting examples.

### EXAMPLES

### Example 1—Synthesis of Peptide Inhibitors

The LSD1 peptide inhibitors, L1, L2 and L3 (refer to Table 4) were synthesized using automated modern solid phase peptide synthesis and purification technology using the mild Fmoc chemistry method, for example, as described in Ensenat-Waser, et al. (2002) IUBMB Life, 54:33-36 and WO 2002/010193. Couplings were performed using standard N,N′-diisopropylcarbodiimide (DIC)/hydroxybenzotriazole (HOBt) coupling. Following deprotection, peptides were purified using automated preparative reversed phase-high performance liquid chromatography (RP-HPLC). Fractions were analyzed using analytical RP-HPLC and mass spectrometry. Fractions of 98% purity or higher were combined to give the final product.

All peptides tested were myristoylated through the N-terminal amino group of the N-terminal amino acid. Myristoylation was carried out by covalently coupling myristic acid to the N-terminal residue using standard DIC/HOBt coupling as described above, prior to deprotection and purification of the peptides.

TABLE 4
Peptide Inhibitors

| | | SEQ |
|---|---|---|
| Peptide | | ID |
| Name | Sequence | NO: |
| L1 | Myristoyl-RRTSRRKRAKV-OH | 59 |
| L2 | Myristoyl-RRTARRKRAKV-OH | 60 |
| L3 | Myristoyl-RWRRTARRKRAKV-OH | 61 |

### Example 2—Interplay of Phosphorylated LSD1 and PD-L1 and PD-L2 in Breast Cancer Cell Lines

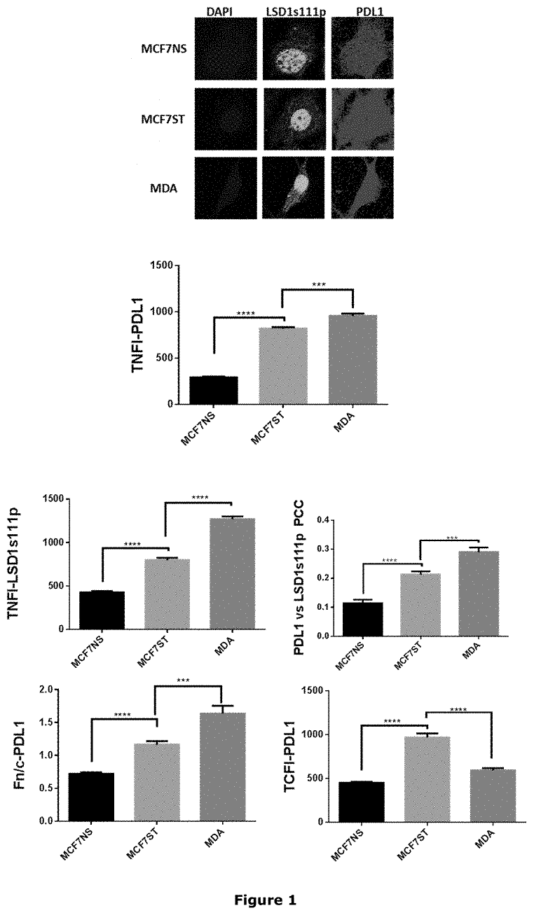

Despite PD-L1 traditionally being described as a cell-surface signaling protein, microscopic analysis demonstrated that PD-L1 has a clear nuclear signal in MCF7 and MDA-MB-231 cells (FIG. 1). PD-L1 has a significantly more nuclear distribution in the mesenchymal MCF7 and the more aggressive triple negative MDA-MB-231 breast cancer cell lines. Both the Fn/c (the ratio of nuclear to cytoplasmic fluorescence) and TNFI (total nuclear fluorescence) clearly show the more nuclear presence, along with a clear cytoplasmic presence (TCFI; total cytoplasmic fluorescence). In addition, there is a clear and almost total nuclear signal for LSD1 phosphorylated at serine 111 (LSD1s111p), which is significantly higher in both the mesenchymal stimulated MCF7 cells (MCF7ST) and the more aggressive cell line MDA-MB-231 (MDA). Additionally, the PCC (co-localization coefficient which measures the degree of co-localization of two proteins within the nucleus of a cell) of LSD1s111p and PD-L1 was low in the epithelial non-stimulated MCF7 cells (MCF7NS), but was significantly increased in the mesenchymal MCF7ST. The highest PCC was in the MDA-MB-231 cells. This data indicates a direct relationship between LSD1s111p and PD-L1 in the nucleus of a cancer cell.

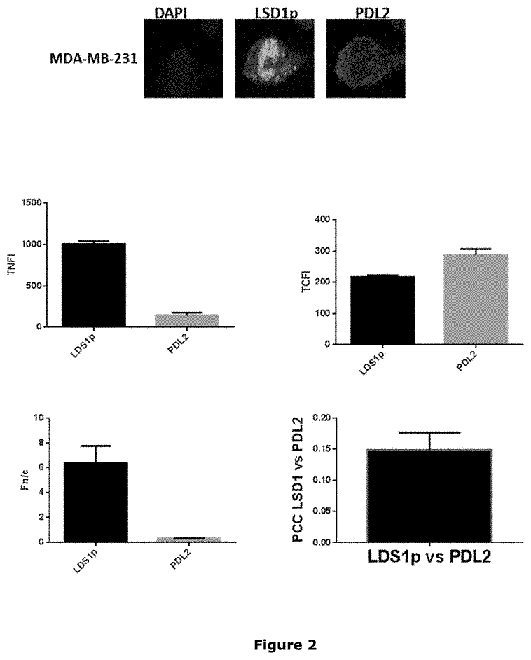

PD-L2 showed a minor nuclear signal in MDA-MB-231 cells, but a positive PCC with LSD1s111p (FIG. 2). This data indicates a nuclear presence of PD-L2 and a direct relationship between LSD1s111p and PD-L2 in the nucleus of a cancer cell.

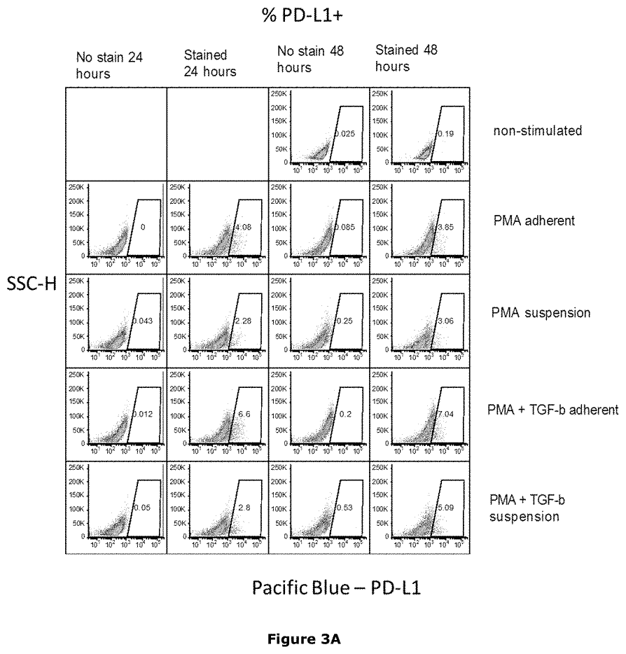

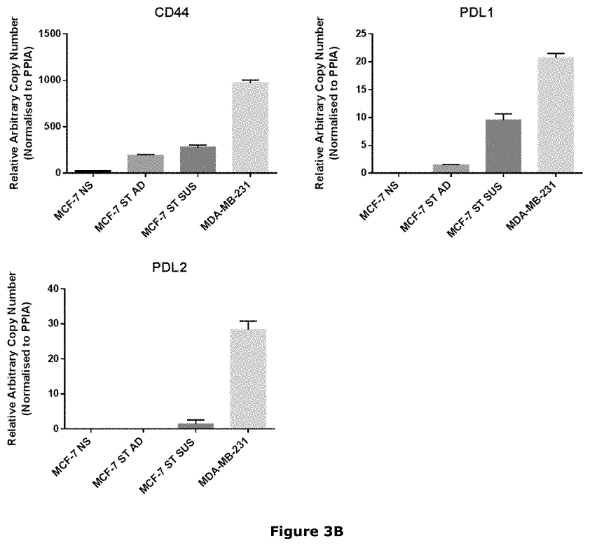

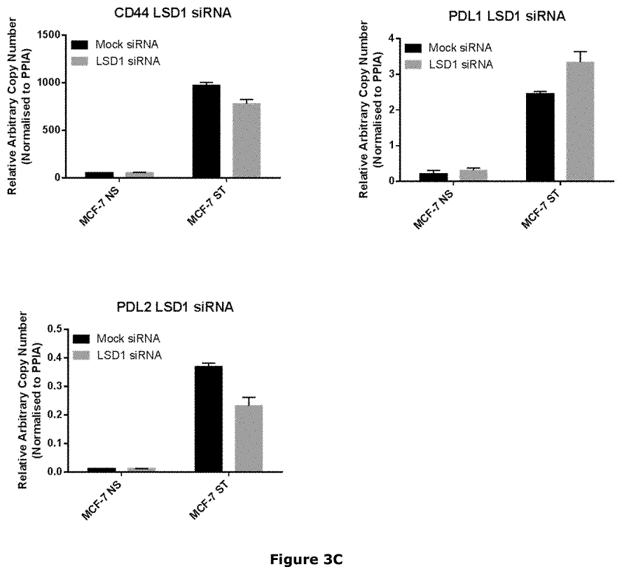

FIG. 3A depicts the FACS plots of inducible MCF7s stimulated with PMA or PMA and TGF-3 that have been stained with PD-L1. The mRNA of these cells was examined for PD-L1, PD-L2 and CD44. PD-L1 mRNA expressed higher in MCF7 cells compared to PD-L2 (FIG. 3B). Conversely, PD-L2 is expressed slightly higher in MDA-MB-231 cells compared to PD-L1. Both PD-L1 and PD-L2 induced upon stimulation of MCF7 cells. PD-L1 and PD-L2 expression is significantly higher in MDA-MB-231 cells compared with MCF7 cells. As with CD44, PD-L1 and PD-L2 mRNA is expressed at a greater level in the floating cells (SUS) compared with the adherent population (AD), which is opposite to the FACS results. When treated with LSD1 inhibitors (LSD1 siRNA), it was found that PD-L2 and CD44 mRNA expression is down-regulated by LSD1 siRNA, whereas PD-L1 expression increases (FIG. 3C).

Table 5 depicts the stimulation of MDA-MB-231 breast cancer cells with Jurkat-T-cells. The frequency (%) of PD-L1+ cells is not altered by stimulation with IFNγ (10 μg/mL), PMA (24 ng/mL) or PMA+IFNγ. Similarly, the frequency of PD-L1+ cells remains unchanged regardless of whether cells are stimulated for 24 or 48 hours.

Stimulation of MDA-MB-231 cells for 24 hours with IFNγ alone resulted in a population shift and decrease in frequency of CD44^{hi}/CD24^{lo} cells to 77.7%. None of the stimulus combinations significantly changed the frequency of CD44^{hi}/CD24^{lo} PD-L1+ MDA-MB-231 cells. The Median Fluorescence Intensity (MFI) of the cells increases with IFNγ stimulation and is decreased by PMA stimulation alone (Table 6). Stimulation with both PMA and IFNγ further increased the MFI of PD-L1.

TABLE 5
Stimulation of MDA-MB-231 breast cancer cells
with Jurkat-T-cells (Frequency of parent, %).

| | Subset population (Frequency of parent, %) | | |
|---|---|---|---|
| | | | CD44^{hi} CD24^{lo}, |
| Sample | PD-L1+ | CD44^{hi} CD24^{lo} | PD-L1+ |
| Non-stimulated | 0.42 | 0.23 | 0 |
| MCF7 | | | |
| Non-stimulated | 99.0 | 99.4 | 99.0 |
| MDA-MB-231 | | | |
| IFNγ stimulated | 99.7 | 77.7 | 99.7 |
| MDA-MB-231 24 | | | |
| hours | | | |
| IFNγ stimulated | 99.7 | 98.9 | 99.7 |
| MDA-MB-231 48 | | | |
| hours | | | |
| PMA stimulated | 98.5 | 98.4 | 98.6 |
| MDA-MB-231 24 | | | |
| hours | | | |
| PMA stimulated | 99.1 | 98.3 | 99.2 |
| MDA-MB-231 48 | | | |
| hours | | | |
| IFNγ and PMA | 99.7 | 99.4 | 99.7 |
| stimulated MDA-MB- | | | |
| 231 24 hours | | | |
| IFNγ and PMA | 98.7 | 98.5 | 99.4 |
| stimulated MDA-MB- | | | |
| 231 48 hours | | | |

TABLE 6
Stimulation of MDA-MB-231 breast cancer cells with Jurkat-T-cells
(Mean Fluorescence Intensity of Comp Pacific Blue-A PD-L1).

Subset population (Mean
Fluorescence Intensity)

| | | CD44^{hi} CD24^{lo}, |
|---|---|---|
| Sample | PD-L1+ | PD-L1+ |
| Non-stimulated MCF7 | 1373 | N/A |
| Non-stimulated MDA-MB- | 4125 | 4133 |
| 231 | | |
| IFNγ stimulated MDA-MB- | 6015 | 6173 |
| 231 24 hours | | |
| IFNγ stimulated MDA-MB- | 6133 | 6173 |
| 231 48 hours | | |
| PMA stimulated MDA-MB- | 3126 | 3145 |
| 231 24 hours | | |
| PMA stimulated MDA-MB- | 3983 | 4016 |
| 231 48 hours | | |
| IFNγ and PMA stimulated | 7033 | 7049 |
| MDA-MB-231 24 hours | | |
| IFNγ and PMA stimulated | 9929 | 9974 |
| MDA-MB-231 48 hours | | |

This data indicates that stimulation of Jurkat cells in the presence of MDA-MB-231 cells will not alter the frequency of PD-L1 or CD44^{hi}/CD24^{lo} populations, but may alter the amount of PD-L1 expressed on the cell surface.

### Example 3—Effect of LSD1 Peptide Inhibitors on the Expression and Nuclear Dynamics of LSD1 and PD-L1

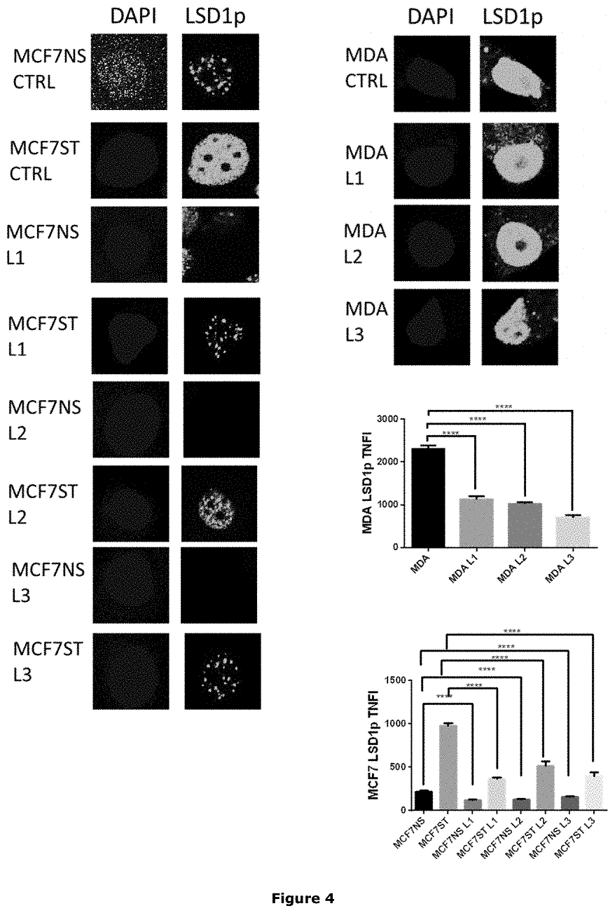

The effect of the peptide inhibitors L1, L2 and L3 (synthesized according to the method of Example 1) on the expression and nuclear localization of LSD1s111p in MCF7 and MDA-MB-231 cells was assessed using confocal laser scanning microscopy (FIG. 4). All peptide inhibitors significantly abrogated nuclear expression of LSD1s111p relative to the control stimulated and non-stimulated MCF7 and MDA-MB-231 cells. Thus, it is evident that the LSD1 peptide inhibitors inhibit LSD1s111p expression and nuclear localization.

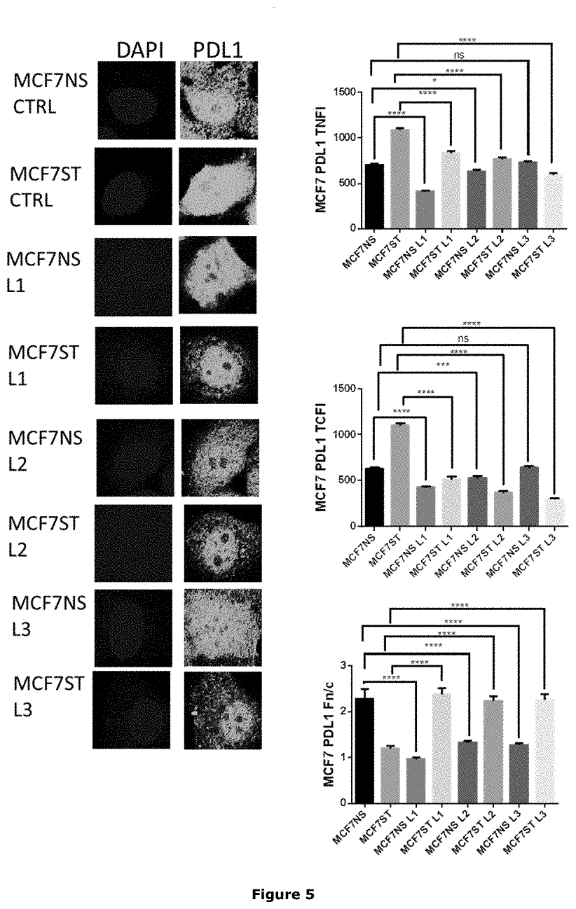

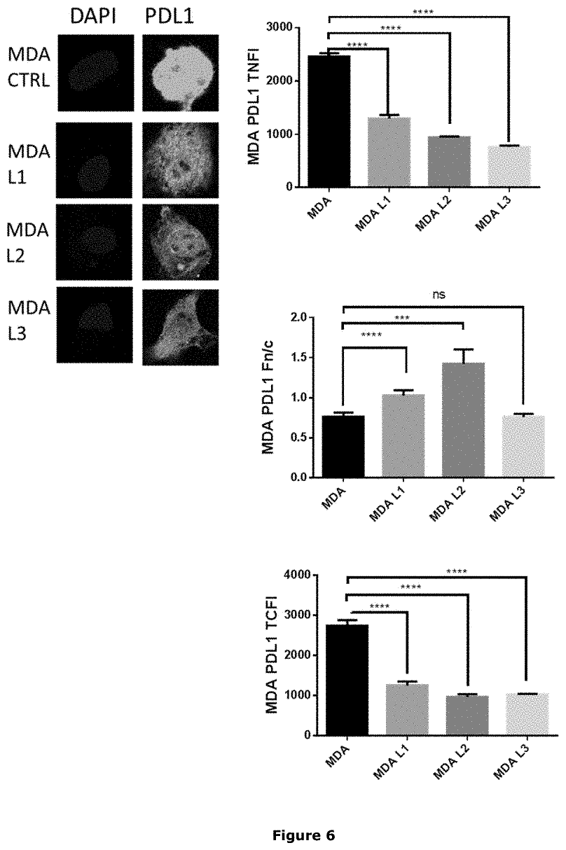

Following this, the effect of the peptide inhibitors L1, L2 and L3 on the expression and nuclear localization of PD-L1 in MCF7 (FIG. 5) and MDA-MB-231 cells (FIG. 6) was determined confocal laser scanning microscopy. All three peptides significantly inhibited the cytoplasmic/surface expression of PD-L1 in both stimulated MCF7 cells and MDA-MB-231 cells. Upon stimulation of the control MCF7 cells there is significant expression of nuclear PD-L1 as well as high levels of nuclear expression of PD-L1. This is also evident in MDA-MB-231 cells. This, PD-L1 clearly has a strong nuclear presence in aggressive breast cancer cell lines. When treated with L1, L2 and L3, the expression of nuclear PD-L1 was significantly abrogated in both stimulated, mesenchymal MCF7 cells and MDA-MB-231 cells. This effect is lessened in the epithelial non-stimulated MCF7 cells. The Fn/c ratio which measures the nuclear bias clearly shows that, even though there is significant abrogation in the L1, L2 and L3 treated samples of both cytoplasmic and nuclear PD-L1, the bias for the localization of PD-L1 is clearly strongly nuclear for the MCF7 and MDA-MB-231 cells.

### Example 4—In Vivo Mouse MDA-MB-231 Xenografts and the Effect of Combination Therapy on the LSD1/PD-L1 Regulation Axis

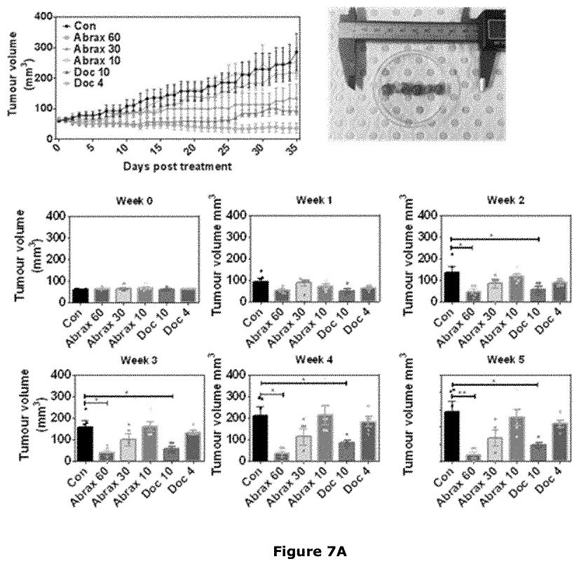

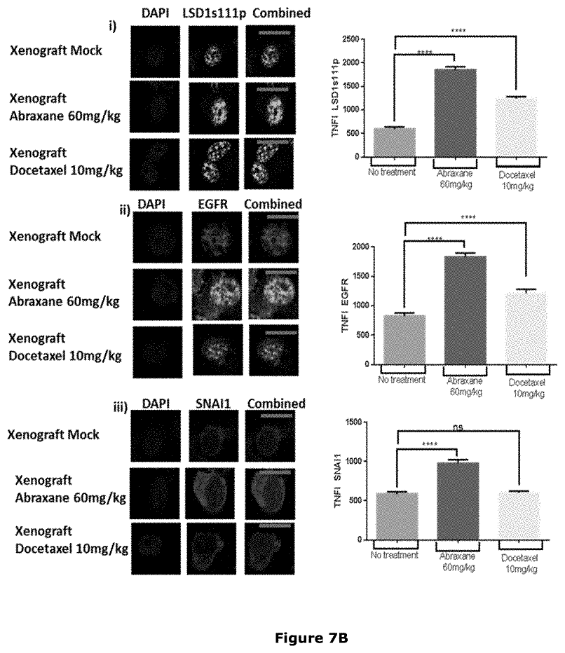

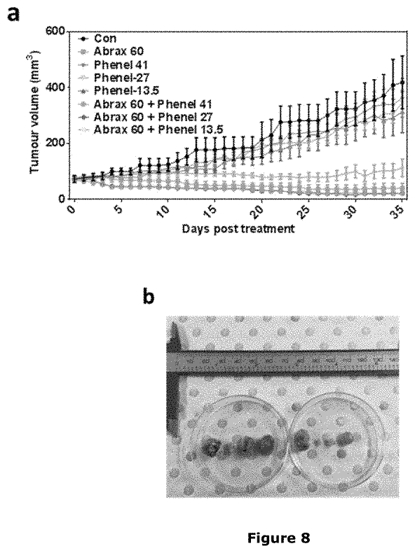

FIG. 7 depicts the effect of treatment of a mouse MDA-MB-231 xenograft with either Abraxane (60 mg/kg) or Docetaxel (10 mg/kg) showing the volume of the tumors over time during treatment (FIG. 7A). Surviving chemo-resistant MDA-MB-231 cells in either the Abraxane or Docetaxel treated cells exhibited a significantly increased fluorescent signal of LSD1s111p [FIG. 7B (i)], EGFR [FIG. 7B (ii)] or SNAIL [FIG. 7B (iii)] relative to xenograft MDA-MB-231 cells treated with vehicle alone when analysed using confocal laser scanning microscopy.

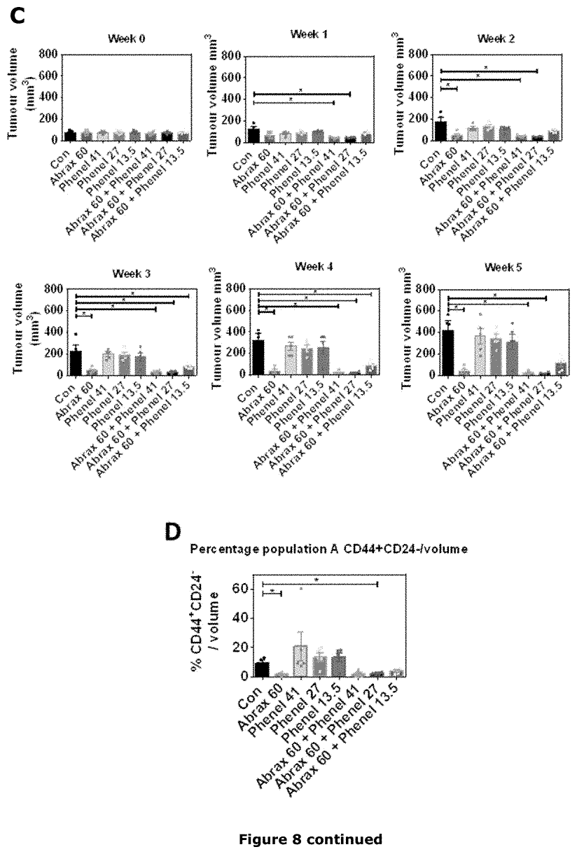

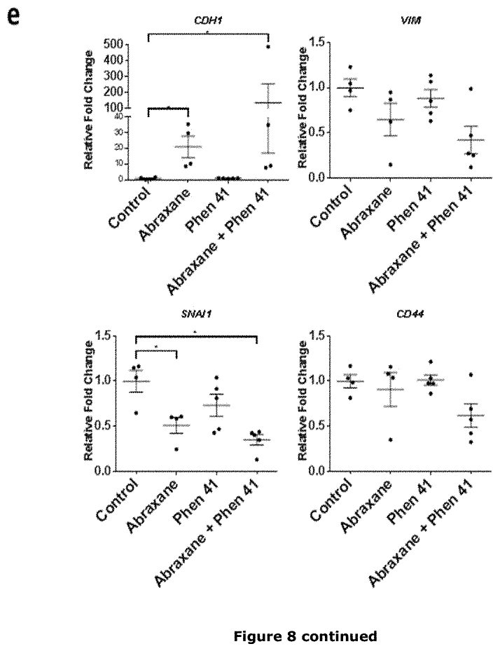

FIG. 8 depicts the effect of treatment with Abraxane, Phenelzine or a combination thereof on xenograft MDA-MB-231 cells. The effect of the combination treatment on the size and volume of the tumors for each of the group over time was assessed. Treatment with either Abraxane alone or in combination with Phenelzine resulted in a significant reduction of tumor volume. Along with CD44, SNAIL and vimentin showing a significant drop as indicated.

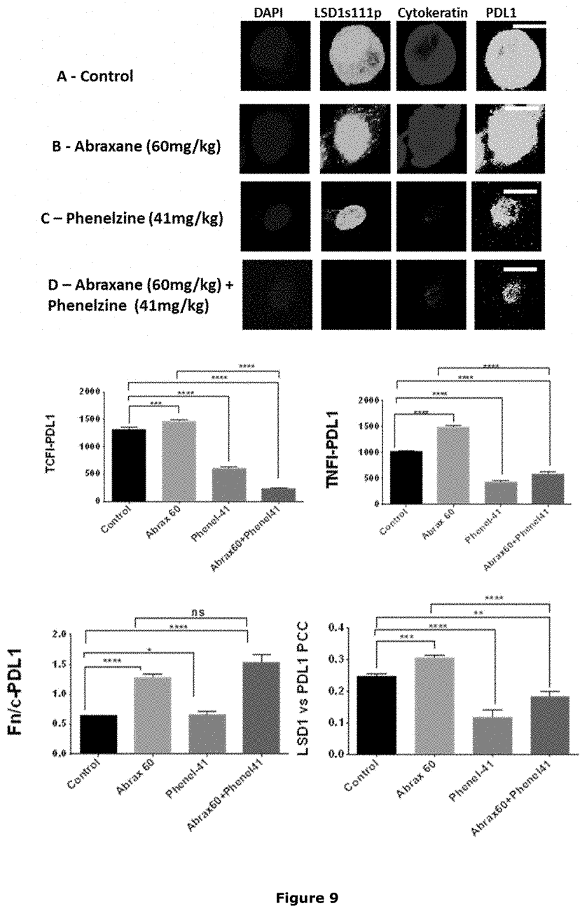

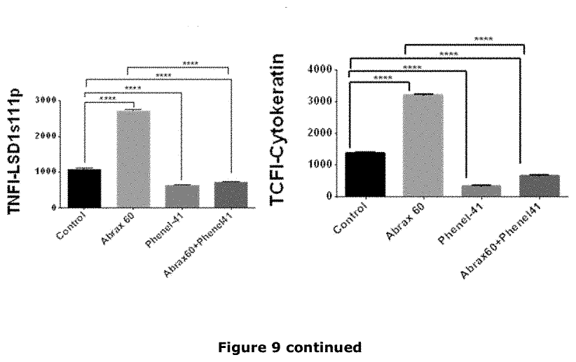

Microscopy analysis of LSD1s111p expression in the MDA-MB-231 cells treated with Abraxane, Phenelzine or a combination thereof found that LSD1s111p expression was significantly increased relative to the control (Group A) in the Abraxane treated samples (Group B—Abraxane 60 mg/kg), suggesting a resistant population of MDA xenograft cells (FIG. 9). Conversely, treatment with Phenelzine (41 mg/kg; Group C) caused an inhibitory effect relative to the control. In the cells treated with both Abraxane and Phenelzine (Group D; Abraxane 60 mg/kg, Phenelzine 41 mg/kg), the expression of LSD1s111p was significantly abrogated compared to Group A (control) and Group B. An almost identical expression profile is noted for cytokeratin, wherein its expression is increased by the Group B treatment and abrogated in cells treated with Phenelzine, and Phenelzine and Abraxane. PD-L1 was also significantly upregulated in both the nucleus (TNFI) and cytoplasm (TCFI) in Abraxane treated cells and a higher nuclear bias was observed (as measured by Fn/c). Treatment with Phenelzine or both Abraxane and Phenelzine significantly abrogated both nuclear (TNFI) and cytoplasmic (TCFI) PD-L1 expression and, although the nuclear bias (Fn/c) was higher, the total PD-L1 expression was significantly reduced.

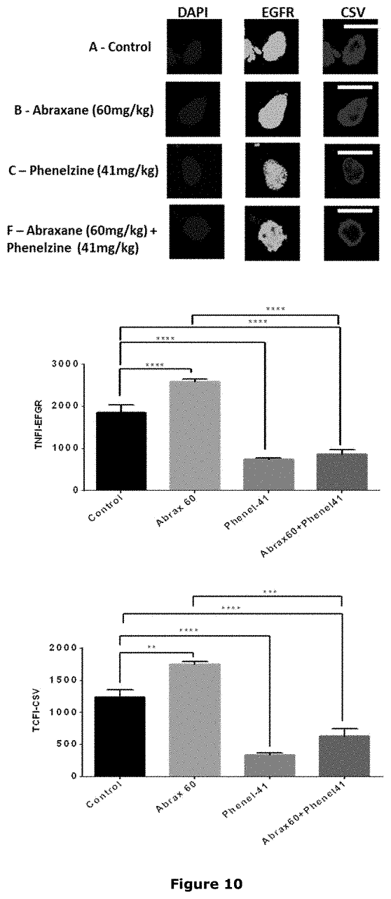

The effect of treatment with Abraxane, Phenelzine or combination of the two on epidermal growth factor receptor (EGFR) and cell surface vimentin (CSV) expression in xenograft MDA-MB-231 cells was assessed using confocal laser scanning microscopy (FIG. 10). EGFR nuclear expression was significantly increased relative to the control (Group A) in the Abraxane treated samples (Group B; Abraxane 60 mg/kg), suggesting a resistant population of MDA-MB-231 xenograft cells. Conversely, treatment with Phenelzine (41 mg/kg; Group C) caused an inhibitory effect relative to the control. In the cells treated with both Abraxane and Phenelzine (Group D; Abraxane 60 mg/kg, Phenelzine 41 mg/kg), the nuclear expression of EGFR was significantly abrogated compared to Group A (control) and Group B. An almost identical expression profile is noted for CSV, wherein its cytoplasmic expression was increased in Group B cells and abrogated in cells treated with Phenelzine and both Phenelzine and Abraxane.

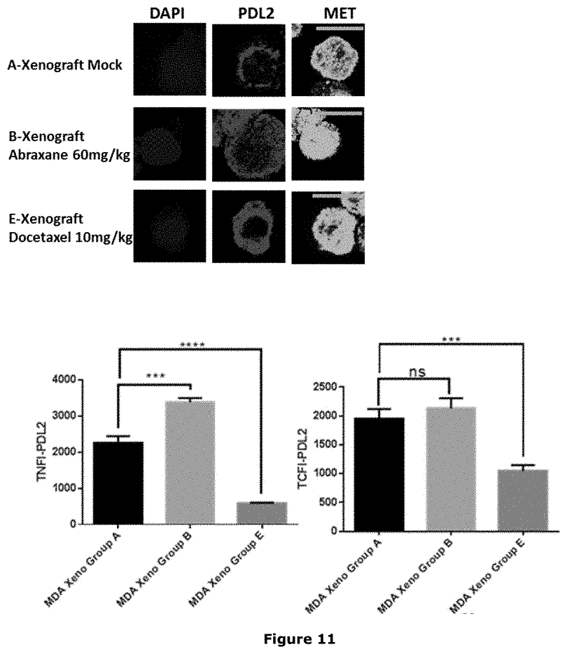

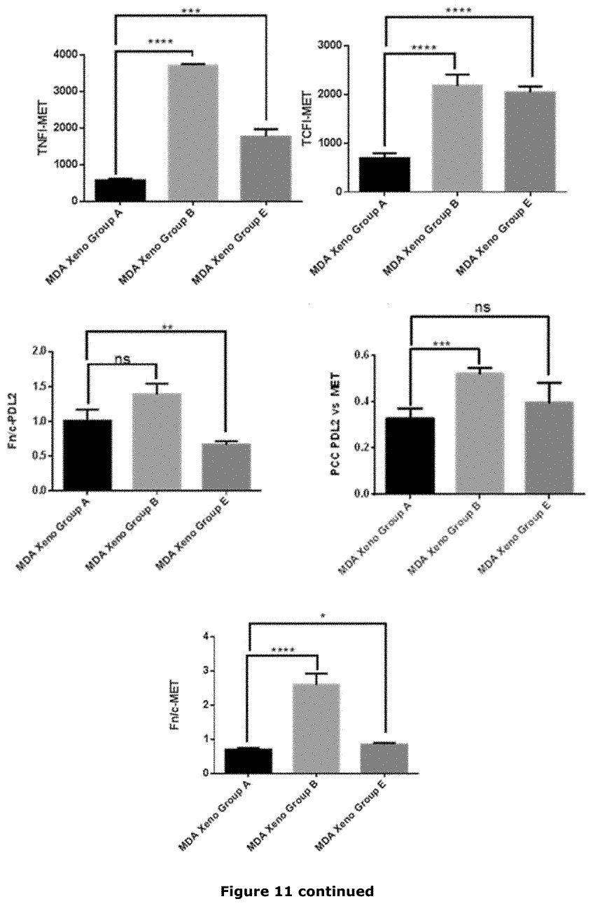

The effect of treatment with Abraxane or Docetaxel on PD-L2 and MET (a mesenchymal marker) expression in xenograft MDA-MB-231 cells was assessed using confocal laser scanning microscopy (FIG. 11). PD-L2 showed a significant nuclear signal and a moderate to strong PCC (localization score) with MET, which was highest in the Abraxane treated cells. MET nuclear expression was also significantly increased in both Abraxane and Docetaxel treated MDA-MB-231 xenograft cells.

### Example 5—the Interplay of LSD1 Phosphorylated at Serine 111 and PD-L1 and PD-L2 in Circulating Tumor Cells Isolated from Metastatic Breast Cancer Patient Liquid Biopsies

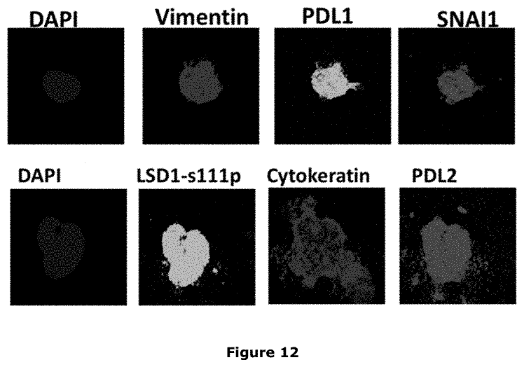

Circulating tumor cells (CTCs) were isolated from meastatic breast cancer patient liquid biopsies. Representative confocal laser scanning microscopy images of isolated CTCs are presented in FIG. 12. The marks detected include SNAIL, a transcription factor implicated in aggressive cancer with a mesenchymal state; vimentin; cytokeratin; LSD1s111p; PD-L1; and PD-L2.

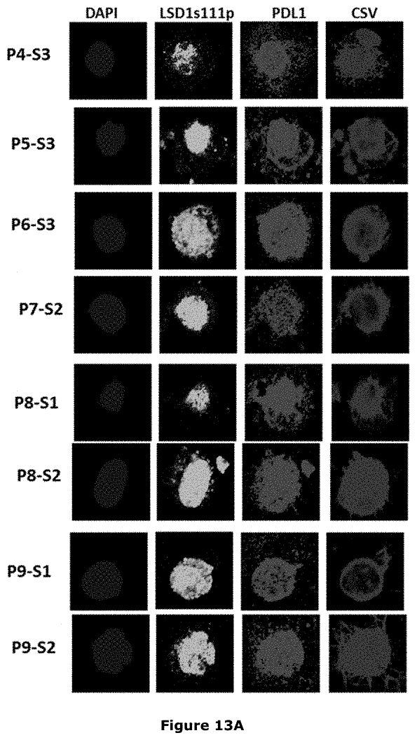

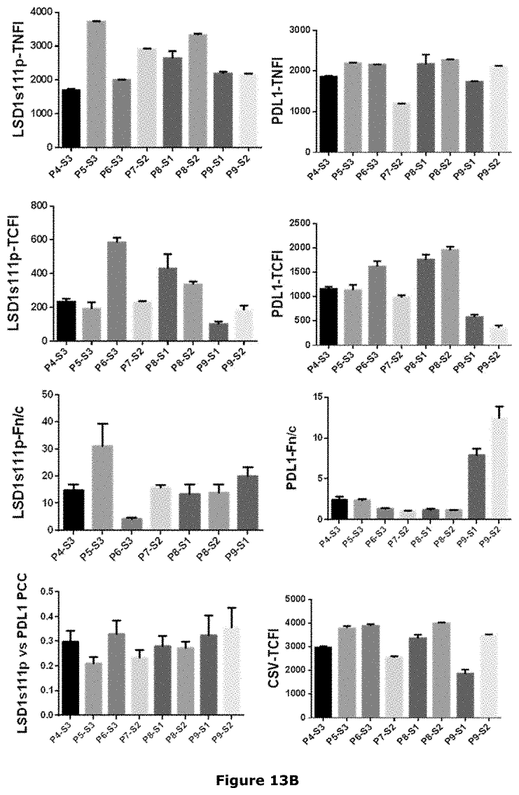

In CTCs isolated from metastatic breast cancer patient liquid biopsies PD-L1 clearly demonstrated a definite and clear nuclear signal when analyzed using confocal laser scanning microscopy (FIG. 13). A clear cytoplasmic/cell surface signal was also detectable. In addition, significant levels of nuclear LSD1s111p was detected in all patient samples. All cells were also positive for cell surface vimentin (CSV), which is a marker for mesenchymal CTCs. Overall, the patient samples all displayed a nuclear PD-L1 signal and the Fn/c showed either a bias towards the nucleus or a parity in signal intensity between the nucleus and cytoplasm. A significant LSD1s111p and PD-L1 positive PCC was observed, which strongly indicates that these two markers interact in the nucleus.

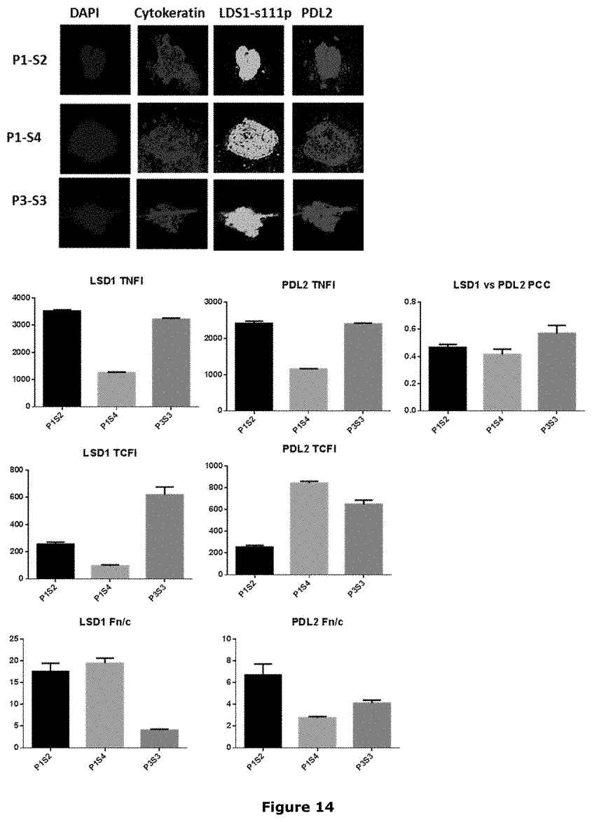

In CTCs isolated from metastatic breast cancer patient liquid biopsies PD-L2, like PD-1, demonstrated a clear nuclear and cytoplasmic/cell surface signal when analyzed using confocal laser scanning microscopy (FIG. 14). Significant levels of nuclear LSD1s111p was detected in all patient samples and all cells were positive for the CTC marker, cytokeratin. A significant LSD1s111p and PD-L2 positive PCC was observed, which strongly indicates that these two markers interact in the nucleus. In summary, the patient samples all displayed a nuclear PD-L2 signal and the Fn/c showed either a bias towards the nucleus or a parity in signal intensity between the nucleus and cytoplasm.

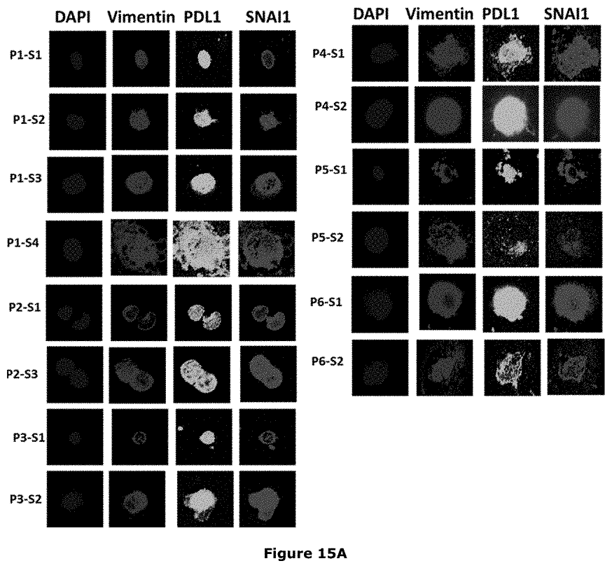

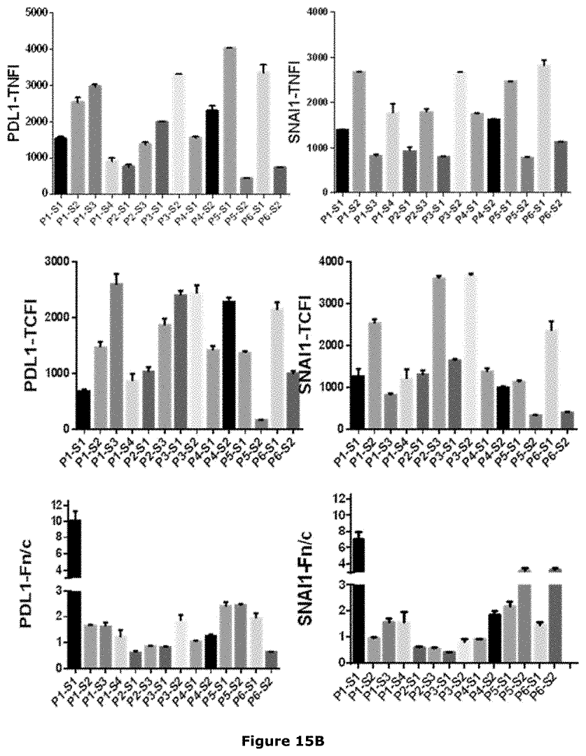

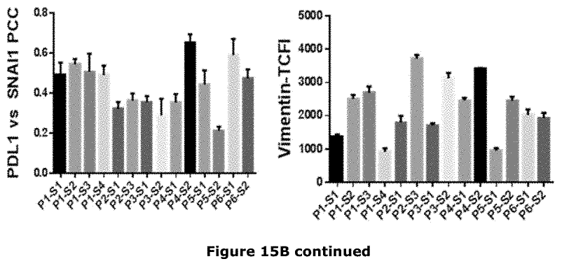

CTCs isolated from metastatic patient breast cancer liquid biopsies were labelled for cell surface vimentin, SNAIL and PD-L1 and analyzed using confocal laser scanning microscopy. PD-L1 clearly demonstrated a clear nuclear and cytoplasmic/cell surface signal (FIG. 15). A general trend of increased nuclear and cytoplasmic signal intensities was observed in patients 1, 2, 3 and 4 whereas patients 5 and 6 displayed a reduction in both nuclear (TNFI) and cytoplasmic (TCFI) fluorescence after the first sample. Overall, the patient samples all displayed a nuclear PD-L1 signal and the Fn/c showed either a bias towards the nucleus or a parity in signal intensity between the nucleus and cytoplasm. Furthermore, a significant SNAIL (a target of LSD1 regulation) and PD-L1 positive PCC was observed, which strongly indicates these two markers interact in the nucleus.

### Example 6—Effect of LSD1 Inhibitors on CTCS Isolated from Metastatic Breast Cancer Patient Liquid Biopsies

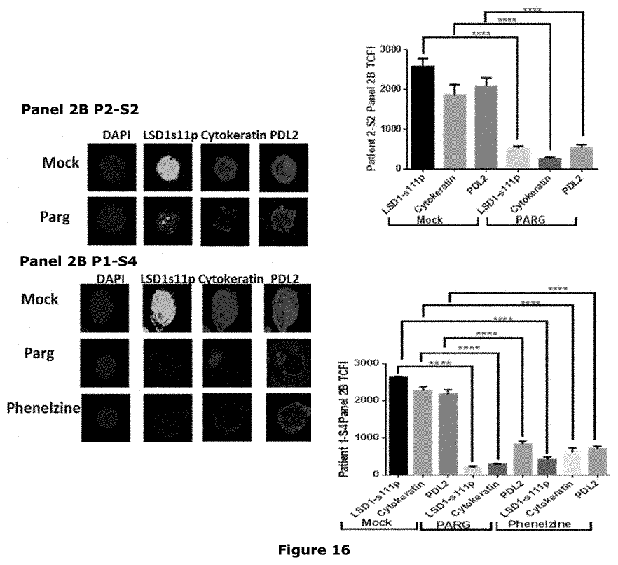

CTCs isolated from two metastatic breast cancer patient liquid biopsies were treated with the LSD1 catalytic inhibitors, Pargyline (Parg) or Phenelzine (FIG. 16). The effects of the LSD1 inhibitors was assessed by analyzing the expression of LSD1s111p, cytokeratin (a marker for CTCs) and PD-L2 using confocal laser scanning microscopy. Both LSD1 catalytic inhibitors, Pargyline and Phenelzine, resulted in a significant knockdown of LSD1s111p, PD-L2 and the CTC marker, cytokeratin. This strongly demonstrates that LSD1 catalytic inhibitors can successfully abrogate LSD1s111p and can knockdown PD-L2 and cytokeratin expression in patient CTCs.

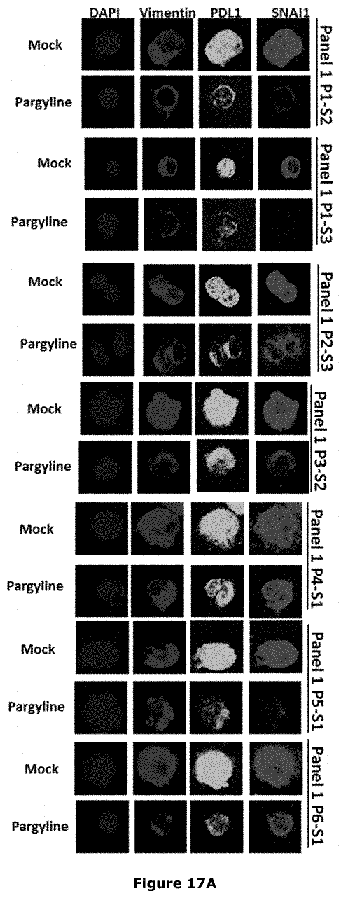

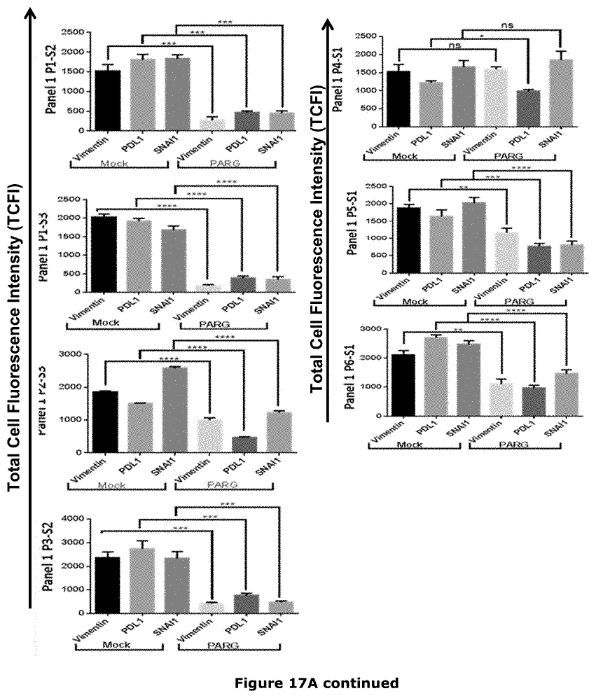

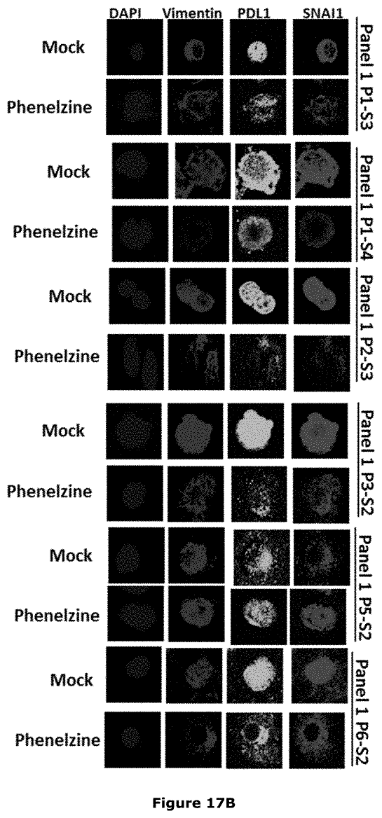

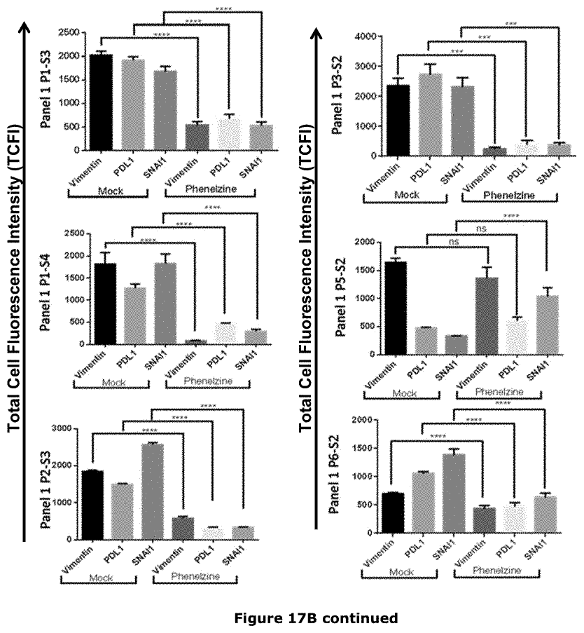

CTCs isolated from metastatic breast cancer patient liquid biopsies were treated with the LSD1 catalytic inhibitor, Pargyline (Parg) (FIG. 17A). The effects of this inhibitor was determined by analyzing the expression of SNAIL (a target of LSD1 regulation), vimentin and PD-L1 using confocal laser scanning microscopy. With the exception of one patient, Pargyline resulted in a significant knockdown of PD-L1 and SNAIL expression. This strongly suggests that LSD1 catalytic inhibitors can abrogate PD-L1 and SNAIL expression in patient CTCs.

CTCs isolated from metastatic breast cancer patient liquid biopsies were treated with the LSD1 catalytic inhibitor, Phenelzine (FIG. 17B). The effect of this inhibitor was determined by analysing the expression of SNAIL and PD-L1 using confocal laser scanning microscopy. Again, as with Pargyline, the LSD1 catalytic inhibitor Phenelzine resulted in a significant knockdown of PD-L1 and SNAIL expression in all but one patient. This data strongly suggests that inhibitors of the catalytic activity of LSD1 can abrogate PD-L1 and SNAIL expression in patient CTCs.

### Example 7—Effect of CDK and LSD1 Inhibitors on CTCS Isolated from Metastatic Breast Cancer Patient Liquid Biopsies

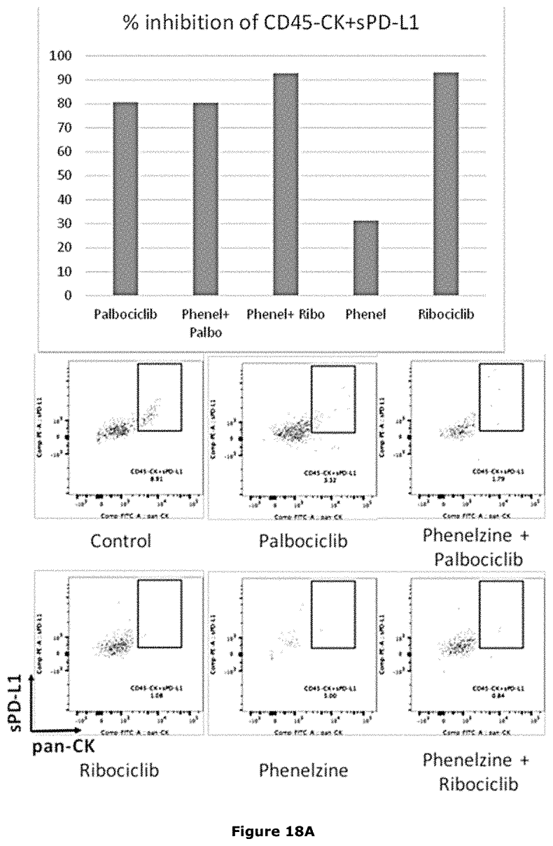

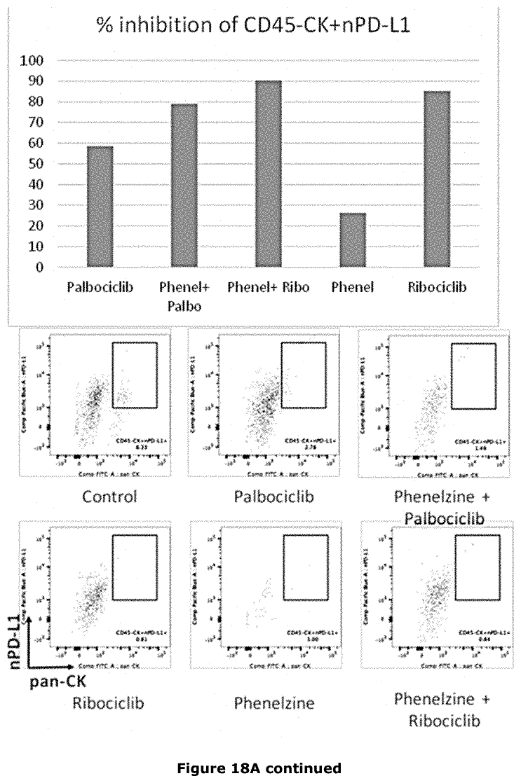

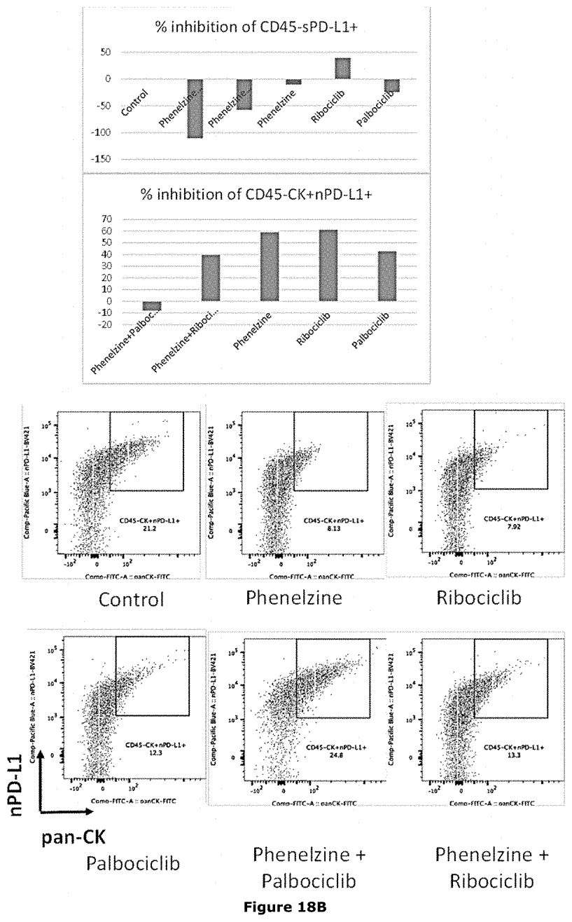

FIGS. 18A and 18B show the effect of the cyclin-dependent kinase (CDK) inhibitors Palbociclib and Ribociclib, and the inhibitor of LSD1 catalytic function, Phenelzine, and combinations thereof on isolated CTCs from metastatic breast cancer patient liquid biopsies using FACS.

In relation to Patient A, 9.26% of CD45− cells were CD45−CK+(Cytokeratin positive) CTCs. However, no CD45−EpCAM+ cells were detected. All test agents and combinations thereof inhibited PD-L1 nuclear (nPD-L1) and surface/cytoplasmic (sPD-L1) expression in CD45−CK+CTCs (FIG. 18A). Ribociclib and the combination of Phenelzine and Ribociclib demonstrated the greatest inhibition of nuclear PD-1.

Turning to Patient B, approximately 14000 CD45−EpCAM+CTCs were detected in PBMCs extracted from equivalent to 2 mL blood. 22.8% of CD45− cells were CD45−CK+CTCs. Again, all test agents and combinations thereof inhibited PD-L1 nuclear expression in CD45−CK+CTCs (FIG. 18B). However, only Ribociclib inhibited both nuclear and surface/cytoplasmic expression of PD-L1.

### Example 8—Effect of LSD1 Peptide Inhibitors on LSD1 Nuclear Translocation in MDA-Mb-231 Cells

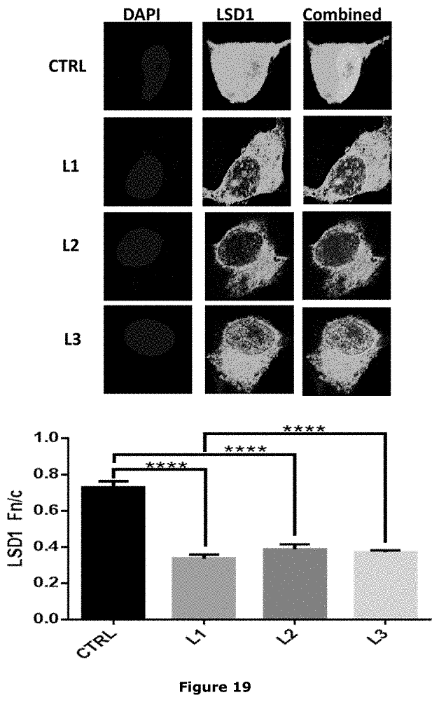

The effect of three peptide LSD1 inhibitors, L1, L2 and L3 (synthesized according to Example 1), on nuclear translocation of LSD1 was assessed in MDA-MB-231 cells using confocal laser scanning microscopy. L1, L2 and L3 inhibited nuclear translocation of LSD1 in MDA-MB-231 cells (FIG. 19).

### Example 9—Effect of LSD1 Peptide Inhibitors on CD44^{HI}CD24^{LO} Cancer Stem Cell Formation

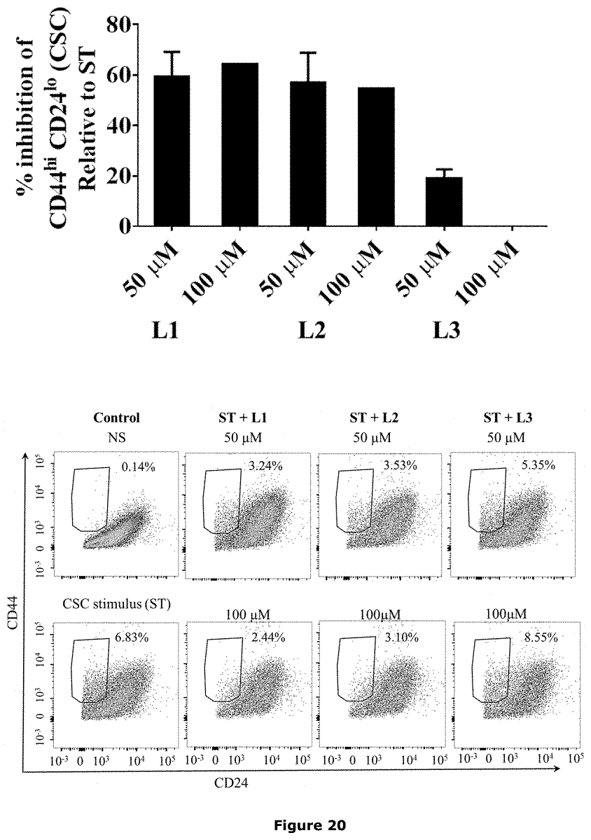

The effect of three peptide LSD1 inhibitors, L1, L2 and L3 (synthesized according to Example 1), on CD44^{hi}CD24^{lo} cancer stem cell formation in PMA stimulated MCF7 cells was assessed using FACS. L1 and L2 inhibited cancer stem cell formation in MCF7 cells (FIG. 20). L3 inhibited approximately 20% of cancer stem cell formation when applied at a concentration of 50 μM.

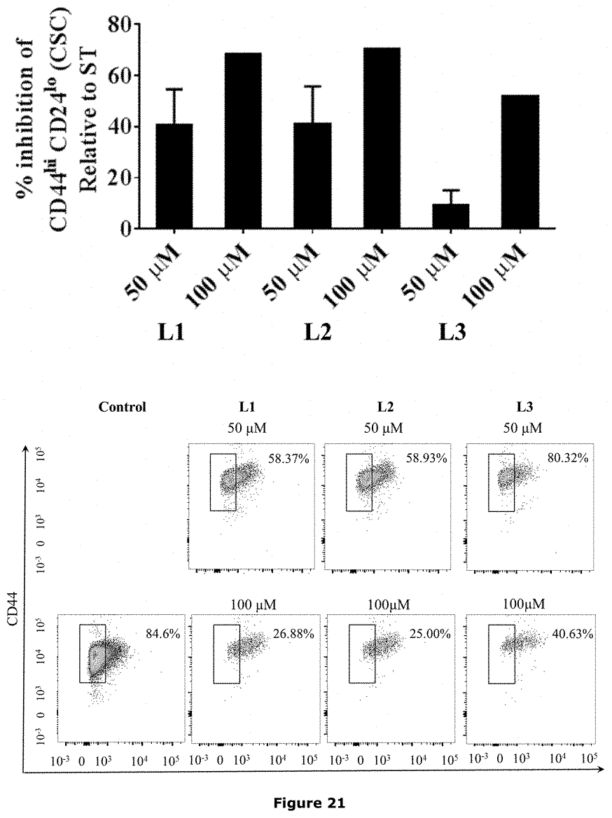

The ability of L1, L2 and L3 to inhibit cancer stem cell formation was also tested in MDA-MB-231 cells. 100 μM L1, L2 and L3 caused a significant reduction of cancer stem cell formation in MDA-MB-231 cells, whereas only L1 and L2 inhibited at least 30% of cancer stem cell formation at a concentration of 50 μM (FIG. 21).

### Example 10—Effect of LSD1 Peptide Inhibitors on PD-L1, EGFR and Met Expression

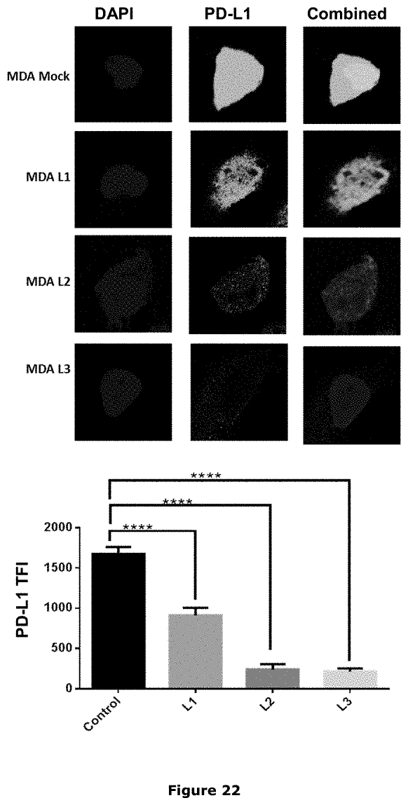

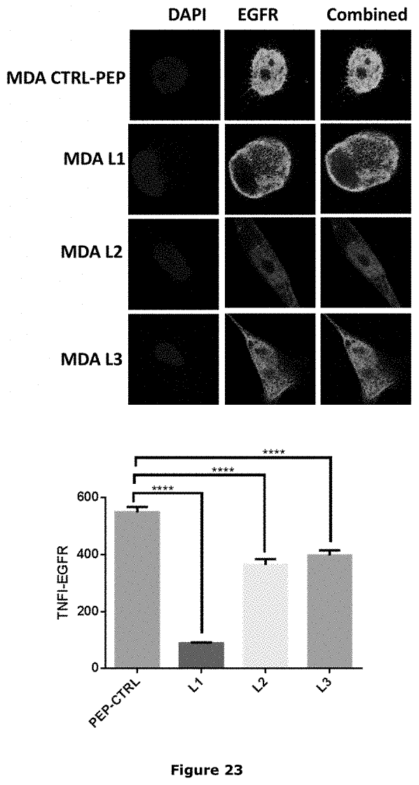

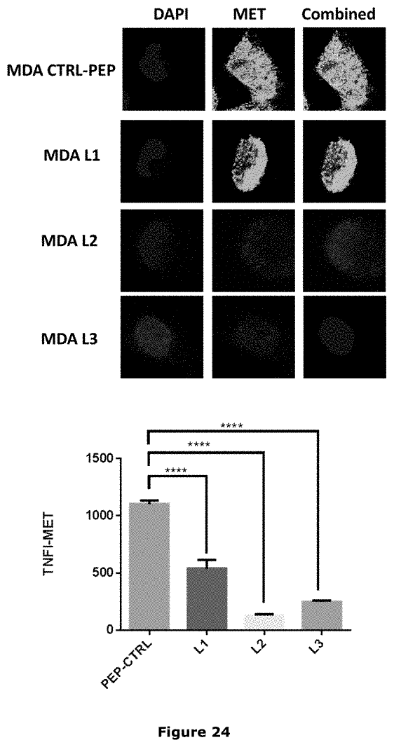

The ability of the peptide inhibitors to modulate PD-L1, EGFR and MET expression was determined in MDA-MB-231 cells using confocal laser scanning microscopy. L1, L2 and L3 (synthesized according to Example 1) abrogated PD-L1 nuclear expression in comparison to the control (FIG. 22). Treatment with L1 resulted in a significant abrogation of EGFR nuclear expression in comparison to the control cells (FIG. 12). L2 and L3 treatment also resulted in a reduction in EGFR nuclear expression, although this was to a lesser extent than L1 (FIG. 23). All three peptides caused a significant abrogation of MET nuclear expression, with L2 and L3 having the most pronounced effect (FIG. 24).

### Example 11—Localization of LSD1 and PKC-Θ within Breast Cancer Cells

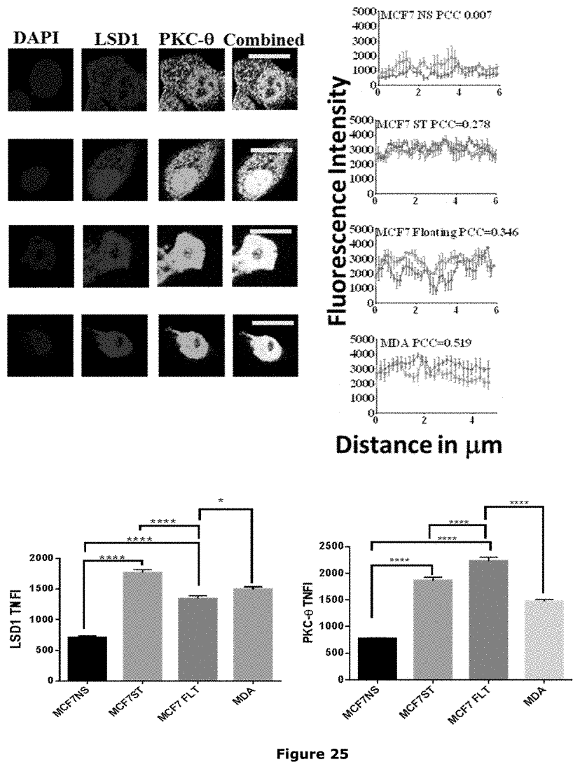

The presence of PKC-θ and LSD1 in MDA-MB-231 cells or MCF7 cells treated with vehicle alone or PMA for 60 hours was determined using confocal laser scanning microscopy (FIG. 25). Both LSD1 and PKC-θ were present in the nucleus of all cells, with their nuclear expression increasing in stimulated MCF7 cells (MCF7ST), floating MCF7 cells (MCF7 FLT), and MDA-MB-231 cells in comparison to non-stimulated MCF7 cells (MCF7NS). A strong co-localization correlation (PCC) between PKC-θ and LSD1 was observed in the nucleus of both aggressive and mesenchymal breast cancer cells.

### Example 12—Role of Phosphorylation of LSD1 in Nuclear Localization

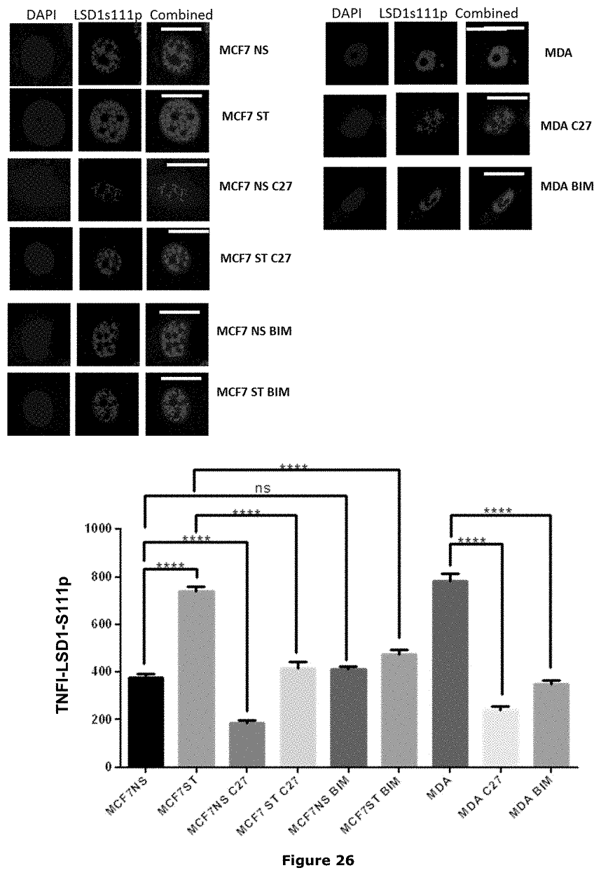

The role of phosphorylation of LSD1 in nuclear localization was assessed using confocal laser scanning microscopy of MDA-MB-231 cells or MCF7 cells treated with vehicle alone or PMA for 60 hours and subsequently treated with the PKC-θ inhibitors C27 (PKC-0 specific inhibitor) or BIM (pan-PKC inhibitor) (FIG. 26). Expression of LSD1 phosphorylated at serine 111 (LSD1s111p) was determined. The expression of LSD1s111p is increased in aggressive (MDA-MB-231 cells) and mesenchymal (MCF7 cells) breast cancer cells. Furthermore, the fluorescent signal of the phosphorylated protein was found to be entirely nuclear. The PKC-θ inhibitors C27 or BIM significantly abrogated LSD1 phosphorylation in both cell lines. Thus, this data indicates that a PKC, especially PKC-0, is involved in phosphorylation of LSD1 at serine 111 and that such phosphorylation is important for nuclear localization.

### Example 13—Expression of Phosphorylated LSD1 in Chemotherapy Resistant Cells

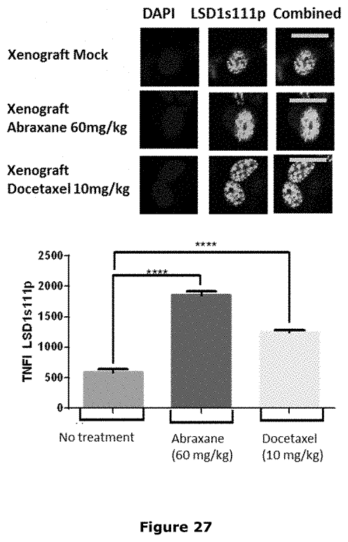

The expression of phosphorylated LSD1 in chemotherapy resistant cells was assessed using confocal laser scanning microscopy of xenografted MDA-MB-231 cells treated with vehicle alone or the chemotherapeutics, Abraxane or Docetaxel (FIG. 27). Surviving chemotherapy resistant MDA-MB-231 cells treated with either Abraxane or Docetaxel displayed a significantly increased nuclear fluorescent signal of phosphorylated LSD1 (LSD1s111p) when compared with cells treated with vehicle alone.

### Example 14—Effect of LSD1 Catalytic Inhibitors on LSD1 Nuclear Localization

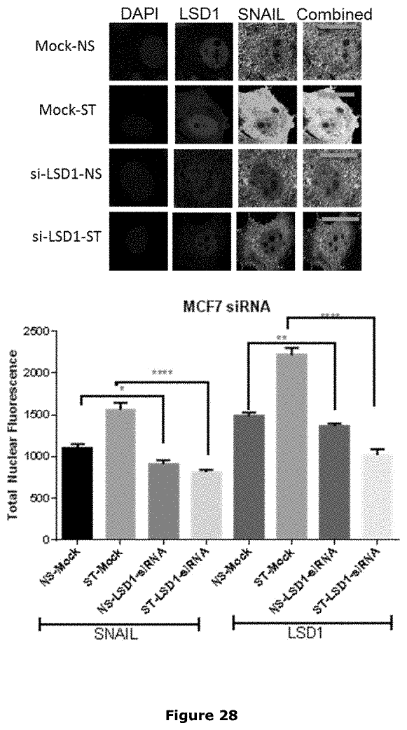

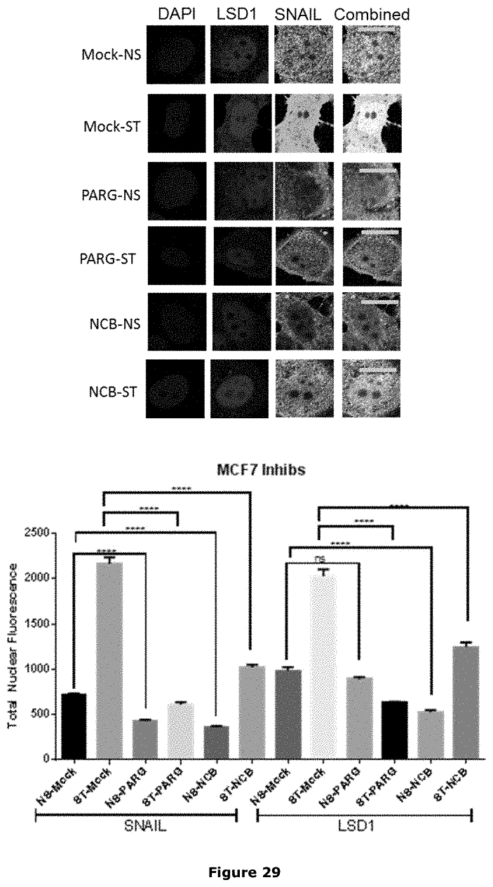

The effect of LSD1 siRNA, or the LSD1 catalytic inhibitors NCD36 (2-[N-(4-phenylbenzenecarbonyl)]amino-6-(trans-2-phenylcyclopropan-1-amino)-N-(3-methylbenzyl)hexanamide hydrochloride; refer to EP 2927212 A1) or Pargyline (Parg) on the nuclear localization of LSD1 and SNAIL was investigated in MCF7 cells treated with vehicle alone or stimulated with PMA for 60 hours, and MDA-MB-231 cells using confocal laser scanning microscopy. In stimulated MCF7 cells, nuclear SNAIL and LSD1 was found to be increased (FIGS. 28 and 29). However, treatment with LSD1 siRNA, NCD36 and pargyline significantly abrogated the nuclear fluorescence signal of SNAIL and LSD1, indicating that the catalytic activity of LSD1 also contributes to LSD1 nuclear localization and is critical for SNAIL expression.

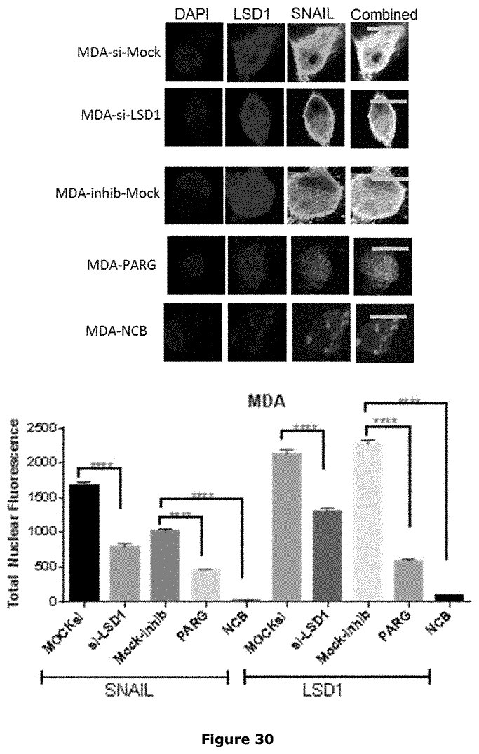

In MDA-MB-231 cells, nuclear SNAIL and LSD1 was found to be increased (FIG. 30). However, treatment with LSD1 siRNA, NCD36 and pargyline significantly abrogated the nuclear fluorescence signal of SNAIL and LSD1. Again, this suggests that the catalytic activity of LSD1 contributes to LSD1 and SNAIL nuclear expression.

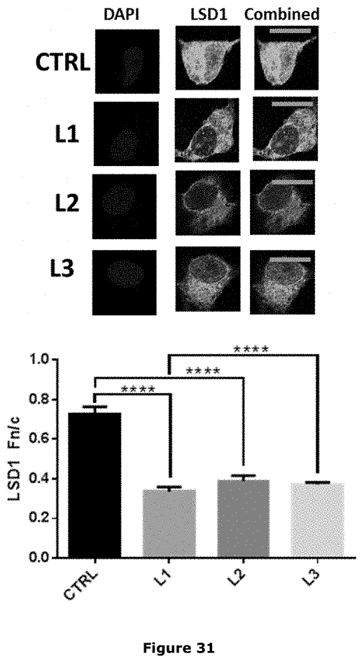

The efficacy of the peptide inhibitors, L1, L2 and L3 (synthesized according to Example 1) was also assessed in MDA-MB-231 cells. Similarly to the LSD1 catalytic inhibitors, L1, L2 and L3 clearly abrogated the nuclear translocation of LSD1 in MDA-MB-231 cells (FIG. 31).

### Materials and Methods

All materials and reagents used are readily available from commercial sources such as Sigma-Aldrich, Santa Cruz Biotechnology, Abcam, etc., unless otherwise indicated.

### Cell Culture

MCF7 and MDA-MB-231 cells were obtained from the American Type Culture Collection (Manassas, Va.). Cells were cultured in DMEM (Invitrogen, Life Technologies, Carlsbad, Calif.) supplemented with 10% FBS, 2 mM L-glutamine, and 1% penicillin-streptomycin-neomycin. MCF7 cells were stimulated with 1.32 ng/ml phorbol 12-myristate 13-acetate (PMA) (Sigma-Aldrich, St Louis, Mo.) or 5 ng/ml recombinant TGF-β1 (R&D Systems, Minneapolis, Minn.) for 60 h. For the co-culture assay MDA-MB-231 cells were stimulated with Jurkat T-cells at defined ratios.

### Immunofluorescence Analysis of Circulating Tumor Cells

Cells were permeabilised by incubating with 2% Triton X-100 for 20 min. Cells were probed with either rabbit antibodies to LSD1s111p (Merck ABE1462), EGFR (AB2430), MET (ab51067), PD-L1 (sc-50298), goat antibodies to PD-L1 (sc-19091), SC-14033), (sc-19096), mouse antibodies to Cytokeratin (Miltenyi 130-090-866), CSV (Abnova H00007431-M08), vimentin (SC-6260) followed by visualisation with secondary anti mouse Alexa-Fluor 568 (A10037), secondary anti rabbit Alexa-Fluor 488 (A21206) or secondary anti goat Alexa-Fluor 633 (A21082). Cover slips were mounted on glass microscope slides with ProLong Diamond Antifade© reagent (Life Technologies). Antibody staining was localised by confocal laser scanning microscopy. Single 0.5 μm sections were obtained using a Nikon x 60 oil immersion lens on the Nikon C1 plus confocal system running NIS-Elements AR 3.2 software. The final image was obtained by averaging four sequential images of the same section. Digital confocal images were analysed using ImageJ software (ImageJ, NIH, Bethesda, Md., USA) to determine the nuclear/cytoplasmic fluorescence ratio (Fn/c) using the equation: Fn/c=(Fn−Fb)/(Fc−Fb), where Fn is nuclear fluorescence, Fc is cytoplasmic fluorescence, and Fb is background fluorescence or the Total Nuclear Fluorescence (TNFI), the Total Cytoplasmic Fluorescence (TCFI) or Total cell fluorescence. ImageJ software with automatic thresholding and manual selection of regions of interest (ROIs) specific for cell nuclei was used to calculate the Pearson's co-efficient correlation (PCC) for each pair of antibodies. PCC values range from: −1=inverse of co-localization, 0=no co-localization, +1=perfect co-localization. Florescence intensity was also measured in a minimum of n=10 cells for each sample set. The Mann-Whitney non-parametric test (GraphPad Prism, GraphPad Software, San Diego, Calif.) was used to determine significant differences between datasets.

### MDA-MB-231 Mice Xenograft Model

Five week old female nude mice were acquired from the Animal Resources Centre (Perth) and were allowed to acclimatize for one week in the animal facility at the John Curtin School of Medical Research (JCSMR) before any experiments were carried out. All experimental procedures were approved by the Australian National University Animal Experimental Ethics Committee (ANU AEEC). MDA-MB-231 human breast carcinoma cells were injected subcutaneously into the right mammary gland (2×106 cells in 25 μL PBS mixed with 25 μL of BD Matrigel Matrix. Tumors were measured using external callipers and calculated using the modified ellipsoidal formula/2 (a/b 2) whereby a=longest diameter and b=shorted diameter. Tumors were allowed to grow to around 50 mm³ before treatments begin (around 15 days). All treatments were given intraperitoneally.

Single Cell Suspension from Tumors and Flow Cytometry Staining

Tumors were excised and collected in ice-cold DMEM supplemented with 2.5% FCS. Tumors were then finely minced using a surgical blade in a petri dish and incubated at 37° C. for 1 hour with shaking in DMEM 2.5% FCS supplemented with collagenase type 4 (Worthington-Biochem, USA) (1 mg of collagenase/1 g of tumor). Digested tumors were spun and resuspended in DMEM 2.5% FCS before being passed through a 0.2 μm filter into a 50 ml tube. Viable cells were then counted using trypan blue. A total number of 2×10⁵ cells were stained for CD44-APC, CD24-PE and Hoescht. Flow cytometry acquisition was done using LSR II. Analysis of flow cytometry staining was done using the FlowJo software.

PBMC and CTC Isolation from Metastatic Breast Cancer Patient Liquid Biopsy

Whole blood was stored in EDTA tubes for circulating tumor cell identification. The RosetteSep™ Human CD45 Depletion Cocktail was used to enrich tumor cells (CTCs) from whole blood by depleting CD45+ cells. Unwanted cells were targeted for depletion with Tetrameric Antibody Complexes recognizing CD45, CD66b and glycophorin A on red blood cells (RBCs). Unwanted cells were then removed via centrifugation over a buoyant density medium Lymphoprep™ (Catalogue #07801). The purified epithelial tumor cells were then extracted as a highly enriched population from the interface between the plasma and the buoyant density medium and were harvested in 20% FBS in PBS. Peripheral blood mononuclear cells (PBMCs) were isolated as well. PBMCs were stimulated with CD28 and P/I for 4 hours followed by co-culture with purified CD45 depleted cells for 12 hours with/without inhibitors of interest.

### Flow Cytometry Analysis of Circulating Tumor Cells (CD45−EpCAM+/CD45−EpCAM+CK+) and MB-MDA-231 Cells (Instrument: BD LSR II Flow Cytometer)

Isolated PBMCs and CTCs were stained with CD45−APC, pan-cytokeratin (CK)-FITC, EpCAM-Percp-Cy5.5 and PD-L1-BV421 antibodies. Co-cultured MDA-MB-231 cells were stained with CD44, CD24 and PD-L1. Flow cytometric analysis was performed on single cell suspensions for using the BD LSR II Flow Cytometer.

### Immunofluorescence Analysis of PD-L1, LSD1, EGFR or MET Expression in MDA-MB-231 Cells or MCF7 Cells in Response to L1, L2 and L3 Treatment

MDA-MB-231 or MCF7 cells were treated with one of L1, L2 or L3 and a control. Cells were then fixed with 3.7% formaldehyde and permeabilized with 2% Triton-X-100, then probed with a primary mouse antibody against LSD1 or a primary rabbit antibody against PD-L1, EGFR or MET, followed by visualization with a secondary goat antibody to mouse or rabbit immunoglobulins conjugated to Alexa-Fluor 488. Confocal laser scanning microscopy was used to measure the Fn/c (ratio of nuclear to cytoplasmic fluorescent intensity) ratio of LSD1 with Fiji-imageJ or the total nuclear fluorescence of PD-L1, EGFR or MET. Values are plotted with significant differences indicated using the Mann-Whitney T-test, with at least 20 cells counted per sample. Representative images for each treatment are shown.

### Flow Cytometry Analysis of CSC Formation in MCF7 and MDA-MB-231 Cells in Response to L1, L2 and L3 Treatment

5×10⁴ MCF7 or MDA-MB-231 cells were seeded with 1 mL of complete DMEM in 12 well plates overnight. MCF7 cells were then treated with LSD1 peptide inhibitors L1, L2 and L3 (50 μM and 100 μM test concentrations) for 24 hours and stimulated by PMA for 60 hours. MDA-MB-231 cells were then treated with L1, L2 and L3 (50 μM and 100 μM test concentrations) for 48 hours. Samples were harvested by trypsinisation followed by washing with DPBS containing 2% HI-FBS. FACS staining were performed using anti-human CD44-APC, anti-human CD24-PE, Hoechst, and anti-human EpCAM antibody cocktails. Data was collected from a BD FACSLSR-II flow cytometer. Treestar FlowJo was used for data analysis.

Immunofluorescence Analysis of PKC-θ and LSD1 Expression in MDA-MB-231 Cells or MCF7 Cells Alone and Treated with PKC Inhibitors

Confocal laser scanning microscopy was performed on either MDA-MB-231 cells treated with vehicle alone or MCF7 cells treated with vehicle alone or PMA for 60 hours. To assess the effect of the PKC inhibitors, C27 (Compound 27; (R)-2-((S)-4-(3-chloro-5-fluoro-6-(1H-pyrazolo[3,4-b]pyridine-3-yl)pyridine-2-yl)piperazin-2-yl)-3-methylbutan-2-ol; see Jimenez et al. (2013) J. Med. Chem., 56(5): 1799-1810) or bisindolylmaleimide (BIM), cells were then treated with C27, BIM or vehicle. Cells were fixed with 3.7% formaldehyde and permeabilized with 2% Triton-X-100, and were then probed with primary rabbit antibody to PKC-θ and a primary mouse antibody to LSD1 or LSD1 phosphorylated at serine 111 (LSD1s111p), respectively, followed by the corresponding secondary antibody conjugated to Alexa-Fluor 488 or Alexa-Fluor 568. Total nuclear fluorescence (TNFI) values for non-stimulated and stimulated MCF7 and MDA-MB-231 cells were calculated. Data shown represent the mean±SE. The plot-profile feature of ImageJ was used to plot the fluorescence signal intensity along a single line spanning the nucleus (n=5 lines per nucleus, 5 individual cells). The average fluorescence signal intensity for the indicated pair of antibodies was plotted for each point on the line with SE. Signal was plotted to compare how the signals for each antibody varied compared to the opposite antibody. The PCC was determined for each plot profile, which indicates the strength of relation between the two fluorochrome signals for at least 20 individual cells ±SE. Colours from representative images correspond to plot profiles.

Immunofluorescence Analysis of LSD1 and SNAIL Expression in MDA-MB-231 Cells or MCF7 Cells in Response to Treatment with LSD1 Inhibitors

Confocal laser scanning microscopy was performed on either MDA-MB-231 cells treated with vehicle alone or MCF7 cells treated with vehicle alone or PMA for 60 h, and subsequently treated with a test inhibitor or vehicle alone. Test inhibitors include LSD1 siRNA or LSD1 catalytic inhibitors NCD36 (2-[N-(4-phenylbenzenecarbonyl)]amino-6-(trans-2-phenylcyclopropan-1-amino)-N-(3-methylbenzyl)hexanamide hydrochloride) or pargyline (Parg). Cells were fixed with 3.7% formaldehyde and permeabilized with 2% Triton-X-100, and then probed with a primary mouse antibody to LSD1 and a primary goat antibody to SNAIL, respectively, followed by the corresponding secondary antibodies conjugated to Alexa-Fluor 488 and Alex-Fluor 563, respectively. TNFI values for MDA-MB-231, non-stimulated MCF7 and stimulated MCF7 cells were calculated. Data shown represent the mean±SE for at least 20 individual cells.

Immunofluorescence Analysis of LSD1 Expression MDA-MB-231 Cells in Response to Treatment with LSD1 Peptide Inhibitors

Confocal laser scanning microscopy was performed on MDA-MB-231 cells treated with a LSD1 peptide inhibitor, L1, L2 or L3, or vehicle alone. Cells were fixed with 3.7% formaldehyde and permeabilized with 2% Triton-X-100, and then probed with a primary mouse antibody to LSD1, followed by the corresponding secondary antibody conjugated to Alexa-Fluor 488. Confocal laser scanning microscopy was used to determine the Fn/c ratio of LSD1 with image-J. Values are plotted with significant differences indicated. Data shown represent the mean±SE for at least 20 individual cells.

Immunofluorescence Analysis of Phosphorylated LSD1 Expression in Xenografted MDA-MB-231 Cells Treated with Chemotherapeutics

The nuclear intensity of LSD1s111p was measured using confocal laser scanning microscopy in xenografted MDA-MB-231 cells treated with vehicle alone or Abraxene (60 mg/kg) or Docetaxel (10 mg/kg). Cells were fixed with 3.7% formaldehyde and permeabilized with 2% Triton-X-100, and then probed with a primary rabbit antibody to LSD1s111p, followed by the corresponding secondary antibody conjugated to Alexa-Fluor 488. Total nuclear fluorescence (TNFI) values for xenografted MDA-MB-231 cells were calculated. Data shown represents the mean±SE for at least 20 individual cells.

The disclosure of every patent, patent application, and publication cited herein is hereby incorporated herein by reference in its entirety.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1-93. (canceled)

94. A method of enhancing an immune response in a subject to a target antigen by an immune-modulating agent, comprising administering an LSD1 inhibitor to the subject.

95. The method according to claim 94, wherein the immune-modulating agent is selected from an antigen that corresponds to at least a portion of the target antigen, an antigen-binding molecule that is immuno-interactive with the target antigen, and an immune-modulating cell that modulates an immune response to the target antigen.

96. The method according to claim 94, wherein the subject has a metastatic cancer or an infection.

97. The method according to claim 94, wherein the LSD1 inhibitor is an isolated or purified proteinaceous molecule comprising, consisting or consisting essentially of a sequence corresponding to residues 108 to 118 of LSD1.

98. The method according to claim 97, wherein the isolated or purified proteinaceous molecule is an isolated or purified proteinaceous molecule represented by Formula I:
Z₁RRTX₁RRKRAKVZ₂ (I)
wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof.

99. A method of treating or preventing a cancer, wherein the cancer comprises at least one PKC and/or LSD1 overexpressing cell, or an infection in a subject, comprising administering to the subject an isolated or purified proteinaceous molecule comprising, consisting or consisting essentially of a sequence corresponding to residues 108 to 118 of LSD1.

100. The method according to claim 99, wherein the PKC and/or LSD1 overexpressing cell is a cancer stem cell tumor cell.

101. The method according to claim 99, wherein the isolated or purified proteinaceous molecule is an isolated or purified proteinaceous molecule represented by Formula I:
Z₁RRTX₁RRKRAKVZ₂ (I)
wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof.

102. A method of inhibiting the nuclear translocation of LSD1 in an LSD1 overexpressing cell, comprising contacting the LSD1 overexpressing cell with an isolated or purified proteinaceous molecule comprising, consisting or consisting essentially of a sequence corresponding to residues 108 to 118 of LSD1.

103. The method according to claim 102, wherein the isolated or purified proteinaceous molecule is an isolated or purified proteinaceous molecule represented by Formula I:
Z₁RRTX₁RRKRAKVZ₂ (I)
wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof.

104. An isolated or purified proteinaceous molecule represented by Formula I:
Z₁RRTX₁RRKRAKVZ₂ (I)
wherein:
“Z₁” and “Z₂” are independently absent or are independently selected from at least one of a proteinaceous moiety comprising from about 1 to about 50 amino acid residues (and all integer residues in between), and a protecting moiety; and
“X₁” is selected from small amino acid residues, including S, T, A, G and modified forms thereof;
wherein the proteinaceous molecule is other than a proteinaceous molecule consisting of the amino acid sequence of SEQ ID NO: 4:
[SEQ ID NO: 4]
EGRRTSRRKRAKVE.

105. The isolated or purified proteinaceous molecule according to claim 104, wherein “X₁” is selected from S and A.

106. The isolated or purified proteinaceous molecule according to claim 104, wherein “Z₁” is a proteinaceous molecule represented by Formula II:
X₂X3X₄ (II)
wherein:
“X₂” is absent or is a protecting moiety;
“X₃” is absent or is selected from any amino acid residue; and
“X4” is selected from any amino acid residue.

107. The isolated or purified proteinaceous molecule according to claim 106, wherein “X₃” is selected from basic amino acid residues including R, K and modified forms thereof.

108. The isolated or purified proteinaceous molecule according to claim 106, wherein “X₄” is selected from aromatic amino acid residues, including F, Y, W and modified forms thereof.

109. The isolated or purified proteinaceous molecule according to claim 104, wherein “Z₂” is absent.

110. The isolated or purified proteinaceous molecule according to claim 104, wherein the isolated or purified proteinaceous molecule of Formula I comprises, consists or consists essentially of an amino acid sequence represented by SEQ ID NO: 1, 2 or 3:
[SEQ ID NO: 1]
RRTSRRKRAKV;
[SEQ ID NO: 2]
RRTARRKRAKV;
or
[SEQ ID NO: 3]
RWRRTARRKRAKV.

111. The isolated or purified proteinaceous molecule according to claim 104, wherein the proteinaceous molecule of Formula I further comprises at least one membrane permeating moiety.

112. The isolated or purified proteinaceous molecule according to claim 111, wherein the membrane permeating moiety is a lipid moiety.

113. The isolated or purified proteinaceous molecule according to claim 112, wherein the membrane permeating moiety is a myristoyl group.

114. The isolated or purified proteinaceous molecule according to claim 111, wherein the membrane permeating moiety is conjugated to the N- or C-terminal amino acid residue of the proteinaceous molecule of Formula I.

115. A composition comprising a proteinaceous molecule according to claim 104 and a pharmaceutically acceptable carrier or diluent.
